SEQUENCE LISTING (184 sequences)

SEQ ID NO: 1 - protein, 199 aa, synthetic construct
  REGION 1..199: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 2 - protein, 199 aa, synthetic construct
  REGION 1..199: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIFDYDPLYKEALQMLRDAGAQVSIMTYDEFKHCWDTFVDHQGCPFQPWDGLDEHSQALSGRLRAILQNQGN

SEQ ID NO: 3 - protein, 199 aa, synthetic construct
  REGION 1..199: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDDDPLYKEALQMLRDAGAQVSIMTYDEFKHCWDTFVDHQGCPFQPWDGLDEHSQALSGRLRAILQNQGN

SEQ ID NO: 4 - protein, 199 aa, synthetic construct
  REGION 1..199: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSAGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 5 - protein, 199 aa, synthetic construct
  REGION 1..199: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYYYDPLYKEALQMLRDAGAQVSIMTYDEFKHCWDTFVDHQGCPFQPWDGLDEHSQALSGRLRAILQNQGN

SEQ ID NO: 6 - protein, 181 aa, synthetic construct
  REGION 1..181: note = Synthetic
MEASPASGPRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 7 - protein, 181 aa, synthetic construct
  REGION 1..181: note = Synthetic
MEASPASGPRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIFDYDPLYKEALQMLRDAGAQVSIMTYDEFKHCWDTFVDHQGCPFQPWDGLDEHSQALSGRLRAILQNQGN

SEQ ID NO: 8 - protein, 181 aa, synthetic construct
  REGION 1..181: note = Synthetic
MEASPASGPRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDDDPLYKEALQMLRDAGAQVSIMTYDEFKHCWDTFVDHQGCPFQPWDGLDEHSQALSGRLRAILQNQGN

SEQ ID NO: 9 - protein, 181 aa, synthetic construct
  REGION 1..181: note = Synthetic
MEASPASGPRHKTYLCYEVERLDNGTSVKMDQHRGFLHNQAKNLLCGFYGRHAELRFLDLVPSLQLDPAQIYRVTWFISWSPCFSAGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 10 - protein, 181 aa, synthetic construct
  REGION 1..181: note = Synthetic
MEASPASGPRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYYYDPLYKEALQMLRDAGAQVSIMTYDEFKHCWDTFVDHQGCPFQPWDGLDEHSQALSGRLRAILQNQGN

SEQ ID NO: 11 - protein, 1399 aa, synthetic construct
  REGION 1..1399: note = Synthetic
MYPYDVPDYASPKKKRKVEASDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKVEAS

SEQ ID NO: 12 - protein, 83 aa, synthetic construct
  REGION 1..83: note = Synthetic
TNLSDIIEKETGKQLVIQESILMLPEEVEEVIGNKPESDILVHTAYDESTDENVMLLTSDAPEYKPWALVIQDSNGENKIKML

SEQ ID NO: 13 - protein, 16 aa, synthetic construct
  REGION 1..16: note = Synthetic
SGSETPGTSESATPES

SEQ ID NO: 14 - protein, 4 aa, synthetic construct
  REGION 1..4: note = Synthetic
SGGS

SEQ ID NO: 15 - protein, 7 aa, synthetic construct
  REGION 1..7: note = Synthetic
PKKKRKV

SEQ ID NO: 16 - protein, 1680 aa, synthetic construct
  REGION 1..1680: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 17 - protein, 1680 aa, synthetic construct
  REGION 1..1680: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIFDYDPLYKEALQMLRDAGAQVSIMTYDEFKHCWDTFVDHQGCPFQPWDGLDEHSQALSGRLRAILQNQGNSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 18 - protein, 1680 aa, synthetic construct
  REGION 1..1680: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDDDPLYKEALQMLRDAGAQVSIMTYDEFKHCWDTFVDHQGCPFQPWDGLDEHSQALSGRLRAILQNQGNSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 19 - protein, 1680 aa, synthetic construct
  REGION 1..1680: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSAGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 20 - protein, 1680 aa, synthetic construct
  REGION 1..1680: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYYYDPLYKEALQMLRDAGAQVSIMTYDEFKHCWDTFVDHQGCPFQPWDGLDEHSQALSGRLRAILQNQGNSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 21 - DNA, 8442 bp, synthetic construct
  misc_feature 1..8442: note = Synthetic
  source 1..8442: mol_type = other DNA
atatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatggaagccagcccagcatccgggcccagacacttgatggatccacacatattcacttccaactttaacaatggcattggaaggcataagacctacctgtgctacgaagtggagcgcctggacaatggcacctcggtcaagatggaccagcacaggggctttctacacaaccaggctaagaatcttctctgtggcttttacggccgccatgcggagctgcgcttcttggacctggttccttctttgcagttggacccggcccagatctacagggtcacttggttcatctcctggagcccctgcttctcctggggctgtgccggggaagtgcgtgcgttccttcaggagaacacacacgtgagactgcgtatcttcgctgcccgcatctatgattacgaccccctatataaggaggcactgcaaatgctgcgggatgctggggcccaagtctccatcatgacctacgatgaatttaagcactgctgggacacctttgtggaccaccagggatgtcccttccagccctgggatggactagatgagcacagccaagccctgagtgggaggctgcgggccattctccagaatcagggaaacagcggcagcgagactcccgggacctcagagtccgccacacccgaaagtgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgactctggtggttctactaatctgtcagatattattgaaaaggagaccggtaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctctggtggttctcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatc

SEQ ID NO: 22 - protein, 199 aa, synthetic construct
  REGION 1..199: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIFEDDPLYKEALQMLRDAGAQVSIMTYDEFKHCWDTFVDHQGCPFQPWDGLDEHSQALSGRLRAILQNQGN

SEQ ID NO: 23 - protein, 199 aa, synthetic construct
  REGION 1..199: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIFYDDPLYKEALQMLRDAGAQVSIMTYDEFKHCWDTFVDHQGCPFQPWDGLDEHSQALSGRLRAILQNQGN

SEQ ID NO: 24 - protein, 199 aa, synthetic construct
  REGION 1..199: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISYSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 25 - protein, 199 aa, synthetic construct
  REGION 1..199: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDYLYKEALQMLRDAGAQVSIMTYDEFKHCWDTFVDHQGCPFQPWDGLDEHSQALSGRLRAILQNQGN

SEQ ID NO: 26 - protein, 199 aa, synthetic construct
  REGION 1..199: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISYSPCFSAGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 27 - protein, 199 aa, synthetic construct
  REGION 1..199: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISYSPCFSWGCAGEVRAFLQENTHVRLRIFAARIYDYDYLYKEALQMLRDAGAQVSIMTYDEFKHCWDTFVDHQGCPFQPWDGLDEHSQALSGRLRAILQNQGN

SEQ ID NO: 28 - protein, 199 aa, synthetic construct
  REGION 1..199: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSAGCAGEVRAFLQENTHVRLRIFAARIYDYDYLYKEALQMLRDAGAQVSIMTYDEFKHCWDTFVDHQGCPFQPWDGLDEHSQALSGRLRAILQNQGN

SEQ ID NO: 29 - protein, 199 aa, synthetic construct
  REGION 1..199: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISYSPCFSAGCAGEVRAFLQENTHVRLRIFAARIFDYDPLYKEALQMLRDAGAQVSIMTYDEFKHCWDTFVDHQGCPFQPWDGLDEHSQALSGRLRAILQNQGN

SEQ ID NO: 30 - protein, 199 aa, synthetic construct
  REGION 1..199: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISYSPCFSAGCAGEVRAFLQENTHVRLRIFAARIYDDDPLYKEALQMLRDAGAQVSIMTYDEFKHCWDTFVDHQGCPFQPWDGLDEHSQALSGRLRAILQNQGN

SEQ ID NO: 31 - protein, 199 aa, synthetic construct
  REGION 1..199: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSAGCAGEVRAFLQENTHVRLRIFAARIFDYDYLYKEALQMLRDAGAQVSIMTYDEFKHCWDTFVDHQGCPFQPWDGLDEHSQALSGRLRAILQNQGN

SEQ ID NO: 32 - protein, 199 aa, synthetic construct
  REGION 1..199: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSAGCAGEVRAFLQENTHVRLRIFAARIYDDDYLYKEALQMLRDAGAQVSIMTYDEFKHCWDTFVDHQGCPFQPWDGLDEHSQALSGRLRAILQNQGN

SEQ ID NO: 33 - protein, 199 aa, synthetic construct
  REGION 1..199: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSAGCAGEVRAFLQENTHVRLRIFAARIFDYDPLYKEALQMLRDAGAQVSIMTYDEFKHCWDTFVDHQGCPFQPWDGLDEHSQALSGRLRAILQNQGN

SEQ ID NO: 34 - protein, 199 aa, synthetic construct
  REGION 1..199: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSAGCAGEVRAFLQENTHVRLRIFAARIYDDDPLYKEALQMLRDAGAQVSIMTYDEFKHCWDTFVDHQGCPFQPWDGLDEHSQALSGRLRAILQNQGN

SEQ ID NO: 35 - protein, 181 aa, synthetic construct
  REGION 1..181: note = Synthetic
MEASPASGPRHKTYLCYEVERLDNGTSVKMDQHRGFLHNQAKNLLCGFYGRHAELRFLDLVPSLQLDPAQIYRVTWFISYSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 36 - protein, 181 aa, synthetic construct
  REGION 1..181: note = Synthetic
MEASPASGPRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDYLYKEALQMLRDAGAQVSIMTYDEFKHCWDTFVDHQGCPFQPWDGLDEHSQALSGRLRAILQNQGN

SEQ ID NO: 37 - protein, 1230 aa, synthetic construct
  REGION 1..1230: note = Synthetic
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVAFLISPVKNSDGIFYDSRNYEAQENAILPKNADANGAYNIARKVLWAIGQFKKAEDEKLDKVKIAISNKEWLEYAQTSVKHGS

SEQ ID NO: 38 - protein, 1307 aa, synthetic construct
  REGION 1..1307: note = Synthetic
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 39 - protein, 1300 aa, synthetic construct
  REGION 1..1300: note = Synthetic
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 40 - protein, 1558 aa, synthetic construct
  REGION 1..1558: note = Synthetic
MPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVAFLISPVKNSDGIFYDSRNYEAQENAILPKNADANGAYNIARKVLWAIGQFKKAEDEKLDKVKIAISNKEWLEYAQTSVKHGSPKKKRKVSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 41 - protein, 1558 aa, synthetic construct
  REGION 1..1558: note = Synthetic
MPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISYSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVAFLISPVKNSDGIFYDSRNYEAQENAILPKNADANGAYNIARKVLWAIGQFKKAEDEKLDKVKIAISNKEWLEYAQTSVKHGSPKKKRKVSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 42 - protein, 1558 aa, synthetic construct
  REGION 1..1558: note = Synthetic
MPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSAGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVAFLISPVKNSDGIFYDSRNYEAQENAILPKNADANGAYNIARKVLWAIGQFKKAEDEKLDKVKIAISNKEWLEYAQTSVKHGSPKKKRKVSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 43 - protein, 1558 aa, synthetic construct
  REGION 1..1558: note = Synthetic
MPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDYLYKEALQMLRDAGAQVSIMTYDEFKHCWDTFVDHQGCPFQPWDGLDEHSQALSGRLRAILQNQGNSGSETPGTSESATPESMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVAFLISPVKNSDGIFYDSRNYEAQENAILPKNADANGAYNIARKVLWAIGQFKKAEDEKLDKVKIAISNKEWLEYAQTSVKHGSPKKKRKVSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 44 - protein, 1558 aa, synthetic construct
  REGION 1..1558: note = Synthetic
MPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISYSPCFSAGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVAFLISPVKNSDGIFYDSRNYEAQENAILPKNADANGAYNIARKVLWAIGQFKKAEDEKLDKVKIAISNKEWLEYAQTSVKHGSPKKKRKVSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 45 - protein, 1558 aa, synthetic construct
  REGION 1..1558: note = Synthetic
MPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISYSPCFSWGCAGEVRAFLQENTHVRLRIFAARIYDYDYLYKEALQMLRDAGAQVSIMTYDEFKHCWDTFVDHQGCPFQPWDGLDEHSQALSGRLRAILQNQGNSGSETPGTSESATPESMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVAFLISPVKNSDGIFYDSRNYEAQENAILPKNADANGAYNIARKVLWAIGQFKKAEDEKLDKVKIAISNKEWLEYAQTSVKHGSPKKKRKVSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 46 - protein, 1558 aa, synthetic construct
  REGION 1..1558: note = Synthetic
MPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSAGCAGEVRAFLQENTHVRLRIFAARIYDYDYLYKEALQMLRDAGAQVSIMTYDEFKHCWDTFVDHQGCPFQPWDGLDEHSQALSGRLRAILQNQGNSGSETPGTSESATPESMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVAFLISPVKNSDGIFYDSRNYEAQENAILPKNADANGAYNIARKVLWAIGQFKKAEDEKLDKVKIAISNKEWLEYAQTSVKHGSPKKKRKVSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 47 - protein, 1558 aa, synthetic construct
  REGION 1..1558: note = Synthetic
MPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISYSPCFSAGCAGEVRAFLQENTHVRLRIFAARIFDYDPLYKEALQMLRDAGAQVSIMTYDEFKHCWDTFVDHQGCPFQPWDGLDEHSQALSGRLRAILQNQGNSGSETPGTSESATPESMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVAFLISPVKNSDGIFYDSRNYEAQENAILPKNADANGAYNIARKVLWAIGQFKKAEDEKLDKVKIAISNKEWLEYAQTSVKHGSPKKKRKVSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 48 - protein, 1558 aa, synthetic construct
  REGION 1..1558: note = Synthetic
MPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISYSPCFSAGCAGEVRAFLQENTHVRLRIFAARIYDDDPLYKEALQMLRDAGAQVSIMTYDEFKHCWDTFVDHQGCPFQPWDGLDEHSQALSGRLRAILQNQGNSGSETPGTSESATPESMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVAFLISPVKNSDGIFYDSRNYEAQENAILPKNADANGAYNIARKVLWAIGQFKKAEDEKLDKVKIAISNKEWLEYAQTSVKHGSPKKKRKVSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 49 - protein, 1558 aa, synthetic construct
  REGION 1..1558: note = Synthetic
MPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSAGCAGEVRAFLQENTHVRLRIFAARIFDYDYLYKEALQMLRDAGAQVSIMTYDEFKHCWDTFVDHQGCPFQPWDGLDEHSQALSGRLRAILQNQGNSGSETPGTSESATPESMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVAFLISPVKNSDGIFYDSRNYEAQENAILPKNADANGAYNIARKVLWAIGQFKKAEDEKLDKVKIAISNKEWLEYAQTSVKHGSPKKKRKVSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 50 - protein, 1558 aa, synthetic construct
  REGION 1..1558: note = Synthetic
MPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSAGCAGEVRAFLQENTHVRLRIFAARIYDDDYLYKEALQMLRDAGAQVSIMTYDEFKHCWDTFVDHQGCPFQPWDGLDEHSQALSGRLRAILQNQGNSGSETPGTSESATPESMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVAFLISPVKNSDGIFYDSRNYEAQENAILPKNADANGAYNIARKVLWAIGQFKKAEDEKLDKVKIAISNKEWLEYAQTSVKHGSPKKKRKVSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 51 - DNA, 23 bp, synthetic construct
  misc_feature 1..23: note = Synthetic
  source 1..23: mol_type = other DNA
cgagggcccggttctccagcagg

SEQ ID NO: 52 - DNA, 23 bp, synthetic construct
  misc_feature 1..23: note = Synthetic
  source 1..23: mol_type = other DNA
cgagggcccggttctccagcagg

SEQ ID NO: 53 - DNA, 23 bp, synthetic construct
  misc_feature 1..23: note = Synthetic
  source 1..23: mol_type = other DNA
cgagggtttggttctccagcagg

SEQ ID NO: 54 - DNA, 23 bp, synthetic construct
  misc_feature 1..23: note = Synthetic
  source 1..23: mol_type = other DNA
ggcactgcggctggaggtggggg

SEQ ID NO: 55 - DNA, 23 bp, synthetic construct
  misc_feature 1..23: note = Synthetic
  source 1..23: mol_type = other DNA
ggcattgcggctggaggtggggg

SEQ ID NO: 56 - DNA, 23 bp, synthetic construct
  misc_feature 1..23: note = Synthetic
  source 1..23: mol_type = other DNA
ggcactgcggctggaggtggggg

SEQ ID NO: 57 - DNA, 23 bp, synthetic construct
  misc_feature 1..23: note = Synthetic
  source 1..23: mol_type = other DNA
ggcccagactgagcacgtgatgg

SEQ ID NO: 58 - DNA, 23 bp, synthetic construct
  misc_feature 1..23: note = Synthetic
  source 1..23: mol_type = other DNA
ggcccagactgagcacgtgatgg

SEQ ID NO: 59 - DNA, 23 bp, synthetic construct
  misc_feature 1..23: note = Synthetic
  source 1..23: mol_type = other DNA
ggcccagactgagcacgtgatgg

SEQ ID NO: 60 - DNA, 23 bp, synthetic construct
  misc_feature 1..23: note = Synthetic
  source 1..23: mol_type = other DNA
ggctcagactgagcacgtgatgg

SEQ ID NO: 61 - DNA, 23 bp, synthetic construct
  misc_feature 1..23: note = Synthetic
  source 1..23: mol_type = other DNA
gagtccgagcagaagaagaaggg

SEQ ID NO: 62 - DNA, 23 bp, synthetic construct
  misc_feature 1..23: note = Synthetic
  source 1..23: mol_type = other DNA
gagtttgagcagaagaagaaggg

SEQ ID NO: 63 - DNA, 23 bp, synthetic construct
  misc_feature 1..23: note = Synthetic
  source 1..23: mol_type = other DNA
gagtttgagcagaagaagaaggg

SEQ ID NO: 64 - DNA, 23 bp, synthetic construct
  misc_feature 1..23: note = Synthetic
  source 1..23: mol_type = other DNA
gagtttgagcagaagaagaaggg

SEQ ID NO: 65 - DNA, 23 bp, synthetic construct
  misc_feature 1..23: note = Synthetic
  source 1..23: mol_type = other DNA
ggaatcccttctgcagcacctgg

SEQ ID NO: 66 - DNA, 23 bp, synthetic construct
  misc_feature 1..23: note = Synthetic
  source 1..23: mol_type = other DNA
ggaatcccttctgcagcacctgg

SEQ ID NO: 67 - DNA, 23 bp, synthetic construct
  misc_feature 1..23: note = Synthetic
  source 1..23: mol_type = other DNA
ggaattccttctgcagcacctgg

SEQ ID NO: 68 - DNA, 23 bp, synthetic construct
  misc_feature 1..23: note = Synthetic
  source 1..23: mol_type = other DNA
ggaatcccttctgcagcacctgg

SEQ ID NO: 69 - DNA, 23 bp, synthetic construct
  misc_feature 1..23: note = Synthetic
  source 1..23: mol_type = other DNA
gaacacaaagcatagactgcggg

SEQ ID NO: 70 - DNA, 23 bp, synthetic construct
  misc_feature 1..23: note = Synthetic
  source 1..23: mol_type = other DNA
gaatataaagcatagactgcggg

SEQ ID NO: 71 - DNA, 23 bp, synthetic construct
  misc_feature 1..23: note = Synthetic
  source 1..23: mol_type = other DNA
gaatataaagcatagactgcggg

SEQ ID NO: 72 - DNA, 23 bp, synthetic construct
  misc_feature 1..23: note = Synthetic
  source 1..23: mol_type = other DNA
gaatataaagcatagactgcggg

SEQ ID NO: 73 - DNA, 23 bp, synthetic construct
  misc_feature 1..23: note = Synthetic
  source 1..23: mol_type = other DNA
ggcccagactgagcacgtgatgg

SEQ ID NO: 74 - DNA, 23 bp, synthetic construct
  misc_feature 1..23: note = Synthetic
  source 1..23: mol_type = other DNA
ggcccagactgagcacgtgatgg

SEQ ID NO: 75 - DNA, 23 bp, synthetic construct
  misc_feature 1..23: note = Synthetic
  source 1..23: mol_type = other DNA
ggcctagactgagcacgtgatgg

SEQ ID NO: 76 - DNA, 23 bp, synthetic construct
  misc_feature 1..23: note = Synthetic
  source 1..23: mol_type = other DNA
ggcctagactgagcacgtgatgg

SEQ ID NO: 77 - DNA, 23 bp, synthetic construct
  misc_feature 1..23: note = Synthetic
  source 1..23: mol_type = other DNA
ggaatcccttctgcagcacctgg

SEQ ID NO: 78 - DNA, 23 bp, synthetic construct
  misc_feature 1..23: note = Synthetic
  source 1..23: mol_type = other DNA
ggaattttttctgcagcacctgg

SEQ ID NO: 79 - DNA, 23 bp, synthetic construct
  misc_feature 1..23: note = Synthetic
  source 1..23: mol_type = other DNA
ggaattccttctgcagcacctgg

SEQ ID NO: 80 - DNA, 23 bp, synthetic construct
  misc_feature 1..23: note = Synthetic
  source 1..23: mol_type = other DNA
ggaattccttctgcagcacctgg

SEQ ID NO: 81 - DNA, 23 bp, synthetic construct
  misc_feature 1..23: note = Synthetic
  source 1..23: mol_type = other DNA
gcgcacggtggcggggtcccagg

SEQ ID NO: 82 - DNA, 23 bp, synthetic construct
  misc_feature 1..23: note = Synthetic
  source 1..23: mol_type = other DNA
aagttcgctaatcccggaactgg

SEQ ID NO: 83 - DNA, 23 bp, synthetic construct
  misc_feature 1..23: note = Synthetic
  source 1..23: mol_type = other DNA
gtaacgagctgcatccccgaggg

SEQ ID NO: 84 - DNA, 23 bp, synthetic construct
  misc_feature 1..23: note = Synthetic
  source 1..23: mol_type = other DNA
cgagggcccggttctccagcagg

SEQ ID NO: 85 - DNA, 23 bp, synthetic construct
  misc_feature 1..23: note = Synthetic
  source 1..23: mol_type = other DNA
tcccgcgggtgggccagggctgg

SEQ ID NO: 86 - DNA, 23 bp, synthetic construct
  misc_feature 1..23: note = Synthetic
  source 1..23: mol_type = other DNA
gctggcagcaagggcggcgctgg

SEQ ID NO: 87 - DNA, 23 bp, synthetic construct
  misc_feature 1..23: note = Synthetic
  source 1..23: mol_type = other DNA
acccacggcggggatcagggtgg

SEQ ID NO: 88 - DNA, 23 bp, synthetic construct
  misc_feature 1..23: note = Synthetic
  source 1..23: mol_type = other DNA
aatgcgggtgcaaggcctgccgg

SEQ ID NO: 89 - DNA, 23 bp, synthetic construct
  misc_feature 1..23: note = Synthetic
  source 1..23: mol_type = other DNA
cgtcgccgatcttcacagggtgg

SEQ ID NO: 90 - DNA, 23 bp, synthetic construct
  misc_feature 1..23: note = Synthetic
  source 1..23: mol_type = other DNA
ctggcgcaacgctgagcagctgg

SEQ ID NO: 91 - DNA, 23 bp, synthetic construct
  misc_feature 1..23: note = Synthetic
  source 1..23: mol_type = other DNA
caggcggacagaagcgcggctgg

SEQ ID NO: 92 - DNA, 23 bp, synthetic construct
  misc_feature 1..23: note = Synthetic
  source 1..23: mol_type = other DNA
aggcagcaaatccagtttgccgg

SEQ ID NO: 93 - DNA, 23 bp, synthetic construct
  misc_feature 1..23: note = Synthetic
  source 1..23: mol_type = other DNA
ggtcagcggactcaccggccagg

SEQ ID NO: 94 - DNA, 23 bp, synthetic construct
  misc_feature 1..23: note = Synthetic
  source 1..23: mol_type = other DNA
tagctcggaggtcgtggcgctgg

SEQ ID NO: 95 - DNA, 23 bp, synthetic construct
  misc_feature 1..23: note = Synthetic
  source 1..23: mol_type = other DNA
ggctagcaccagcgctctgtcgg

SEQ ID NO: 96 - DNA, 23 bp, synthetic construct
  misc_feature 1..23: note = Synthetic
  source 1..23: mol_type = other DNA
gcgcacggtggcggggtcccagg

SEQ ID NO: 97 - DNA, 23 bp, synthetic construct
  misc_feature 1..23: note = Synthetic
  source 1..23: mol_type = other DNA
aagttcgctaatcccggaactgg

SEQ ID NO: 98 - DNA, 23 bp, synthetic construct
  misc_feature 1..23: note = Synthetic
  source 1..23: mol_type = other DNA
gctggcagcaagggcggcgctgg

SEQ ID NO: 99 - DNA, 27 bp, synthetic construct
  misc_feature 1..27: note = Synthetic
  source 1..27: mol_type = other DNA
tttagaagcacatcaaggacattctaa

SEQ ID NO: 100 - DNA, 27 bp, synthetic construct
  misc_feature 1..27: note = Synthetic
  source 1..27: mol_type = other DNA
tttagaagcatattaaggacattctaa

SEQ ID NO: 101 - DNA, 27 bp, synthetic construct
  misc_feature 1..27: note = Synthetic
  source 1..27: mol_type = other DNA
tttagaagcatattaaggacattctaa

SEQ ID NO: 102 - DNA, 27 bp, synthetic construct
  misc_feature 1..27: note = Synthetic
  source 1..27: mol_type = other DNA
tttagaagcatattaaggacattctaa

SEQ ID NO: 103 - DNA, 27 bp, synthetic construct
  misc_feature 1..27: note = Synthetic
  source 1..27: mol_type = other DNA
tttagaagcacatcaaggacattctaa

SEQ ID NO: 104 - DNA, 27 bp, synthetic construct
  misc_feature 1..27: note = Synthetic
  source 1..27: mol_type = other DNA
tttagaagcatattaaggacattctaa

SEQ ID NO: 105 - DNA, 27 bp, synthetic construct
  misc_feature 1..27: note = Synthetic
  source 1..27: mol_type = other DNA
tttagaagcatattaaggacattctaa

SEQ ID NO: 106 - DNA, 27 bp, synthetic construct
  misc_feature 1..27: note = Synthetic
  source 1..27: mol_type = other DNA
tttagaagcacattaaggacattttaa

SEQ ID NO: 107 - DNA, 27 bp, synthetic construct
  misc_feature 1..27: note = Synthetic
  source 1..27: mol_type = other DNA
tttagaagcacatcaaggacattctaa

SEQ ID NO: 108 - DNA, 27 bp, synthetic construct
  misc_feature 1..27: note = Synthetic
  source 1..27: mol_type = other DNA
tttagaagcatattaaggacattctaa

SEQ ID NO: 109 - DNA, 27 bp, synthetic construct
  misc_feature 1..27: note = Synthetic
  source 1..27: mol_type = other DNA
tttagaagcatattaaggacattctaa

SEQ ID NO: 110 - DNA, 27 bp, synthetic construct
  misc_feature 1..27: note = Synthetic
  source 1..27: mol_type = other DNA
tttagaagcatattaaggacattctaa

SEQ ID NO: 111 - DNA, 27 bp, synthetic construct
  misc_feature 1..27: note = Synthetic
  source 1..27: mol_type = other DNA
tttagaagcatattaaggacattctaa

SEQ ID NO: 112 - DNA, 27 bp, synthetic construct
  misc_feature 1..27: note = Synthetic
  source 1..27: mol_type = other DNA
tttagaagcacattaaggacattctaa

SEQ ID NO: 113 - DNA, 27 bp, synthetic construct
  misc_feature 1..27: note = Synthetic
  source 1..27: mol_type = other DNA
tttagaagcatattaaggacattctaa

SEQ ID NO: 114 - DNA, 27 bp, synthetic construct
  misc_feature 1..27: note = Synthetic
  source 1..27: mol_type = other DNA
tttagaagcatattaaggacattctaa

SEQ ID NO: 115 - DNA, 27 bp, synthetic construct
  misc_feature 1..27: note = Synthetic
  source 1..27: mol_type = other DNA
tttagaagcatattaaggacattctaa

SEQ ID NO: 116 - DNA, 27 bp, synthetic construct
  misc_feature 1..27: note = Synthetic
  source 1..27: mol_type = other DNA
tttagaagcacattaaggacattctaa

SEQ ID NO: 117 - DNA, 27 bp, synthetic construct
  misc_feature 1..27: note = Synthetic
  source 1..27: mol_type = other DNA
tttagaagcacattaaggacattctaa

SEQ ID NO: 118 - DNA, 27 bp, synthetic construct
  misc_feature 1..27: note = Synthetic
  source 1..27: mol_type = other DNA
tttagaagcatataaaggacattctaa

SEQ ID NO: 119 - DNA, 27 bp, synthetic construct
  misc_feature 1..27: note = Synthetic
  source 1..27: mol_type = other DNA
tttagaagcacattaaggacattctaa

SEQ ID NO: 120 - DNA, 27 bp, synthetic construct
  misc_feature 1..27: note = Synthetic
  source 1..27: mol_type = other DNA
tttagaagcacattaaggacattctaa

SEQ ID NO: 121 - DNA, 27 bp, synthetic construct
  misc_feature 1..27: note = Synthetic
  source 1..27: mol_type = other DNA
tttagaagcatattaaggacattctaa

SEQ ID NO: 122 - DNA, 27 bp, synthetic construct
  misc_feature 1..27: note = Synthetic
  source 1..27: mol_type = other DNA
tttagaagcacattaaggacattctaa

SEQ ID NO: 123 - DNA, 27 bp, synthetic construct
  misc_feature 1..27: note = Synthetic
  source 1..27: mol_type = other DNA
tttagaagcacattaaggacattctaa

SEQ ID NO: 124 - DNA, 27 bp, synthetic construct
  misc_feature 1..27: note = Synthetic
  source 1..27: mol_type = other DNA
tttagaagcatattaaggacattctaa

SEQ ID NO: 125 - DNA, 27 bp, synthetic construct
  misc_feature 1..27: note = Synthetic
  source 1..27: mol_type = other DNA
tttagaagcacattaaggacattctaa

SEQ ID NO: 126 - DNA, 27 bp, synthetic construct
  misc_feature 1..27: note = Synthetic
  source 1..27: mol_type = other DNA
tttagaagcacattaaggacattctaa

SEQ ID NO: 127 - DNA, 27 bp, synthetic construct
  misc_feature 1..27: note = Synthetic
  source 1..27: mol_type = other DNA
tttcccctcgtcccccttgtgagtacc

SEQ ID NO: 128 - DNA, 27 bp, synthetic construct
  misc_feature 1..27: note = Synthetic
  source 1..27: mol_type = other DNA
tttcccctcgtcccccctgtgagtacc

SEQ ID NO: 129 - DNA, 27 bp, synthetic construct
  misc_feature 1..27: note = Synthetic
  source 1..27: mol_type = other DNA
tttcccctcgtcccccctgtgagtacc

SEQ ID NO: 130 - DNA, 27 bp, synthetic construct
  misc_feature 1..27: note = Synthetic
  source 1..27: mol_type = other DNA
tttcccctcgtcccccctgtgagtacc

SEQ ID NO: 131 - DNA, 27 bp, synthetic construct
  misc_feature 1..27: note = Synthetic
  source 1..27: mol_type = other DNA
tttcccctcgtcccccctgtgagtacc

SEQ ID NO: 132 - DNA, 27 bp, synthetic construct
  misc_feature 1..27: note = Synthetic
  source 1..27: mol_type = other DNA
tttcccctcgtcctttttgtgagtacc

SEQ ID NO: 133 - DNA, 27 bp, synthetic construct
  misc_feature 1..27: note = Synthetic
  source 1..27: mol_type = other DNA
tttcccctcgtcccccttgtgagtacc

SEQ ID NO: 134 - DNA, 27 bp, synthetic construct
  misc_feature 1..27: note = Synthetic
  source 1..27: mol_type = other DNA
tttcccctcgttttttttgtgagtacc

SEQ ID NO: 135 - DNA, 27 bp, synthetic construct
  misc_feature 1..27: note = Synthetic
  source 1..27: mol_type = other DNA
tttcccctcgtcccccctgtgagtacc

SEQ ID NO: 136 - DNA, 27 bp, synthetic construct
  misc_feature 1..27: note = Synthetic
  source 1..27: mol_type = other DNA
tttcccctcgtcccccctgtgagtacc

SEQ ID NO: 137 - DNA, 27 bp, synthetic construct
  misc_feature 1..27: note = Synthetic
  source 1..27: mol_type = other DNA
tttcccctcgttttttttgtgagtacc

SEQ ID NO: 138 - DNA, 27 bp, synthetic construct
  misc_feature 1..27: note = Synthetic
  source 1..27: mol_type = other DNA
tttcccctcgtcctttttgtgagtacc

SEQ ID NO: 139 - DNA, 27 bp, synthetic construct
  misc_feature 1..27: note = Synthetic
  source 1..27: mol_type = other DNA
tttcccctcgtcccccctgtgagtacc

SEQ ID NO: 140 - DNA, 27 bp, synthetic construct
  misc_feature 1..27: note = Synthetic
  source 1..27: mol_type = other DNA
tttcccctcgtcccccctgtgagtacc

SEQ ID NO: 141 - DNA, 27 bp, synthetic construct
  misc_feature 1..27: note = Synthetic
  source 1..27: mol_type = other DNA
tttcccctcgtcccccctgtgagtacc

SEQ ID NO: 142 - DNA, 27 bp, synthetic construct
  misc_feature 1..27: note = Synthetic
  source 1..27: mol_type = other DNA
tttcccctcgtcccccctgtgagtacc

SEQ ID NO: 143 - DNA, 27 bp, synthetic construct
  misc_feature 1..27: note = Synthetic
  source 1..27: mol_type = other DNA
tttcccctcgtcccccctgtgagtacc

SEQ ID NO: 144 - DNA, 27 bp, synthetic construct
  misc_feature 1..27: note = Synthetic
  source 1..27: mol_type = other DNA
tttcccctcgttttttctgtgagtacc

SEQ ID NO: 145 - DNA, 27 bp, synthetic construct
  misc_feature 1..27: note = Synthetic
  source 1..27: mol_type = other DNA
tttcccctcgttccccctgtgagtacc

SEQ ID NO: 146 - DNA, 27 bp, synthetic construct
  misc_feature 1..27: note = Synthetic
  source 1..27: mol_type = other DNA
tttcccctcgttttttttgtgagtacc

SEQ ID NO: 147 - DNA, 27 bp, synthetic construct
  misc_feature 1..27: note = Synthetic
  source 1..27: mol_type = other DNA
tttcccctcgtcccccttgtgagtacc

SEQ ID NO: 148 - DNA, 27 bp, synthetic construct
  misc_feature 1..27: note = Synthetic
  source 1..27: mol_type = other DNA
tttcccctcgtcccccctgtgagtacc

SEQ ID NO: 149 - DNA, 27 bp, synthetic construct
  misc_feature 1..27: note = Synthetic
  source 1..27: mol_type = other DNA
tttcccctcgtcccccctgtgagtacc

SEQ ID NO: 150 - DNA, 27 bp, synthetic construct
  misc_feature 1..27: note = Synthetic
  source 1..27: mol_type = other DNA
tttcccctcgttttttctgtgagtacc

SEQ ID NO: 151 - DNA, 27 bp, synthetic construct
  misc_feature 1..27: note = Synthetic
  source 1..27: mol_type = other DNA
tttcccctcgttttttctgtgagtacc

SEQ ID NO: 152 - DNA, 27 bp, synthetic construct
  misc_feature 1..27: note = Synthetic
  source 1..27: mol_type = other DNA
tttcccctcgttttttctgtgagtacc

SEQ ID NO: 153 - DNA, 27 bp, synthetic construct
  misc_feature 1..27: note = Synthetic
  source 1..27: mol_type = other DNA
tttcccctcgtcccccctgtgagtacc

SEQ ID NO: 154 - DNA, 27 bp, synthetic construct
  misc_feature 1..27: note = Synthetic
  source 1..27: mol_type = other DNA
tttcccctcgtcccccctgtgagtacc

SEQ ID NO: 155 - DNA, 27 bp, synthetic construct
  misc_feature 1..27: note = Synthetic
  source 1..27: mol_type = other DNA
tttcccctcgtcccccctgtgagtacc

SEQ ID NO: 156 - DNA, 27 bp, synthetic construct
  misc_feature 1..27: note = Synthetic
  source 1..27: mol_type = other DNA
tttcccctcgttttttctgtgagtacc

SEQ ID NO: 157 - DNA, 27 bp, synthetic construct
  misc_feature 1..27: note = Synthetic
  source 1..27: mol_type = other DNA
tttcttctcccctctgctggatacctc

SEQ ID NO: 158 - DNA, 27 bp, synthetic construct
  misc_feature 1..27: note = Synthetic
  source 1..27: mol_type = other DNA
tttcttctcctttttgctggatacctc

SEQ ID NO: 159 - DNA, 27 bp, synthetic construct
  misc_feature 1..27: note = Synthetic
  source 1..27: mol_type = other DNA
tttcttctcctttttgctggatacctc

SEQ ID NO: 160 - DNA, 27 bp, synthetic construct
  misc_feature 1..27: note = Synthetic
  source 1..27: mol_type = other DNA
tttcttctcctttttgctggatacctc

SEQ ID NO: 161 - DNA, 27 bp, synthetic construct
  misc_feature 1..27: note = Synthetic
  source 1..27: mol_type = other DNA
tttcttctcccctctgctggatacctc

SEQ ID NO: 162 - DNA, 27 bp, synthetic construct
  misc_feature 1..27: note = Synthetic
  source 1..27: mol_type = other DNA
tttcttctcctttctgctggatacctc

SEQ ID NO: 163 - DNA, 27 bp, synthetic construct
  misc_feature 1..27: note = Synthetic
  source 1..27: mol_type = other DNA
tttcttctcctttttgctggatacctc

SEQ ID NO: 164 - DNA, 27 bp, synthetic construct
  misc_feature 1..27: note = Synthetic
  source 1..27: mol_type = other DNA
tttcttctcctttttgctggatacctc

SEQ ID NO: 165 - DNA, 27 bp, synthetic construct
  misc_feature 1..27: note = Synthetic
  source 1..27: mol_type = other DNA
tttcttctcctttttgctggatacctc

SEQ ID NO: 166 - DNA, 27 bp, synthetic construct
  misc_feature 1..27: note = Synthetic
  source 1..27: mol_type = other DNA
tttcttctcctttttgctggatacctc

SEQ ID NO: 167 - DNA, 27 bp, synthetic construct
  misc_feature 1..27: note = Synthetic
  source 1..27: mol_type = other DNA
tttcttctcctttttgctggatacctc

SEQ ID NO: 168 - DNA, 27 bp, synthetic construct
  misc_feature 1..27: note = Synthetic
  source 1..27: mol_type = other DNA
tttcttctcctttttgctggatacctc

SEQ ID NO: 169 - DNA, 27 bp, synthetic construct
  misc_feature 1..27: note = Synthetic
  source 1..27: mol_type = other DNA
tttcttctcctttttgctggatacctc

SEQ ID NO: 170 - DNA, 27 bp, synthetic construct
  misc_feature 1..27: note = Synthetic
  source 1..27: mol_type = other DNA
tttcttctcctttttgctggatacctc

SEQ ID NO: 171 - DNA, 27 bp, synthetic construct
  misc_feature 1..27: note = Synthetic
  source 1..27: mol_type = other DNA
tttcttctcccttctgctggatacctc

SEQ ID NO: 172 - DNA, 27 bp, synthetic construct
  misc_feature 1..27: note = Synthetic
  source 1..27: mol_type = other DNA
tttcttctcctttttgctggatacctc

SEQ ID NO: 173 - DNA, 27 bp, synthetic construct
  misc_feature 1..27: note = Synthetic
  source 1..27: mol_type = other DNA
tttcttctcttttttgctggatacctc

SEQ ID NO: 174 - DNA, 27 bp, synthetic construct
  misc_feature 1..27: note = Synthetic
  source 1..27: mol_type = other DNA
tttcttctcctttttgctggatacctc

SEQ ID NO: 175 - DNA, 27 bp, synthetic construct
  misc_feature 1..27: note = Synthetic
  source 1..27: mol_type = other DNA
tttcttctcccttttgctggatacctc

SEQ ID NO: 176 - DNA, 27 bp, synthetic construct
  misc_feature 1..27: note = Synthetic
  source 1..27: mol_type = other DNA
tttcttctcttttttgctggatacctc

SEQ ID NO: 177 - DNA, 27 bp, synthetic construct
  misc_feature 1..27: note = Synthetic
  source 1..27: mol_type = other DNA
tttcttctcccttttgctggatacctc

SEQ ID NO: 178 - DNA, 27 bp, synthetic construct
  misc_feature 1..27: note = Synthetic
  source 1..27: mol_type = other DNA
tttcttctcccttttgctggatacctc

SEQ ID NO: 179 - DNA, 27 bp, synthetic construct
  misc_feature 1..27: note = Synthetic
  source 1..27: mol_type = other DNA
tttcttctcctttttgctggatacctc

SEQ ID NO: 180 - DNA, 27 bp, synthetic construct
  misc_feature 1..27: note = Synthetic
  source 1..27: mol_type = other DNA
tttcttctcccttttgctggatacctc

SEQ ID NO: 181 - DNA, 27 bp, synthetic construct
  misc_feature 1..27: note = Synthetic
  source 1..27: mol_type = other DNA
tttcttctcccttttgctggatacctc

SEQ ID NO: 182 - DNA, 27 bp, synthetic construct
  misc_feature 1..27: note = Synthetic
  source 1..27: mol_type = other DNA
tttcttctcttttttgctggatacctc

SEQ ID NO: 183 - DNA, 27 bp, synthetic construct
  misc_feature 1..27: note = Synthetic
  source 1..27: mol_type = other DNA
tttcttctcccttttgctggatacctc

SEQ ID NO: 184 - DNA, 27 bp, synthetic construct
  misc_feature 1..27: note = Synthetic
  source 1..27: mol_type = other DNA
tttcttctcctttttgctggatacctc

(19) United States
(12) Patent Application Publication
(10) Pub. No.: US 2024/0117335 A1
(43) Pub. Date: Apr. 11, 2024

(54) FUSION PROTEINS FOR BASE EDITING

(71) Applicant: ShanghaiTech University, Shanghai (CN)

(72) Inventors: Jia CHEN, Shanghai (CN); Li Yang, Shanghai (CN); Xingxu Huang, Shanghai (CN); Bei Yang, Shanghai (CN); Xiao Wang, Shanghai (CN); Jianan Li, Shanghai (CN)

(21) Appl. No.: 18/525,555

(22) Filed: Nov. 30, 2023

Related U.S. Application Data
(63) Continuation of application No. 16/770,572, filed on Jun. 5, 2020, now Pat. No. 11,884,947.

(30) Foreign Application Priority Data
Feb. 23, 2018 (WO) PCT/CN2018/076991
Aug. 14, 2018 (WO) PCT/CN2018/100411

(51) Int. Cl. C12N 9/78; C12N 9/22; C12N 15/11; C12N 15/90

(52) U.S. Cl. CPC C12N 9/78; C12N 9/22; C12N 15/11; C12N 15/907; C12Y 305/04; C07K 2319/00; C12N 2310/20; C12N 2800/80

(57) ABSTRACT

Provided are fusion proteins that include an apolipoprotein B mRNA editing enzyme catalytic subunit 3A (APOBEC3A) and a clustered regularly interspaced short palindromic repeats (CRISPR)-associated (Cas) protein, optionally further with uracil glycosylase inhibitor (UGI). Such a fusion protein is able to conduct base editing in DNA by deaminating cytosine to uracil, even when the cytosine is in a GpC context or is methylated.

### CROSS REFERENCE TO RELATED APPLICATIONS

This application is a continuation of U.S. application Ser. No. 16/770,572, filed Jun. 5, 2020, which is a U.S. National Stage Application under 37 U.S.C. 371 of International Application No. PCT/CN2019/075897, filed Feb. 22, 2019, which claim priority to PCT/CN2018/100411, filed Aug. 14, 2018 and PCT/CN2018/076991, filed Feb. 23, 2018, the content of each of which is hereby incorporated by reference in its entirety.

### REFERENCE TO AN ELECTRONIC SEQUENCE LISTING

The contents of the electronic sequence listing (49BD-268973-US2 Sequence Listing.xml; Size: 275,675 bytes; and Date of Creation: Nov. 29, 2023) is herein incorporated by reference in its entirety.

### BACKGROUND

Genome editing is a type of genetic engineering in which DNA is inserted, deleted or replaced in the genome of a living organism using engineered nucleases (molecular scissors). Utilizing genome editing tools to genetically manipulate the genome of cells and living organism has broad application interest in life sciences research, biotechnology/agricultural technology development and most importantly pharmaceutical/clinical innovation. For example, genome editing can be used to correct driver mutations underlying genetic diseases and thereby resulting in complete cure of these diseases in a living organism; genome editing can also be applied to engineer the genome of crops, thus increasing the yield of crops and conferring crops resistance to environmental contamination or pathogen infection; likewise, microbial genome transformation through accurate genome editing is of great significance in the development of renewable bio-energy.

CRISPR/Cas (Clustered regularly interspaced short palindromic repeats/CRISPR-associated protein) system has been the most powerful genomic editing tool since its conception for its unparalleled editing efficiency, convenience and the potential applications in living organism. Directed by guide RNA (gRNA), a Cas nuclease can generate DNA double strand breaks (DSBs) at the targeted genomic sites in various cells (both cell lines and cells from living organisms). These DSBs are then repaired by the endogenous DNA repair system, which could be utilized to perform desired genome editing.

In general, two major DNA repair pathways could be activated by DSBs, non-homologous end joining (NHEJ) and homology-directed repair (HDR). NHEJ can introduce random insertions/deletions (indels) in the genomic DNA region around the DSBs, thereby leading to open reading frame (ORF) shift and ultimately gene inactivation. In contrast, when HDR is triggered, the genomic DNA sequence at target site could be replaced by the sequence of the exogenous donor DNA template through a homologous recombination mechanism, which can result in the correction of genetic mutation.

However, the practical efficiency of HDR-mediated gene correction is low (normally <5%) because the occurrence of homologous recombination is both cell type-specific and cell cycle-dependent and NHEJ is triggered more frequently than HDR is. The relatively low efficiency of HDR therefore limited the translation of CRISPR/Cas genome editing tools in the field of precision gene therapy (diseases-driven gene correction).

Base editors (BE), which integrate the CRISPR/Cas system with the APOBEC (apolipoprotein B mRNA editing enzyme, catalytic polypeptide-like) cytosine deaminase family, were recently invented that greatly enhanced the efficiency of CRISPR/Cas9-meditated gene correction. Through fusion with Cas9 nickase (nCas9), the cytosine (C) deamination activity of rat APOBEC1 (rA1) can be purposely directed to the target bases in genome and to catalyze C to Thymine (T) substitutions at these bases.

However, current rA1-based BEs cannot efficiently edit C that follows a G (i.e., C of GpC), thereby limiting the genome targeting breadth. Therefore, creating new BEs that can efficiently edit C of GpC is highly desirable. Such new BEs will enable us to perform efficient base editing in a broader genomic space of various living organisms. Importantly, the high efficiency of such BEs on C of GpC will promote clinical translation, particularly in gene therapies that involve restoring disease-related GpT-to-GpC mutations.

### SUMMARY

The present disclosure demonstrates that when an apolipoprotein B mRNA editing enzyme catalytic subunit 3A (APOBEC3A or A3A) is fused to a clustered regularly interspaced short palindromic repeats (CRISPR)-associated (Cas) protein, optionally further with uracil glycosylase inhibitor (UGI), the resulting fusion protein is able to efficiently deaminate cytosine's to uracil's resulting in C to T substitution. Such base editing, surprisingly and unexpectedly, was effective even when the C follows a G (i.e., in a GpC dinucleotide context) or when the C is methylated. The editing efficiency can be further increased when the A3A includes a few tested mutations. This has significant clinical significance as cytosine methylation is common in living cells.

In conventional base editors, Cas9 is the commonly used DNA endonuclease. The Cas12a (Cpf1) has the advantage of recognizing A/T rich sequence when used together with APOBEC1 in base editors. In another surprising discovery, when APOBEC1 was replaced with A3A, the editing efficiency was greatly increased. Yet, the editing efficiency of such a Cas12a-A3A can be further increased when the A3A includes a few tested mutations.

Accordingly, in one embodiment, the present disclosure provides a fusion protein comprising a first fragment comprising an apolipoprotein B mRNA editing enzyme catalytic subunit 3A (APOBEC3A) and a second fragment comprising a clustered regularly interspaced short palindromic repeats (CRISPR)-associated (Cas) protein. In some embodiments, the fusion protein further comprises a uracil glycosylase inhibitor (UGI).

Preferably, the fusion protein has fewer than 3000, 2500, 2200, 2100, 2000, 1900, 1800, 1700, 1600, or 1500 amino acid residues in total.

In some embodiments, the APOBEC3A is a wildtype human APOBEC3A or a mutant of human APOBEC3A having a mutation selected from the group consisting of Y130F, D131Y, D131E, Y132D, W104A, W98Y, P134Y and combinations thereof, according to residue numbering in SEQ ID NO:1, wherein the mutant retains cytidine deaminase activity.

In some embodiments, the APOBEC3A is a mutant human APOBEC3A having mutations selected from the group consisting of Y130F+D131E+Y132D, Y130F+D131Y+Y132D, W98Y+W104A, W98Y+P134Y, W104A+P134Y, W104A+Y130F, W104A+Y132D, W98Y+W104A+Y130F, W98Y+W104A+Y132D, W104A+Y130F+P134Y, and W104A+Y132D+P134Y, according to residue numbering in SEQ ID NO:1.

In some embodiments, the APOBEC3A comprises the amino acid sequence of SEQ ID NO:1 or has at least 90% sequence identity to amino acid residues 29-199 of SEQ ID NO:1 and retains cytidine deaminase activity. In some embodiments, the APOBEC3A comprises an amino acid sequence selected from the group consisting of SEQ ID NO:1-10 and 22-36.

In some embodiments, the Cas protein is selected from the group consisting of SpCas9, FnCas9, St1Cas9, St3Cas9, NmCas9, SaCas9, AsCpf1, LbCpf1, FnCpf1, VQR SpCas9, EQR SpCas9, VRER SpCas9, RHA FnCas9, KKH SaCas9, NmeCas9, StCas9, CjCas9, AsCpf1, FnCpf1, SsCpf1, PcCpf1, BpCpf1, CmtCpf1, LiCpf1, PmCpf1, Pb3310Cpf1, Pb4417Cpf1, BsCpf1, EeCpf1, BhCas12b, AkCas12b, EbCas12b, LsCas12b, RfCas13d, LwaCas13a, PspCas13b, PguCas13b, RanCas13b, CasX, and CasY. In some embodiments, the Cas protein is a mutant of protein selected from the group consisting of SpCas9, FnCas9, St1Cas9, St3Cas9, NmCas9, SaCas9, AsCpf1, LbCpf1, FnCpf1, VQR SpCas9, EQR SpCas9, VRER SpCas9, RHA FnCas9, KKH SaCas9, NmeCas9, StCas9, CjCas9, AsCpf1, FnCpf1, SsCpf1, PcCpf1, BpCpf1, CmtCpf1, LiCpf1, PmCpf1, Pb3310Cpf1, Pb4417Cpf1, BsCpf1, EeCpf1, BhCas12b, AkCas12b, EbCas12b, LsCas12b, RfCas13d, LwaCas13a, PspCas13b, PguCas13b, RanCas13b, CasX, and CasY, wherein the mutant retains the DNA-binding capability but does not introduce double strand DNA breaks. In some embodiments, the mutant is capable of introducing a nick to one of the strands of a double stranded DNA bound by the mutant. In some embodiments, the Cas protein comprises the amino acid sequence of any one of SEQ ID NO:11 and 37-39.

In some embodiments, the UGI comprises the amino acid sequence of SEQ ID NO:12 or has at least at least 90% sequence identity to SEQ ID NO:12 and retains the uracil glycosylase inhibition activity.

In some embodiments, the first fragment is at the N-terminal side of the second fragment. In some embodiments, the first fragment is at the N-terminal side of the second fragment which is at the N-terminal side of the UGI.

In some embodiments, the fusion protein further comprises a peptide linker between the first fragment and the second fragment. In some embodiments, the peptide linker has from 1 to 100 amino acid residues. In some embodiments, at least 10%, 20%, 30%, 40%, 50%, 60%, 70%, 80% or 90% of the amino acid residues of peptide linker are amino acid residues selected from the group consisting of alanine, glycine, cysteine, and serine. In some embodiments, the peptide linker has an amino acid sequence of SEQ ID NO:13 or 14. In some embodiments, the fusion protein further comprises a nuclear localization sequence.

Non-limiting examples of fusion proteins include those having an amino acid sequence selected from the group consisting of SEQ ID NO:16-20 and 40-50.

In another embodiment, a fusion protein is provided that comprises a first fragment comprising an apolipoprotein B mRNA editing enzyme catalytic subunit 3A (APOBEC3A) and a second fragment comprising a CRISPR-associated endonuclease in Prevotella and Francisella 1 (Cpf1). In some embodiments, the Cpf1 is catalytically inactive.

The Cpf1 (Cas12a) can be selected from the group consisting of AsCpf1, LbCpf1, and FnCpf1, in some embodiments. In a specific embodiment, the Cpf1 is a catalytically inactive Lachnospiraceae bacterium Cpf1 (dLbCpf1).

In some embodiments, the APOBEC3A is a wildtype human APOBEC3A or a mutant of human APOBEC3A having a mutation selected from the group consisting of Y130F, D131Y, D131E, Y132D, W104A, W98Y, P134Y and combinations thereof, according to residue numbering in SEQ ID NO:1, wherein the mutant retains cytidine deaminase activity.

Also provided is a polynucleotide that encodes a fusion protein of the present disclosure. Still, in another embodiment, provided is a composition comprising a fusion protein of the present disclosure and a pharmaceutically acceptable carrier. In some embodiments, the composition further comprises a guide RNA.

Methods of using the fusion proteins and compositions are also provided. In one embodiment, a method for editing a target polynucleotide is provided, comprising contacting to the target polynucleotide a fusion protein of the present disclosure and a guide RNA having at least partial sequence complementarity to the target polynucleotide, wherein the editing comprises deamination of a cytosine (C) in the target polynucleotide. In some embodiments, the C is in a GpC context. In some embodiments, the C is methylated. In some embodiments, the contacting is in vitro, ex vivo, or in vivo. In some embodiments, the method further comprises contacting to the target polynucleotide with a uracil glycosylase inhibitor (UGI) not fused to a Cas protein.

### BRIEF DESCRIPTION OF THE DRAWINGS

FIG. 1A-B. Construction and performance of hA3A-BE. Panel A: Schematic diagram illustrating the co-expression of BE3/sgRNA or hA3A-BE/sgRNA. Panel B: Comparing to the co-expression of BE3/sgRNA, the co-expression of hA3A-BE/sgRNA achieved more efficient base editing on the C of GpC in the sgRNA targeted genomic regions (sgFANCF-M-L6 and sgSITE4). Dashed boxes represent the cytosine's locating in the context of GpC. Sequences as shown in panel B, from left column to right column and from top to down, are SEQ ID NO:51-56.

FIG. 2A-B. Construction and performance of hA3A-BE-Y130F and hA3A-BE-Y132D. Panel A: Schematic diagram illustrating the co-expression of hA3A-BE/sgRNA, hA3A-BE-Y130F/sgRNA or hA3A-BE-Y132D/sgRNA. Panel B: Comparing to the co-expression of hA3A-BE/sgRNA, the co-expression of hA3A-BE-Y130F/sgRNA or hA3A-BE-Y132D/sgRNA induced base editing in more narrowed windows in the sgRNA targeted genomic regions (sgSITE3 and sgEMX1). Dashed boxes represent the base editing windows. Sequences as shown in panel B, from left column to right column and from top to down, are SEQ ID NO:57-64.

FIG. 3A-B. Construction and performance of hA3A-BE-W104A and hA3A-BE-D131Y. Panel A: Schematic diagram illustrating the co-expression of hA3A-BE/sgRNA, hA3A-BE-W104A/sgRNA or hA3A-BE-D131Y/sgRNA. Panel B: Comparing to the co-expression of hA3A-BE/sgRNA, the co-expression of hA3A-BE-W104A/sgRNA or hA3A-BE-D131Y/sgRNA induced more efficient base editing in the sgRNA targeted genomic regions (sgFANCF and sgSITE2). Dashed boxes represent the edited cytosine's. Sequences as shown in panel B, from left column to right column and from top to down, are SEQ ID NO:65-72.

FIG. 4A-B. Construction and performance of hA3A-BE-Y130E-D131E-Y132D and hA3A-BE-Y130E-D131Y-Y132D. Panel A: Schematic diagram illustrating the co-expression of hA3A-BE/sgRNA, hA3A-BE-Y130E-D131E-Y132D/sgRNA or hA3A-BE-Y130E-D131Y-Y132D/sgRNA. Panel B: Comparing to the co-expression of hA3A-BE/sgRNA, the co-expression of hA3A-BE-Y130E-D131E-Y132D/sgRNA or hA3A-BE-Y130E-D131Y-Y132D/sgRNA induced base editing in more narrowed windows in the sgRNA targeted genomic regions (sgFANCF and sgSITE3). Dashed boxes represent the edited cytosine's. Sequences as shown in panel B, from left column to right column and from top to down, are SEQ ID NO:73-80.

FIG. 5a-h. hA3A-BE3 induces efficient base editing in methylated region and in GpC context. (a) Distribution of BE-editable T-to-C (or A-to-G) variants. Potentially editable cytosines (underlined) are sub-classified according to their 3′ adjacent bases. (b) Screening of BEs for efficient base editing in a high-methylation background. A series of new BEs were constructed by fusing different APOBEC/AID deaminases with Cas9 nickase (nCas9) and uracil DNA glycosylase inhibitor (UGI). (c) Cumulative base editing frequencies induced by different BEs in unmethylated and methylated vectors. A commonly used rA1-based BE3 was chosen for comparison. Means±s.d. were from three (six for hA3A-BE3) independent experiments. (d) Immunoblots of BE3 and hA3A-BE3 co-transfected with unmethylated or methylated vectors. Tubulin was used as a loading control and immunoblot images are representative of three independent experiments. (e) Comparison of base editing efficiencies induced by BE3 and hA3A-BE3 in genomic regions with natively high levels of DNA methylation. C-to-T editing frequencies of indicated cytosines were determined individually. Target site sequences are shown with the BE3 editing window (position 4-8, setting the base distal to the PAM as position 1) in pink, PAM in cyan and CpG site in capital. Shaded gray, guanines at 5′ end of editable cytosines. NT, native HEK293T cells with no treatment. (f) Statistical analysis of normalized C-to-T editing frequencies in regions with natively high levels of DNA methylation shown in (e), setting the ones induced by BE3 as 100%. n=48 samples from three independent experiments. (g) Comparison of base editing efficiencies induced by BE3 and hA3A-BE3 at C of GpC in genomic regions with natively low levels of DNA methylation. (h) Statistical analysis of normalized C-to-T editing frequencies at GpC sites in regions with natively low levels of DNA methylation shown in (g), setting the ones induced by BE3 as 100%. n=24 samples from three independent experiments. (e,g) Means±s.d. were from three independent experiments. (f,h) P value, one-tailed Student's t test. The median and interquartile range (IQR) are shown. Sequences as shown in FIG. 5e are SEQ ID NO:81-89. Sequences as shown in FIG. 5g are SEQ ID NO:90-95.

FIG. 6a-i. Improvements in hA3A-BE3. (a) Comparison of base editing efficiencies induced by BE3, hA3A-BE3, hA3A-BE3-Y130F and hA3A-BE3-Y132D in genomic regions with natively high levels of DNA methylation. Target site sequences are shown with the overlapped editing window (position 4-7) in pink, PAM in cyan and CpG site in capital. NT, native HEK293T cells with no treatment. (b) Statistical analysis of normalized C-to-T editing frequencies in the overlapped editing window shown in (a), setting the ones induced by BE3 as 100%. n=12 samples from three independent experiments. (c) Comparison of base editing efficiencies induced by BE3, hA3A-BE3, hA3A-BE3-Y130F and hA3A-BE3-Y132D at C of GpC in the overlapped editing window in genomic regions with natively low levels of DNA methylation. (d) Statistical analysis of normalized C-to-T editing frequencies shown in (c), setting the ones induced by BE3 as 100%. n=9 samples from three independent experiments. (e) Immunoblots of BEs transfected into HEK293T cells. Tubulin was used as a loading control and immunoblot images are representative of three independent experiments. (f) Comparison of base editing efficiencies induced by hA3A-BE3-Y130F, hA3A-eBE-Y130F, hA3A-BE3-Y132D and hA3A-eBE-Y132D at C of GpC in the overlapped editing window in genomic regions with natively low levels of DNA methylation. (g) Statistical analysis of normalized C-to-T editing frequencies shown in (f), setting the ones induced by hA3A-BE3-Y130F (left) or hA3A-BE3-Y132D (right) as 100%. n=9 samples from three independent experiments. (h,i) Comparison of product purity (h) and indels (i) yielded by hA3A-BE3-Y130F, hA3A-eBE-Y130F, hA3A-BE3-Y132D and hA3A-eBE-Y132D in genomic DNA regions with natively low levels of DNA methylation. Asterisk denotes an unusually high basal indel frequency (or amplification, sequencing or alignment artifact) at the examined VEGFA-M-c site in NT. (a,c,f,i) Means±s.d. were from three independent experiments. (b,d,g) P value, one-tailed Student's t test. The median and IQR are shown. Sequences as shown in FIG. 6a are SEQ ID NO:96-98.

FIGS. 7A-B and 8A-B show the vector structures of each of the tested base editors and charting showing their editing efficiencies on the target DYRK1A gene.

FIGS. 9A-B and 10A-B show the vector structures of each of the tested base editors and charting showing their editing efficiencies on the target SITE6 gene.

FIGS. 11A-B and 12A-B show the vector structures of each of the tested base editors and charting showing their editing efficiencies on the target RUNX1 gene.

FIG. 13-18 show the sequencing results for Examples 3-5. Sequences as shown in FIG. 13, from left column to right column and from top to down, are SEQ ID NO:99-114. Sequences as shown in FIG. 14, from left column to right column and from top to down, are SEQ ID NO:115-126. Sequences as shown in FIG. 15, from left column to right column and from top to down, are SEQ ID NO:127-142. Sequences as shown in FIG. 16, from left column to right column and from top to down, are SEQ ID NO:143-156. Sequences as shown in FIG. 17, from left column to right column and from top to down, are SEQ ID NO:157-172. Sequences as shown in FIG. 18, from left column to right column and from top to down, are SEQ ID NO:173-184.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

The term “isolated” as used herein with respect to cells, nucleic acids, such as DNA or RNA, refers to molecules separated from other DNAs or RNAs, respectively, that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to cells or polypeptides which are isolated from other cellular proteins or tissues. Isolated polypeptides is meant to encompass both purified and recombinant polypeptides.

As used herein, the term “recombinant” as it pertains to polypeptides or polynucleotides intends a form of the polypeptide or polynucleotide that does not exist naturally, a non-limiting example of which can be created by combining polynucleotides or polypeptides that would not normally occur together.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Biologically equivalent polynucleotides are those having the above-noted specified percent homology and encoding a polypeptide having the same or similar biological activity.

The term “an equivalent nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference polypeptide or polynucleotide. In some aspects, the equivalent sequence retains the activity (e.g., epitope-binding) or structure (e.g., salt-bridge) of the reference sequence.

Hybridization reactions can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A non-limiting example of a physiological condition is the temperature, ionic strength, pH and concentration of Mg′ normally found in a cell.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching. The term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

### Fusion Proteins

The current rA1-based BEs (base editors) cannot efficiently edit C in methylated regions or in the context of GpC, which limits the use of base editing. The present disclosure provides fusion molecules that combine an apolipoprotein B mRNA editing enzyme catalytic subunit 3A (APOBEC3A or A3A) and a clustered regularly interspaced short palindromic repeats (CRISPR)-associated (Cas) protein, optionally further with uracil glycosylase inhibitor (UGI).

The resulting fusion protein is able to efficiently deaminate cytosine's to uracil's resulting in C to T substitution. Such base editing, surprisingly and unexpectedly, was effective even when the C follows a G (i.e., in a GpC dinucleotide context) and/or even when it is in a methylated region. This has significant clinical significance as cytosine methylation is common in living cells.

In accordance with one embodiment of the present disclosure, provided is a fusion protein comprising a first fragment comprising an apolipoprotein B mRNA editing enzyme catalytic subunit 3A (APOBEC3A) and a second fragment comprising a clustered regularly interspaced short palindromic repeats (CRISPR)-associated (Cas) protein.

APOBEC3A, also referred to as apolipoprotein B mRNA editing enzyme catalytic subunit 3A or A3A, is a protein of the APOBEC3 family found in humans, non-human primates, and some other mammals. The APOBEC3A protein lacks the zinc binding activity of other family members. In human, isoform a (NP 663745.1; SEQ ID NO:1) and isoform b (NP 001257335.1; SEQ ID NO:6) both are active, while isoform a includes a few more residues close to the N-terminus. The term “APOBEC3A” also encompasses variants and mutants that have certain level (e.g., 70%, 75%, 80%, 85%, 90%, 95%, 98%, 99%) of sequence identity to a wildtype mammalian APOBEC3A and retains its cytidine deaminating activity.

As demonstrated in the experimental examples, certain mutants (e.g., Y130F (SEQ ID NO:2), Y132D (SEQ ID NO:3), W104A (SEQ ID NO:4), D131Y (SEQ ID NO:5), D131E (SEQ ID NO:22), W98Y (SEQ ID NO:24), W104A (SEQ ID NO:25), and P134Y (SEQ ID NO:26)) even outperformed the wildtype human APOBEC3A. Furthermore, a number of tested combinations of these mutations also exhibited great performances. Moreover, although not specifically tested, the same mutations are believed to also work in the isoform b of A3A. Examples of such variants and mutants are provided in Table 1 below.

TABLE 1
Examples of APOBEC3A Sequences

| Name | Sequence | SEQ ID NO: |
|---|---|---|
| Human | 1 MEASPASGPR HLMDPHIFTS NFNNGIGRHK TYLCYEVERL DNGTSVKMDQ | 1 |
| APOBEC3A | 51 HRGFLHNQAK NLLCGFYGRH AELRFLDLVP SLQLDPAQIY RVTWFISWSP | |
| isoform a | 101 CFSWGCAGEV RAFLQENTHV RLRIFAARIY DYDPLYKEAL QMLRDAGAQV | |
| wildtype | 151 SIMTYDEFKH CWDTFVDHQG CPFQPWDGLD EHSQALSGRL RAILQNQGN | |
| Human | 1 MEASPASGPR HLMDPHIFTS NFNNGIGRHK TYLCYEVERL DNGTSVKMDQ | 2 |
| APOBEC3A | 51 HRGFLHNQAK NLLCGFYGRH AELRFLDLVP SLQLDPAQIY RVTWFISWSP | |
| isoform a | 101 CFSWGCAGEV RAFLQENTHV RLRIFAARIF DYDPLYKEAL QMLRDAGAQV | |
| Y130F | 151 SIMTYDEFKH CWDTFVDHQG CPFQPWDGLD EHSQALSGRL RAILQNQGN | |
| Human | 1 MEASPASGPR HLMDPHIFTS NFNNGIGRHK TYLCYEVERL DNGTSVKMDQ | 3 |
| APOBEC3A | 51 HRGFLHNQAK NLLCGFYGRH AELRFLDLVP SLQLDPAQIY RVTWFISWSP | |
| isoform a | 101 CFSWGCAGEV RAFLQENTHV RLRIFAARIY DDDPLYKEAL QMLRDAGAQV | |
| Y132D | 151 SIMTYDEFKH CWDTFVDHQG CPFQPWDGLD EHSQALSGRL RAILQNQGN | |
| Human | 1 MEASPASGPR HLMDPHIFTS NFNNGIGRHK TYLCYEVERL DNGTSVKMDQ | 4 |
| APOBEC3A | 51 HRGFLHNQAK NLLCGFYGRH AELRFLDLVP SLQLDPAQIY RVTWFISWSP | |
| isoform a | 101 CFSAGCAGEV RAFLQENTHV RLRIFAARIY DYDPLYKEAL QMLRDAGAQV | |
| W104A | 151 SIMTYDEFKH CWDTFVDHQG CPFQPWDGLD EHSQALSGRL RAILQNQGN | |
| Human | 1 MEASPASGPR HLMDPHIFTS NFNNGIGRHK TYLCYEVERL DNGTSVKMDQ | 5 |
| APOBEC3A | 51 HRGFLHNQAK NLLCGFYGRH AELRFLDLVP SLQLDPAQIY RVTWFISWSP | |
| isoform a | 101 CFSWGCAGEV RAFLQENTHV RLRIFAARIY YYDPLYKEAL QMLRDAGAQV | |
| D131Y | 151 SIMTYDEFKH CWDTFVDHQG CPFQPWDGLD EHSQALSGRL RAILQNQGN | |
| Human | 1 MEASPASGPR HKTYLCYEVE RLDNGTSVKM DQHRGFLHNQ AKNLLCGFYG | 6 |
| APOBEC3A | 51 RHAELRFLDL VPSLQLDPAQ IYRVTWFISW SPCFSWGCAG EVRAFLQENT | |
| isoform b | 101 HVRLRIFAAR IYDYDPLYKE ALQMLRDAGA QVSIMTYDEF KHCWDTFVDH | |
| wildtype | 151 QGCPFQPWDG LDEHSQALSG RLRAILQNQG N | |
| Human | 1 MEASPASGPR HKTYLCYEVE RLDNGTSVKM DQHRGFLHNQ AKNLLCGFYG | 7 |
| APOBEC3A | 51 RHAELRFLDL VPSLQLDPAQ IYRVTWFISW SPCFSWGCAG EVRAFLQENT | |
| isoform b | 101 HVRLrfeRIFAAR IFDYDPLYKE ALQMLRDAGA QVSIMTYDEF KHCWDTFVDH | |
| Y112F | 151 QGCPFQPWDG LDEHSQALSG RLRAILQNQG N | |
| Human | 1 MEASPASGPR HKTYLCYEVE RLDNGTSVKM DQHRGFLHNQ AKNLLCGFYG | 8 |
| APOBEC3A | 51 RHAELRFLDL VPSLQLDPAQ IYRVTWFISW SPCFSWGACG EVRAFLQENT | |
| isoform b | 101 HVRLRIFAAR IYDDDPLYKE ALQMLRDAGA QVSIMTYDEF KHCWDTFVDH | |
| Y114D | 151 QGCPFQPWDG LDEHSQALSG RLRAILQNQG N | |
| Human | 1 MEASPASGPR HKTYLCYEVE RLDNGTSVKM DQHRGFLHNQ AKNLLCGFYG | 9 |
| APOBEC3A | 51 RHAELRFLDL VPSLQLDPAQ IYRVTWFISW SPCFSAGCAG RVRAFLQENT | |
| isoform b | 101 HVRLRIFAAR IYDYDPLYKE ALQMLRDAGA QVSIMTYDEF KHCWDTFVDH | |
| W86A | 151 QGCPFQPWDG LDEHSQALSG RLRAILQNQG N | |
| Human | 1 MEASPASGPR HKTYLCYEVE RLDNGTSVKM DQHRGFLHNQ AKNLLCGFYG | 10 |
| APOBEC3A | 51 RHAELRFLDL VPSLQLDPAQ IYRVTWFISW SPCFSWGCAG EVRAFLQENT | |
| isoform b | 101 HVRLRIFAAR IYYYDPLYKE ALQMLRDAGA QVSIMTYDEF KHCWDTFVDH | |
| D113Y | 151 QGCPFQPWDG LDEHSQALSG RLRAILQNQG N | |
| Human | 1 MEASPASGPR HLMDPHIFTS NFNNGIGRHK TYLCYEVERL DNGTSVKMDQ | 22 |
| APOBEC3A | 51 HRGFLHNQAK NLLCGFYGRH AELRFLDLVP SLQLDPAQIY RVTWFISWSP | |
| isoform a | 101 CFSWGCAGEV RAFLQENTHV RLRIFAARIF EDDPLYKEAL QMLRDAGAQV | |
| Y130F − D131E − | 151 SIMTYDEFKH CWDTFVDHQG VFPQPWDGLD EHSQALSGRL RAILQNQGN | |
| Y132D | | |
| Human | 1 MEASPASGPR HLMDPHIFTS NFNNGIGRHK TYLCYEVERL DNGTSVKMDQ | 23 |
| APOBEC3A | 51 HRGFLHNQAK NLLCGFYGRH AELRFLDLVP SLQLDPAQIY RVTWFISWSP | |
| isoform a | 101 CFSWGCAGEV RAFLQENTHV RLRIFAARIF YDDPLYKEAL QMLRDAGAQV | |
| Y130F − D131Y − | 151 SIMTYDEFKH CWDTFVDHQG CPFQPWDGLD EHSQALSGRL RAILQNQGN | |
| Y132D | | |
| Human | 1 MEASPASGPR HLMDPHIFTS NFNNGIGRHK TYLCYEVERL DNGTSVKMDQ | 24 |
| APOBEC3A | 51 HRGFLHNQAK NLLCGFYGRH AELRDLDLVP SLQLDPAQIY RVTWFISYSP | |
| isoform a | 101 CFSWGCAGEV RAFLQENTHV RLRIFAARIY DYDPLYKEAL QMLRDAGAQV | |
| W98Y | 150 SIMTYDEFKH CWDTFVDHQG CPFQPWDGLD EHSQALSGRL RAILQNQGN | |
| Human | 1 MEASPASGPR HLMDPHIFTS NFNNGIGRHK TYLCYEVERL DNGTSVKMDQ | 25 |
| APOBEC3A | 51 HRGFLHNQAK NLLCGFYGRH AELRFLDLVP SLQLDPAQIY RVTWFISWSP | |
| isoform a | 101 VFSWGCAGEV RAFLQENTHV RLRIFAARIY DYDYLYKEAL QMLRDAGAQV | |
| P134Y | 151 SIMTYDEFKH CWDTFVDHQG CPFQPWDGLD EHSQALSGRL RAILQNQGN | |
| Human | 1 MEASPASGPR HLMDPHIFTS NFNNGIGRHK TYLCYEVERL DNGTSVKMDQ | 26 |
| APOBEC3A | 51 HRGFLHNQAK NLLCGFYGRH AELRFLDLVP SLQLDPAQIY RVTWFISYSP | |
| isoform a | 101 CFSAGCAGEV RAFLQENTHV RLRIFAARIY DYDPLYKEAL QMLRDAGAQV | |
| W98Y + W104A | 151 SIMTYDEFKH CWDTFVDHQG CPFQPWDGLD EHSQALSGRL RAILQNQGN | |
| Human | 1 MEASPASGPR HLMDPHIFTS NFNNGIGRHK TYLCYEVERL DNGTSVKMDQ | 27 |
| APOBEC3A | 51 HRGFLHNQAK NLLCGFYGRH AELRFLDLVP SLQLDPAQIY RVTWFISYSP | |
| isoform a | 101 CFSWGCAGEV RAFLQENTHV RLRIFAARIY DYDYLYKEAL QMLRDAGAQV | |
| W98Y + P134Y | 151 SIMTYDEFKH CWDTFVDHQG VPFQPWDGLD EHSQALSGRL RAILQNQGN | |
| Human | 1 MEASPASGPR HLMDPHIFTS NFNNGIGRHK TYLCYEVERL DNGTSVKMDQ | 28 |
| APOBEC3A | 51 HRGFLHNQAK NLLCGFYGRH AELRFLDLVP SLQLDPAQIY RVTWFISWSP | |
| isoform a | 101 CFSAGCAGEV RAFLQENTHV RLRIFAARIY DYDYLYKEAL QMLRDAGAQV | |
| W104A + P134Y | 151 SIMTYDEFKH CWDTFVDHQG CPFQPWDGLD EHSQALSGRL RAILQNQGN | |
| Human | 1 MEASPASGPR HLMDPHIFTS NFNNGIGRHK TYLCYEVERL DNGTSVKMDQ | 29 |
| APOBEC3A | 51 HRGFLHNQAK NLLCGFYGRH AELRFLDLVP SLQLDPAQIY RVTWFISYSP | |
| isoform a | 101 VFSAGCAGEV RAFLQENTHV RLRIFAARIF DYDPLYKEAL QMLRDAGAQV | |
| W98Y + W104A + | 151 SIMTYDEFKH CWDTFVDHQG CPFQPWDGLD EHSQALSGRL RAILQNQGN | |
| Y130F | | |
| Human | 1 MEASPASGPR HLMDPHIFTS NFNNGIGRHK TYLCYEVERL DNGTSVKMDQ | 30 |
| APOBEC3A | 51 HRGFLHNQAK NLLCGFYGRH AELRFLDLVP SLQLDPAQIY RVTWFISYSP | |
| isoform a | 101 CFSAGCAGEV RAFLQENTHV RLRIFAARIY DDDPLYKEAL QMLRDAGAQV | |
| W98Y + W104A + | 151 SIMTYDEFKH CWDTFVDHQG VPFQPWDGLD EHSQALSGRL RAILQNQGN | |
| Y132D | | |
| Human | 1 MEASPASGPR HLMDPHIFTS NFNNGIGRHK TYLCYEVERL DNGTSVKMDQ | 31 |
| APOBEC3A | 51 HRGFLHNQAK NLLCGFYGRH AELRFLDLVP SLQLDPAQIY RVTWFISWSP | |
| isoform a | 101 VFSAGCAGEV RAFLQENTHV RLRIFAARIF DYDYLYKEAL QMLRDAGAQV | |
| W104A + Y130F + | 151 SIMTYDEFKH CWDTFVDHQG CPFQPWDGLD EHSQALSGRL RAILQNQGN | |
| P134Y | | |
| Human | 1 MEASPASGPR HLMDPHIFTS NFNNGIGRHK TYLCYEVERL DNGTSVKMDQ | 32 |
| APOBEC3A | 51 HRGFLHNQAK NLLCGFYGRH AELRFLDLVP SLQLDPAQIY RVTWFISWSP | |
| isoform a | 101 CFSAGCAGEV RAFLQENTHV RLRIFAARIY DDDYLYKEAL QMLRDAGAQV | |
| W104A + Y132D + | 151 SIMTYDEFKH CWDTFVDHQG CPFQPWDGLD EHSQALSGRL RAILQNQGN | |
| P134Y | | |
| Human | 1 MEASPASGPR HLMDPHIFTS NFNNGIGRHK TYLCYEVERL DNGTSVKMDQ | 33 |
| APOBEC3A | 51 HRGFLHNQAK NLLCGFYGRH AELRFLDLVP SLQLDPAQIY RVTWFISWSP | |
| isoform a | 101 CFSAGCAGEV RAFLQENTHV RLRIFAARIF DYDPLYKEAL QMLRDAGAQV | |
| W104A + Y130F | 151 SIMTYDEFKH CWDTFVDHQG CPFQPWDGLD EHSQALSGRL RAILQNQGN | |
| Human | 1 MEASPASGPR HLMDPHIFTS NFNNGIGRHK TYLCYEVERL DNGTSVKMDQ | 34 |
| APOBEC3A | 51 HRGFLHNQAK NLLCGFYGRH AELRFLDLVP SLQLDPAQIY RVTWFISWSP | |
| isoform a | 101 CFSAGCAGEV RAFLQENTHV RLRIFAARIY DDDPLYKEAL QMLRDAGAQV | |
| W104A + Y132D | 151 SIMTYDEFKH CWDTFVDHQG CPFQPWDGLD EHSQALAGRL RAILQNQGN | |
| Human | 1 MEASPASGPR HKTYLCYEVE RLDNGTSVKM DQHRGFLHNQ AKNLLCGFYG | 35 |
| APOBEC3A | 51 RHAELRFLDL VPSLQLDPAQ IYRVTWFISY SPCFSWGCAG RVRAFLQENT | |
| isoform b W80Y | 101 HVRLRIFAAR IYDYDPLYKE ALQMLRDAGA QVSIMTYDEF KHCWDTFVDH | |
| | 151 QGCPFQPWDG LDEHSQALSG RLRAILQNQG N | |
| Human | 1 MEASPASGPR HKTYLCYEVE RLDNGTSVKM DQHRGFLHNQ AKNLLCGFYG | 36 |
| APOBEC3A | 51 RHAELRFLDL VPSLQLDPAQ IYRVTWFISW SPCFSWGCAG RVRAFLQENT | |
| isoform b P116Y | 101 HVRLRIFAAR IYDYDYLYKE ALQMLRDAGA QVSIMTYDEF KHCWDTFVDH | |
| | 151 QGCPFQPWDG LDEHSQALSG RLRAILQNQG N | |

In some embodiments, the APOBEC3A in the fusion protein of the present disclosure is human isoform a or a sequence having at least 70%, 75%, 80%, 85%, 90%, 95%, 98%, 99% of sequence identity to isoform a. In some embodiments, the APOBEC3A in the fusion protein of the present disclosure is human isoform b or a sequence having at least 70%, 75%, 80%, 85%, 90%, 95%, 98%, 99% of sequence identity to isoform b. In some embodiments, the APOBEC3A in the fusion protein of the present disclosure is rat APOBEC3 or a sequence having at least 70%, 75%, 80%, 85%, 90%, 95%, 98%, 99% of sequence identity to the rat APOBEC3. In some embodiments, the APOBEC3A in the fusion protein of the present disclosure is mouse APOBEC3 or a sequence having at least 70%, 75%, 80%, 85%, 90%, 95%, 98%, 99% of sequence identity to the mouse APOBEC3. In some embodiments, the sequence retains the cytidine deaminase activity.

In some embodiments, the APOBEC3A includes a Y130F mutation, according to residue numbering in SEQ ID NO:1 (the numbering would be different in human isoform b and rat and mouse sequences, but can readily converted). In some embodiments, the APOBEC3A includes a Y132D mutation, according to residue numbering in SEQ ID NO:1. In some embodiments, the APOBEC3A includes a W104A mutation, according to residue numbering in SEQ ID NO:1. In some embodiments, the APOBEC3A includes a D131Y mutation, according to residue numbering in SEQ ID NO:1. In some embodiments, the APOBEC3A includes a D131E mutation, according to residue numbering in SEQ ID NO:1. In some embodiments, the APOBEC3A includes a W98Y mutation, according to residue numbering in SEQ ID NO:1. In some embodiments, the APOBEC3A includes a P134Y mutation, according to residue numbering in SEQ ID NO:1.

In some embodiments, the APOBEC3A includes mutations Y130F, D131E, and Y132D, according to residue numbering in SEQ ID NO:1 (the numbering would be different in human isoform b and rat and mouse sequences, but can readily converted). In some embodiments, the APOBEC3A includes mutations Y130F, D131Y, and Y132D, according to residue numbering in SEQ ID NO:1. In some embodiments, the APOBEC3A includes mutations W98Y and W104A, according to residue numbering in SEQ ID NO:1. In some embodiments, the APOBEC3A includes mutations W98Y and P134Y, according to residue numbering in SEQ ID NO:1. In some embodiments, the APOBEC3A includes mutations W104A and P134Y, according to residue numbering in SEQ ID NO:1. In some embodiments, the APOBEC3A includes mutations W98Y, W104A, and Y130F, according to residue numbering in SEQ ID NO:1. In some embodiments, the APOBEC3A includes mutations W98Y, W104A, and Y132D, according to residue numbering in SEQ ID NO:1. In some embodiments, the APOBEC3A includes mutations W104A, Y130F, and P134Y, according to residue numbering in SEQ ID NO:1. In some embodiments, the APOBEC3A includes mutations W104A, Y132D, and P134Y, according to residue numbering in SEQ ID NO:1. In some embodiments, the APOBEC3A includes mutations W104A and Y130F, according to residue numbering in SEQ ID NO:1. In some embodiments, the APOBEC3A includes mutations W104A and Y132D, according to residue numbering in SEQ ID NO:1.

Example APOBEC3A sequences are shown in SEQ ID NO:1-10 and 22-36.

The APOBEC3A protein can allow further modifications, such as addition, deletion and/or substitutions, at other amino acid locations as well. Such modifications can be substitution at one, two or three or more positions. In one embodiment, the modification is substitution at one of the positions. Such substitutions, in some embodiments, are conservative substitutions. In some embodiments, the modified APOBEC3A protein still retains the cytidine deaminase activity. In some embodiments, the modified APOBEC3A protein retains the mutations tested in the experimental examples.

In various embodiments, the APOBEC3A can be substituted with another deaminase such as A3B (APOBEC3B), A3C (APOBEC3C), A3D (APOBEC3D), A3F (APOBEC3F), A3G (APOBEC3G), A3H (APOBEC3H), A3 (APOBEC3), and AID (AICDA).

In some embodiments, provided is a fusion protein comprising a first fragment comprising an apolipoprotein B mRNA editing enzyme catalytic subunit 3B (APOBEC3B) and a second fragment comprising a clustered regularly interspaced short palindromic repeats (CRISPR)-associated (Cas) protein. In some embodiments, provided is a fusion protein comprising a first fragment comprising an apolipoprotein B mRNA editing enzyme catalytic subunit 3C (APOBEC3C) and a second fragment comprising a clustered regularly interspaced short palindromic repeats (CRISPR)-associated (Cas) protein. In some embodiments, provided is a fusion protein comprising a first fragment comprising an apolipoprotein B mRNA editing enzyme catalytic subunit 3D (APOBEC3D) and a second fragment comprising a clustered regularly interspaced short palindromic repeats (CRISPR)-associated (Cas) protein. In some embodiments, provided is a fusion protein comprising a first fragment comprising an apolipoprotein B mRNA editing enzyme catalytic subunit 3F (APOBEC3F) and a second fragment comprising a clustered regularly interspaced short palindromic repeats (CRISPR)-associated (Cas) protein. In some embodiments, provided is a fusion protein comprising a first fragment comprising an apolipoprotein B mRNA editing enzyme catalytic subunit 3G (APOBEC3G) and a second fragment comprising a clustered regularly interspaced short palindromic repeats (CRISPR)-associated (Cas) protein. In some embodiments, provided is a fusion protein comprising a first fragment comprising an apolipoprotein B mRNA editing enzyme catalytic subunit 3H (APOBEC3H) and a second fragment comprising a clustered regularly interspaced short palindromic repeats (CRISPR)-associated (Cas) protein. In some embodiments, provided is a fusion protein comprising a first fragment comprising an apolipoprotein B mRNA editing enzyme catalytic subunit 3 (APOBEC3) and a second fragment comprising a clustered regularly interspaced short palindromic repeats (CRISPR)-associated (Cas) protein. In some embodiments, provided is a fusion protein comprising a first fragment comprising an apolipoprotein B mRNA editing enzyme catalytic subunit AID (AICDA) and a second fragment comprising a clustered regularly interspaced short palindromic repeats (CRISPR)-associated (Cas) protein.

In some embodiments, the APOBEC protein is a human protein. In some embodiments, the APOBEC protein is a mouse or rat protein. Some example APOBEC proteins are listed in the table below.

| | Example | |
|---|---|---|
| Deaminase | version | NCBI Accession Nos. |
| A3B (APOBEC3B) | hA3B (human) | NP_001257340, NP_004891 |
| A3C (APOBEC3C) | hA3C (human) | NP_055323 |
| A3D (APOBEC3D) | hA3D (human) | NP_689639, NP_001350710 |
| A3F (APOBEC3F) | hA3F (human) | NP_660341, NP_001006667 |
| A3G (APOBEC3G) | hA3G (human) | NP_068594, NP_001336365, |
| | | NP_001336366, NP_001336367 |
| A3H (APOBEC3H) | hA3H (human) | NP_001159474, NP_001159475, |
| | | NP_001159476, and NP_861438 |
| A1 (APOBEC1) | hA1 (human) | NP_001291495, NP_001635, |
| | | NP_005880 |
| | mA1 (mouse) | NP_001127863, NP_112436 |
| A3 (APOBEC3) | mA3 (mouse) | NP_001153887, NP_001333970, |
| | | NP_084531 |
| AID (AICDA) | hAID (human) | NP_001317272, NP_065712 |
| | mAID (mouse) | NP_033775 |
| | cAICDA | NP_001187114 |
| | (channel | |
| | catfish) | |

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Non-limiting examples of conservative amino acid substitutions are provided in the table below, where a similarity score of 0 or higher indicates conservative substitution between the two amino acids.

TABLE A
Amino Acid Similarity Matrix

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

TABLE B
Conservative Amino Acid Substitutions

| For Amino | |
|---|---|
| Acid | Substitution With |
| Alanine | D-Ala, Gly, Aib, β-Ala, L-Cys, D-Cys |
| Arginine | D-Arg, Lys, D-Lys, Orn D-Orn |
| Asparagine | D-Asn, Asp, D-Asp, Glu, D-Glu Gln, D-Gln |
| Aspartic Acid | D-Asp, D-Asn, Asn, Glu, D-Glu, Gln, D-Gln |
| Cysteine | D-Cys, S-Me-Cys, Met, D-Met, Thr, D-Thr, |
| | L-Ser, D-Ser |
| Glutamine | D-Gln, Asn, D-Asn, Glu, D-Glu, Asp, D-Asp |
| Glutamic Acid | D-Glu, D-Asp, Asp, Asn, D-Asn, Gln, D-Gln |
| Glycine | Ala, D-Ala, Pro, D-Pro, Aib, β-Ala |
| Isoleucine | D-Ile, Val, D-Val, Leu, D-Leu, Met, D-Met |
| Leucine | Val, D-Val, Met, D-Met, D-Ile, D-Leu, Ile |
| Lysine | D-Lys, Arg, D-Arg, Orn, D-Orn |
| Methionine | D-Met, S-Me-Cys, Ile, D-Ile, Leu, D-Leu, |
| | Val, D-Val |
| Phenylalanine | D-Phe, Tyr, D-Tyr, His, D-His, Trp, D-Trp |
| Proline | D-Pro |
| Serine | D-Ser, Thr, D-Thr, allo-Thr, L-Cys, D-Cys |
| Threonine | D-Thr, Ser, D-Ser, allo-Thr, Met, D-Met, |
| | Val, D-Val |
| Tyrosine | D-Tyr, Phe, D-Phe, His, D-His, Trp, D-Trp |
| Valine | D-Val, Leu, D-Leu, Ile, D-Ile, Met, D-Met |

The term “clustered regularly interspaced short palindromic repeats (CRISPR)-associated (Cas) protein” or simply “Cas protein” refers to RNA-guided DNA endonuclease enzymes associated with the CRISPR (Clustered Regularly Interspaced Short Palindromic Repeats) adaptive immunity system in Streptococcus pyogenes, as well as other bacteria. Non-limiting examples of Cas proteins include Streptococcus pyogenes Cas9 (SpCas9), Staphylococcus aureus Cas9 (SaCas9), Acidaminococcus sp. Cas12a (Cpf1), Lachnospiraceae bacterium Cas12a (Cpf1), Francisella novicida Cas12a (Cpf1). Additional examples are provided in Komor et al., “CRISPR-Based Technologies for the Manipulation of Eukaryotic Genomes,” Cell. 2017 Jan. 12; 168(1-2):20-36.

Example Cas proteins include SpCas9, FnCas9, St1Cas9, St3Cas9, NmCas9, SaCas9, AsCpf1, LbCpf1, FnCpf1, VQR SpCas9, EQR SpCas9, VRER SpCas9, RHA FnCas9, KKH SaCas9, NmeCas9, StCas9, CjCas9, AsCpf1, FnCpf1, SsCpf1, PcCpf1, BpCpf1, CmtCpf1, LiCpf1, PmCpf1, Pb3310Cpf1, Pb4417Cpf1, BsCpf1, EeCpf1, BhCas12b, AkCas12b, EbCas12b, LsCas12b, RfCas13d, LwaCas13a, PspCas13b, PguCas13b, RanCas13b, CasX, CasY and those provided in Table C below.

TABLE C
Example Cas Proteins

| Cas protein types | Cas proteins |
|---|---|
| Cas9 proteins | Cas 9 from Streptococcus pyogenes (SpCas9) |
| | Cas9 from Staphylococcus aureus (SaCas9) |
| | Cas9 from Neisseria meningitidis (NmeCas9) |
| | Cas9 from Streptococcus thermophilus (StCas9) |
| | Cas9 from Campylobacter jejuni (CjCas9) |
| Cas12a (Cpf1) proteins | Cas12a (Cpf1) from Lachnospiraceae bacterium Cas12a (LbCpf1) |
| | Cas12a (Cpf1) from Acidaminococcus sp BV3L6 (AsCpf1) |
| | Cas12a (Cpf1) from Francisella novicida sp BV3L6 (FnCpf1) |
| | Cas12a (Cpf1) from Smithella sp SC K08D17 (SsCpf1) |
| | Cas12a (Cpf1) from Porphyromonas crevioricanis (PcCpf1) |
| | Cas12a (Cpf1) from Butyrivibrio proteoclasticus (BpCpf1) |
| | Cas12a (Cpf1) from Candidatus Methanoplasma termitum (CmtCpf1) |
| | Cas12a (Cpf1) from Leptospira inadai (LiCpf1) |
| | Cas12a (Cpf1) from Porphyromonas macacae (PmCpf1) |
| | Cas12a (Cpf1) from Peregrinibacteria bacterium GW2011 WA2 33 10 |
| | (Pb3310Cpf1) |
| | Cas12a (Cpf1) from Parcubacteria bacterium GW2011 GWC2 44 17 |
| | (Pb4417Cpf1) |
| | Cas12a (Cpf1) from Butyrivibrio sp. NC3005 (BsCpf1) |
| | Cas12a (Cpf1) from Eubacterium eligens (EeCpf1) |
| Cas12b (C2c1) proteins | Cas12b (C2c1) Bacillus hisashii (BhCas12b) |
| | Cas12b (C2c1) Bacillus hisashii with a gain-of-function mutation (see, |
| | e.g., Strecker et al., Nature Communications 10 (article 212) (2019) |
| | Cas12b (C2c1) Alicyclobacillus kakegawensis (AkCas12b) |
| | Cas12b (C2c1) Elusimicrobia bacterium (EbCas12b) |
| | Cas12b (C2c1) Laceyella sediminis (Ls) (LsCas12b) |
| Cas13 proteins | Cas13d from Ruminococcus flavefaciens XPD3002 (RfCas13d) |
| | Cas13a from Leptotrichia wadei (LwaCas13a) |
| | Cas13b from Prevotella sp. P5-125 (PspCas13b) |
| | Cas13b from Porphyromonas gulae (PguCas13b) |
| | Cas13b from Riemerella anatipestifer (RanCas13b) |
| Engineered Cas proteins | Nickases (mutation in one nuclease domain) |
| | Catalytically inactive mutant (dCas; mutations in both of the nuclease |
| | domains) |
| | Enhanced variants with improved specificity (see, e.g., Chen et al., |
| | Nature, 550, 407-410 (2017) |

In some embodiments, the Cas protein is a mutant of protein selected from the above, wherein the mutant retains the DNA-binding capability but does not introduce double strand DNA breaks.

For example, it is known that in SpCas9, residues Asp10 and His840 are important for Cas9's catalytic (nuclease) activity. When both residues are mutated to Ala, the mutant loses the nuclease activity. In another embodiment, only the Asp10Ala mutation is made, and such a mutant protein cannot generate a double strand break; rather, a nick is generated on one of the strands. Such a mutant is also referred to as a Cas9 nickase. A non-limiting example of a Cas9 nickase is provided is SEQ ID NO: 11. Non-limiting example of a Cas12a nickase are provided is SEQ ID NO:37-39. Cas proteins also encompass mutants of known Cas proteins that have certain sequence identity (e.g., 70%, 75%, 80%, 85%, 90%, 95%, 98%, 99% or more). In some embodiments, the Cas protein retains the catalytic (nuclease) activity.

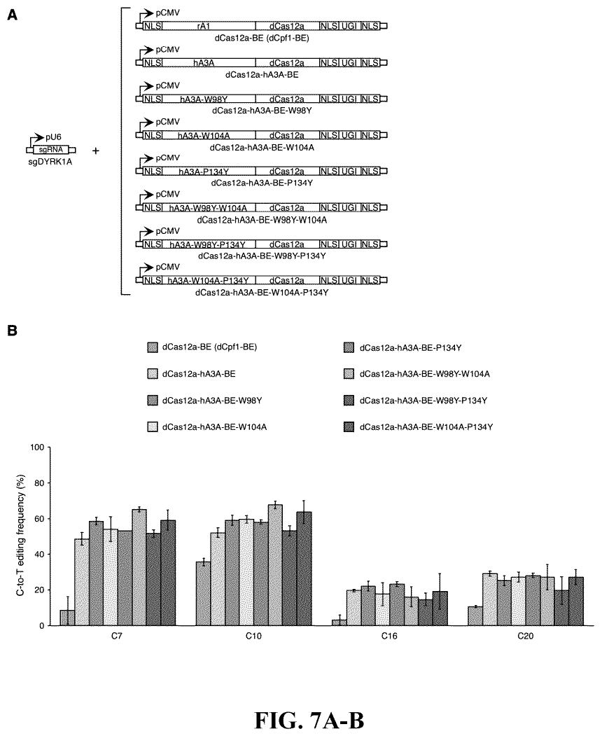

In some embodiments, the Cas protein in a fusion protein of the present disclosure is a Cas12a (Cpf1, CRISPR-associated endonuclease in Prevotella and Francisella 1) protein. In conventional base editors, Cas9 is the commonly used DNA endonuclease. The Cas12a (Cpf1) has the advantage of recognizing A/T rich sequence when used together with APOBEC1 in base editors. In another surprising discovery of the present disclosure, when APOBEC1 was replaced with A3A, the editing efficiency was greatly increased (see, e.g., Examples 3-5 and FIGS. 7B, 9B and 11B). Yet, the editing efficiency of such a Cas12a-A3A can be further increased when the A3A includes a few tested mutations (Examples 3-5 and FIGS. 7B, 9B and 11B) and the editing window such a Cas12a-A3A can be narrowed to achieve more precise editing when even more tested mutations are included in A3A (Examples 3-5 and FIGS. 8B, 10B and 12B).

In some embodiments, therefore, provided is a fusion protein comprising a first fragment comprising an apolipoprotein B mRNA editing enzyme catalytic subunit 3A (APOBEC3A) and a second fragment comprising a CRISPR-associated endonuclease in Prevotella and Francisella 1 (Cpf1). Examples of APOBEC3A, as well as its alternatives (e.g., A3B (APOBEC3B), A3C (APOBEC3C), A3D (APOBEC3D), A3F (APOBEC3F), A3G (APOBEC3G), A3H (APOBEC3H), A3 (APOBEC3), or AID (AICDA)) and biological equivalents (homologues) have been disclosed above. Non-limiting example fusion sequences are provided in SEQ ID NO:40-50.

In some embodiments, the fusion protein further comprises a uracil glycosylase inhibitor (UGI). A non-limiting example of UGI is found in Bacillus phage AR9 (YP_009283008.1). In some embodiments, the UGI comprises the amino acid sequence of SEQ ID NO:12 or has at least at least 90% sequence identity to SEQ ID NO:12 and retains the uracil glycosylase inhibition activity.

In some embodiments, the UGI is not fused to the fusion protein, but rather is provided separately (free UGI, not fused to a Cas protein or a cytosine deaminase) when the fusion protein is used for genomic editing. In some embodiments, the free UGI is provided with the fusion protein which also includes a UGI portion.

Preferably, a peptide linker is provided between each of the fragments in the fusion protein. In some embodiments, the peptide linker has from 1 to 100 amino acid residues (or 3-20, 4-15, without limitation). In some embodiments, at least 10%, 20%, 30%, 40%, 50%, 60%, 70%, 80% or 90% of the amino acid residues of peptide linker are amino acid residues selected from the group consisting of alanine, glycine, cysteine, and serine. In some embodiments, the peptide linker has an amino acid sequence of SEQ ID NO:13 or 14.

The APOBEC3A, Cas protein, and UGI can be arranged in any manner. However, in a preferred embodiment, APOBEC3A is placed at the N-terminal side of the Cas protein. In one embodiment, the Cas protein is placed at the N-terminal side of the UGI.

In some embodiments, the fusion protein further comprises a nuclear localization sequence such as SEQ ID NO:15.

Non-limiting examples of fusion proteins include those having an amino acid sequence selected from the group consisting of SEQ ID NO:16-20.

TABLE 2
Additional Sequences

| Name | Sequence | SEQ ID NO: |
|---|---|---|
| Cas9-Nickase | 1 MYPYDVPDYA SPKKKRKVEA SDKKYSIGLA IGTNSVGWAV ITDEYKVPSK | 11 |
| | 51 KFKVLGNTDR HSIKKNLIGA LLFDSGETAE ATRLKRTARR RYTRRKNRIC | |
| | 101 YLQEIFSNEM AKVDDSFFHR LEESFLVEED KKHERHPIFG NIVDEVAYHE | |
| | 151 KYPTIYHLRK KLVDSTDKAD LRLIYLALAH MIKFRGHFLI EGDLNPDNSD | |
| | 201 VDKLFIQLVQ TYNQLFEENP INASGVDAKA ILSARLSKSR RLENLIAQLP | |
| | 251 GEKKNGLFGN LIALSLGLTP NFKSNFDLAE DAKLQLSKDT YDDDLDNLLA | |
| | 301 QIGDQYADLF LAAKNLSDAI LLSDILRVNT EITKAPLSAS MIKRYDEHHQ | |
| | 351 DLTLLKALVR QQLPEKYKEI FFDQSKNGYA GYIDGGASQE EFYKFEIPIL | |
| | 401 EKMDGTEELL VKLNREDLLR KQRTFDNGSI PHQIHLGELH AILRRQEDFY | |
| | 451 PFLKDNREKI EKILTFRIPY YVGPLARGNS RFAWMTRKSE ETITPWNFEE | |
| | 501 VVDKGASAQS FIERMTNFDK NLPNEKVLPK HSLLYEYFTV YNELTKVKYV | |
| | 551 TEGMRKPAFL SGEQKKAIVD LLFKTNRKVT VKQLKEDYFK KIECFDSVEI | |
| | 601 SGVEDRFNAS LGTYHDLLKI IKDKDFLDNE ENEDILEDIV LTLTLFEDRE | |
| | 651 MIEERLKTYA HLFDDKVMKQ KLRRRYTGWG RLSRKLINGI RDKQSGKTIL | |
| | 701 DFLKSDGFAN RNFMQLIHDD SLTFKEDIQK AQVSGQGDSL HEHIANLAGS | |
| | 751 PAIKKGILQT VKVVDELVKV MGRHKPENIV IEMARENQTT QKGQKNSRER | |
| | 801 MKRIEEGIKE LGSQILKEHP VENTQLQNEK LYLYYLQNGR DMYVDQELDI | |
| | 851 NRLSDYDVDH IVPQSFLKDD SIDNKVLTRS DKNRGKSDNV PSEEVVKKMK | |
| | 901 NYWRQLLNAK LITQRKFDNL TKAERGGLSE LDKAGFIKRQ LVETRQITKH | |
| | 951 VAQILDSRMN TKYDENDKLI REVKVITLKS KLVSDFRKDF QFYKVREINN | |
| | 1001 YHHAHDAYLN AVVGTALIKK YPKLESEFVY GDYKVYDVRK MIAKSEQEIG | |
| | 1051 KATAKYFFYS NIMNFFKTEI TLANGEIRKR PLIETNGETG EIVWDKGRDF | |
| | 1101 ATVRKVLSMP QVNIVKKTEV QTGGFSKESI LPKRNSDKLI ARKKDWDPKK | |
| | 1151 YGGFDSPTVA YSVLVVAKVE KGKSKKLKSV KELLGITIME RSSFEKNPID | |
| | 1201 FLEAKGYKEV KKDLIIKLPK YSLFELENGR KRMLASAGEL QKGNELALPS | |
| | 1251 KYVNFLYLAS HYEKLKGSPE DNEQKQLFVE QHKHYLDEII EQISEFSKRV | |
| | 1301 ILADANLDKV LSAYNKHRDK PIREQAENII HLFTLTNLGA PAAFKYFDTT | |
| | 1351 IDRKRYTSTK EVLDATLIHQ SITGLYETRI DLSQLGGDSP KKKRKVEAS | |
| Uracil-DNA- | 1 TNLSDIIEKE TGKQLVIQES ILMLPEEVEE VIGNKPESDI LVHTAYDEST | 12 |
| glycosylase | 51 DENVMLLTSD APEYKPWALV IQDSNGENKI KML | |
| inhibitor (UGI) | | |
| Linker 1 | 1 SGSETPGTSE SATPES | 13 |
| Linker 2 | 1 SGGS | 14 |
| Nuclear | 1 PKKKRKV | |
| localization | | |
| sequence | | |
| Fusion protein 1 | 1 MEASPASGPR HLMDPHIFTS NFNNGIGRHK TYLCYEVERL DNGTSVKMDQ | 16 |
| | 51 HRGFLHNQAK NLLCGFYGRH AELRFLDLVP SLQLDPAQIY RVTWFISWSP | |
| | 101 CFSWGCAGEV RAFLQENTHV RLRIFAARIY DYDPLYKEAL QMLRDAGAQV | |
| | 151 SIMTYDEFKH CWDTFVDHQG CPFQPWDGLD EHSQALSGRL RAILQNQGNS | |
| | 201 GSETPGTSES ATPESDKKYS IGLAIGTNSV GWAVITDEYK VPSKKFKVLG | |
| | 251 NTDRHSIKKN LIGALLFDSG ETAEATRLKR TARRRYTRRK NRICYLQEIF | |
| | 301 SNEMAKVDDS FFHRLEESFL VEEDKKHERH PIFGNIVDEV AYHEKYPTIY | |
| | 351 HLRKKLVDST DKADLRLIYL ALAHMIKFRG HFLIEGDLNP DNSDVDKLFI | |
| | 401 QLVQTYNQLF EENPINASGV DAKAILSARL SKSRRLENLI AQLPGEKKNG | |
| | 451 LFGNLIALSL SLTPNFKSNF DLAEDAKLQL SKDTYDDDLD NLLAQIGDQY | |
| | 501 ADLFLAAKNL SDAILLSDIL RVNTEITKAP LSASMIKRYD EHHQDLTLLK | |
| | 551 ALVRQQLPEK YKEIFFDQSK NGYAGYIDGG ASQEEYFKFI KPILEKMDGT | |
| | 601 EELLVKLNRE DLLRKQRTFD NGSIPHQIHL GELHAILRRQ EDFYPFLKDN | |
| | 651 REKIEKILTF RIPYYVGPLA RGNSRFAWMT RKSEETITPW NFEEVVDKGA | |
| | 701 SAQSFIERMT NFDKNLPNEK VLPKHSLLYE YFTVYNELTK VKYVTEGMRK | |
| | 751 PAFLSGEQKK AIVDLLFKTN RKVTVKQLKE DYFKKIECFD SVEISGVEDR | |
| | 801 FNASLGTYHD LLKIIKDKDF LDNEENEDIL EDIVLTLTLF EDREMIEERL | |
| | 851 KTYAHLFDDK VMKQLKRRRY TGWGRLSRKL INGIRDKQSG KTILDFLKSD | |
| | 901 GFANRNFMQL IHDDSLTFKE IDQKAQVSGQ GDSLHEHIAN LAGSPAIKKG | |
| | 951 ILQTVKVVDE LVKVMGRHKP ENIVIEMARE NQTTQKGQKN SRERMKRIEE | |
| | 1001 GIKELGSQIL HEKPVENTQL QNEKLYLYYL QNGRDMYVDQ ELDINRLSDY | |
| | 1051 DVDHIVPQSF LKDDSIDNKV LTRSDKNRGK SDNVPSEEVV KKMKNYWRQL | |
| | 1101 LNAKLITQRK FDNLTKAERG GLSELDKAGF IKRQLVETRQ ITKHVAQILD | |
| | 1151 SRMNTKYDEN DKLIREVKVI TLKSKLVSDF RKDFQFYKVR EINNYHHAHD | |
| | 1201 AYLNAVVGTA LIKKYPKLES EFVYGDYKVY DVRKMIAKSE QEIGKATAKY | |
| | 1251 FFYSNIMNFF KTEITLANGE IRKRPLIETN GETGEIVWDK GRDFATVRKV | |
| | 1301 LSMPQVNIVK KTEVQTGGFS KESILPKRNS DKLIARKKDW DPKKYGGFDS | |
| | 1351 PTVAYSVLVV AKVEKGKSKK LKSVKELLGI TIMERSSFEK NPIDFLEAKG | |
| | 1401 YKEVKKDLII KLPKYSLFEL ENGRKRMLAS AGELQKGNEL ALPSKYVNFL | |
| | 1451 YLASHYEKLK GSPEDNEQKQ LFVEQHKHYL DEIIEQISEF SKRVILADAN | |
| | 1501 LDKVLSAYNK HRDKPIREQA ENIIHLFTLT NLGAPAAFKY FDTTIDRKRY | |
| | 1551 TSTKEVLDAT LIHQSITGLY ETRIDLSQLG GDSGGSTNLS DIIEKETGKQ | |
| | 1601 LVIQESILML PEEVEEVIGN KPESDILVHT AYDESTDENV MLLTSDAPEY | |
| | 1651 KPWALVIQDS NGENKIKMLS GGSPKKKRKV | |
| Fusion protein 2 | 1 MEASPASGPR HLMDPHIFTS NFNNGIGRHK TYLCYEVERL DNGTSVKMDQ | 17 |
| (Y130F) | 51 HRGFLHNQAK NLLCGFYGRH AELRFLDLVP SLQLDPAQIY RVTWFISWSP | |
| | 101 CFSWGCAGEV RAFLQENTHV RLRIFAARIF DYDPLYKEAL QMLRDAGAQV | |
| | 151 SIMTYDEFKH VWDTFVDHQG CPFQPWDGLD EHSQALSGRL RAILQNQGNS | |
| | 201 GSETPGTSES ATPESDKKYS IGLAIGTNSV GWAVITDEYK VPSKKFKVLG | |
| | 251 NTDRHSIKKN LIGALLFDSG ETAEATRLKR TARRRYTRRK NRICYLQEIF | |
| | 301 SNEMAKVDDS FFHRLEESFL VEEDKKHERH PIFGNIVDEV AYHEKYPTIY | |
| | 351 HLRKKLVDST DKADLRLIYL ALAHMIKFRG HFLIEGDLNP DNSDVDKLFI | |
| | 401 QLVQTYNQLF EENPINASGV DAKAILSARL SKSRRLENLI AQLPGEKKNG | |
| | 451 LFGNLIALSL GLTPNFKSNF DLAEDAKLQL SKDTYDDDLD NLLAQIGDQY | |
| | 501 ADLFLAAKNL SDAILLSDIL RVNTEITKAP LSASMIKRYD EHHQDLTLLK | |
| | 551 ALVRQQLPEK YKEIFFDQSK NGYAGYIDGG ASQEEFYKFI KIPLEKMDGT | |
| | 601 EELLVKLNRE DLLRKQRTFD NGSIPHQIHL GELHAILRRQ EDFYPFLKDN | |
| | 651 REKIEKILTF RIPYYVGPLA RGNSRFAWMT RKSEETITPW NFEEVVDKGA | |
| | 701 SAQSFIERMT NFDKNLPNEK VLPKHSLLYE YFTVYNELTK VKYVTEGMRK | |
| | 751 PAFLSGEQKK QIVDLLFKTN RKVTVKQLKE DYFKKIECFD SVEISGVEDR | |
| | 801 FNASLGTYHD LLKIIKDKDF LDNEENEDIL EDIVLTLTLF EDREMIEERL | |
| | 851 KTYAHLFDDK VMKQLKRRRY TGWGRLSRKL INGIRDKQSG KTILDFLKSD | |
| | 901 GFANRNFMQL IHDDSLTFKE DIQKAQVSGQ GDSLHEHIAN LAGSPAIKKG | |
| | 951 ILQTVKVVDE KVKVMGRHKP ENIVIEMARE NQTTQKGQKN SRERMKRIEE | |
| | 1001 GIKELGSQIL KEHPVENTQL QNEKLYLYYL QNGRDMYVDQ ELDINRLSDY | |
| | 1051 DVDHIVPQSF LKDDSIDNKV LTRSDKNRGK SDNVPSEEVV KKMKNYWRQL | |
| | 1101 LNAKLITQRK FDNLTKAERG GLSELDKAGF IKRQLVETRQ ITKHVAQILD | |
| | 1151 SRMNTKYDEN DKLIREVKVI TLKSKLVSDF RKDFQFYKVR EINNYHHAHD | |
| | 1201 AYLNAVVGTA LIKKYPKLES EFVYGDYKVY DVRKMIAKSE QEIGKATAKY | |
| | 1251 FFYSNIMNFF KTEITLANGE IRKRPLIETN GETGEIVWDK GRDFATVRKV | |
| | 1301 LSMPQVNIVK KTEVQTGGFS KESILPKRNS DKLIARKKDW DPKKYGGFDS | |
| | 1351 PTVAYSVLVV AKVEKGKSKK LKSVKELLGI TIMERSSFEK NPIDFLEAKG | |
| | 1401 YKEVKKDLII KLPKYSLFEL ENGRKRMLAS AGELQKGNEL ALPSLYVNFL | |
| | 1451 YLASHYEKLK GSPEDNEQKQ LFVEQHKHYL DEIIEQISEF SKRVILADAN | |
| | 1501 LDKVLSAYNK HRDKPIREQA ENIIHLFTLT NLGAPAAFKY FDTTIDRKRY | |
| | 1551 TSTKEVLDAT LIHQSITGLY ETRIDLSQLG GDSGGSTNLS DIIEKETGKQ | |
| | 1601 LVIQESILML PEEVEEVIGN KPESDILVHT AYDESTDENV MLLTSDAPEY | |
| | 1651 KPWALVIQDS NGENKIKMLS GGSPKKKRKV | |
| Fusion protein 3 | 1 MEASPASGPR HLMDPHIFTS NFNNGIGRHK TYLCYEVERL DNGTSVKMDQ | 18 |
| (Y132D) | 51 HRGFLHNQAK NLLCGFYGRH AELRFLDLVP SLQLDPAQIY RVTWFISWSP | |
| | 101 CFSWGCAGEV RAFLQENTHV RLRIFAARIY DDDPLYKEAL QMLRDAGAQV | |
| | 151 SIMTYDEFKH CWDTFVDHQG CPFQPWDGLD EHSQALSGRL RAILQNQGNS | |
| | 201 GSETPGTSES ATPESDKKYS IGLAIGTNSV GWAVITDEYK VPSKKFKVLG | |
| | 251 NTDRHSIKKN LIGALLFDSG ETAEATRLKR TARRRYTRRK NRICYLQEIF | |
| | 301 SNEMAKVDDS FFHRLEESFL VEEDKKHERH PIFGNIVDEV AYHEKYPTIY | |
| | 351 HLRKKLVDST DKADLRLIYL ALAHMIKFRG HFLIEGDLNP DNSDVDKLFI | |
| | 401 QLVQTYNQLF EENPINASGV DAKAILSARL SKSRRLENLI AQLPGEKKNG | |
| | 451 LFGNLIALSL GLTPNFKSNF DLAEDAKLQL SKDTYDDDLD NLLAQIGDQY | |
| | 501 ADLFLAAKNL SDAILLSDIL RVNTEITKAP LSASMIKRYD EHHQDLTLLK | |
| | 551 ALVRQQLPEK YKEIFFDQSK NGYAGYIDGG ASQEEFYKFI KPILEKMDGT | |
| | 601 EELLVKLNRE DLLRKQRTFD NGSIPHQIHL GELHAILRRQ EDFYPFLKDN | |
| | 651 REKIEKILTF RIPYYVGPLA RGNSRFAWMT RKSEETITPW NFEEVVDKGA | |
| | 701 SAQSFIERMT NFDKNLPNEK VLPKHSLLYE YFTVYNELTK VKYVTEGMRK | |
| | 751 PAFLSGEQKK AIVDLLFKTN RKVTVKQLKE DYFKKIECFD SVEISGVEDR | |
| | 801 FNASLGTYHD LLKIIKDKDF LDNEENEDIL EDIVLTLTLF EDREMIEERL | |
| | 851 KTYAHLFDDK VMKQLKRRRY TGWGRLSRKL INGIRDKQSG KTILDFLKSD | |
| | 901 GFANRNFMQL IHDDSLTFKE DIQKAQVSGQ GDSLHEHIAN LAGSPAIKKG | |
| | 951 ILQTVKVVDE LVKVMGRHKP ENIVIEMARE NQTTQKGQKN SRERMKRIEE | |
| | 1001 GIKELGSQIL KEHPVENTQL QNEKLYLYYL QNGRDMYVDQ ELDINRLSDY | |
| | 1051 DVDHIVPQSF LKDDSIDNKV LTRSDKNRGK SDNVPSEEVV KKMKNYWRQL | |
| | 1101 LNAKLITQRK FDNLTKAERG GLSELDKAGF IKRQLVETRQ ITKHVAQILD | |
| | 1151 SRMNTKYDEN DKLIREVKVI TLKSKLVSDF RKDFQFYKVR EINNYHHAHD | |
| | 1201 AYLNAVVGTA LIKKYPKLES EFVYGDYKVY DVRKMIAKSE QEIGKATAKY | |
| | 1251 FFYSNIMNFF KTEITLANGE IRKRPLIETN GETGEIVWDK GRDFATVRKV | |
| | 1301 LSMPQVNIVK KTEVQTGGFS KESILPKRNS DKLIARKKDW DPKKYGGFDS | |
| | 1351 PTVAYSVLVV AKVEKGKSKK LKSVKELLGI TIMERSSFEK NPIDFLEAKG | |
| | 1401 YKEVKKDLII KLPKYSLFEL ENGRKRMLAS AGELQKGNEL ALPSKYVNFL | |
| | 1451 YLASHYEKLK GSPEDNEQKQ LFVEQHKHYL DEIIEQISEF SKRVILADAN | |
| | 1501 LDKVLSAYNK HRDKPIREQA ENIIHLFTLT NLGAPAAFKY FDTTIDRKRY | |
| | 1551 TSTKEVLDAT LIHQSITGLY ETRIDLSQLG GDSGGSTNLS DIIEKETGKQ | |
| | 1601 LVIQESILML PEEVEEVIGN KPESDILVHT AYDESTDENV MLLTSDAPEY | |
| | 1651 KPWALVIQDS NGENKIKMLS GGSPKKKRKV | |
| Fusion protein 4 | 1 MEASPASGPR HLMDPHIFTS NFNNGIGRHK TYLCYEVERL DNGTSVKMDQ | 19 |
| (W104A) | 51 HRGFLHNQAK NLLCGFYGRH AELRFLDLVP SLQLDPAQIY RVTWFISWSP | |
| | 101 CFSAGCAGEV RAFLQENTHV RLRIFAARIY DYDPLYKEAL QMLRDAGAQV | |
| | 151 SIMTYDEFKH CWDTFVDHQG CPFQPWDGLD EHSQALSGRL RAILQNQGNS | |
| | 201 GSETPGTSES ATPESDKKYS IGLAIGTNSV GWAVITDEYK VPSKKFKVLG | |
| | 251 NTDRHSIKKN LIGALLFDSG ETAEATRLKR TARRRYTRRK NRICYLQEIF | |
| | 301 SNEMAKVDDS FFHRLEESFL VEEDKKHERH PIFGNIVDEV AYHEKYPTIY | |
| | 351 HLRKKLVDST DKADLRLIYL ALAHMIKFRG HFLIEGDLNP DNSDVDKLFI | |
| | 401 QLVQTYNQLF EENPINASGV DAKAILSARL SKSRRLENLI AQLPGEKKNG | |
| | 451 LFGNLIALSL GLTPNFKSNF DLAEDAKLQL SKDTYDDDLD NLLAQIGDQY | |
| | 501 ADLFLAAKNL SDAILLSDIL RVNTEITKAP LSASMIKRYD EHHQDLTLLK | |
| | 551 ALVRQQLPEK YEIKFFDQSK NGYAGYIDGG ASQEEFYKFI KPILEKMDGT | |
| | 601 EELLVKLNRE DLLRKQRTFD NGSIPHQIHL GELHAILRRQ EDFYPFLKDN | |
| | 651 REKIEKILTF RIPYYVGPLA RGNSRFAWMT RKSEETITPW NFEEVVDKGA | |
| | 701 SAQSFIERMT NFDKNLPNEK VLPKHSLLYE YFTVYNELTK VKYVTEGMRK | |
| | 751 PAFLSGEQKK AIVDLLFKTN RKVTVKQLKE DYFKKIECFD SVEISGVEDR | |
| | 801 FNASLGTYHD LLKIIKDKDF LDNEENEDIL EDIVLTLTLF EDREMIEERL | |
| | 851 KTYAHLFDDK VMKQLKRRRY TGWGRLSRKL INGIRDKQSG KTILDFLKSD | |
| | 901 GFANRNFMQL IHDDSLTFKE DIQKAQVSGQ GDSLHEHIAN LAGSPAIKKG | |
| | 951 ILQTVKVVDE LVKVMGHRKP ENIVIEMARE NQTTQKGQKN SRERMKRIEE | |
| | 1001 GIKELGSQIL KEHPVENTQL QNEKLYLYYL QNGRDMYVDQ ELDINRLSDY | |
| | 1051 DVDHIVPQSF LKDDSIDNKV LTRSDKNRGK SDNVPSEEVV KKMKNYWRQL | |
| | 1101 LNAKLITQRK FDNLTKAERG GLSELDKAGF IKRQLVETRQ ITKHVAQILD | |
| | 1151 SRMNTKYDEN DKLIREVKVI TLKSKLVSDF RKDFQFYKVR EINNYHHAHD | |
| | 1201 AYLNAVVGTA LIKKYPKLES EFVYGDYKVY DVRKMIAKSE QEIGKATAKY | |
| | 1251 FFYSNIMNFF KTEITLANGE IRKRPLIETN GETGEIVWDK GRDFATVRKV | |
| | 1301 LSMPQVNIVK KTEVQTGGFS KESILPKRNS DKLIARKKDW DPKKYGGFDS | |
| | 1351 PTVAYSVLVV AKVEKGKSKK LKSVKELLGI TIMERSSFEK NPIDFLEAKG | |
| | 1401 YKEVKKDLII KLPKYSLFEL ENGRKRMLAS AGELQKGNEL ALPSKYVNFL | |
| | 1451 YLASHYEKLK GSPENDEQKQ LFVEQHKHYL DEIIEQISEF SKRVILADAN | |
| | 1501 LDKVLSAYNK HRDKPIREQA ENIIHLFTLT NLGAPAAFKY FDTTIDRKRY | |
| | 1551 TSTKEVLDAT LIHQSITGLY ETRIDLSQLG GDSGGSTNLS DIIEKETGKQ | |
| | 1601 LVIQESILML PEEVEEVIGN KPESDILVHT AYDESTDENV MLLTSDAPEY | |
| | 1651 KPWALVIQDS NGENKIKMLS GGSPKKKRKV | |
| Fusion protein 5 | 1 MEASPASGPR HLMDPHIFTS NFNNGIGRHK TYLCYEVERL DNGTSVKMDQ | 20 |
| | 51 HRGFLHNQAK NLLCGFYGRH AELRFLDLVP SLQLDPAQIY RVTWFISWSP | |
| | 101 CFSWGCAGEV RAFLQENTHV RLRIFAARIY YYDPLYKEAL QMLRDAGAQV | |
| | 151 SIMTYDEFKH CWDTFVDHQG CPFQPWDGLD EHSQALSGRL RAILQNQGNS | |
| | 201 GSETPGTSES ATPESDKKYS IGLAIGTNSV GWAVITDEYK VPSKKFKVLG | |
| | 251 NTDRHSIKKN LIGALLFDSG ETAEATRLKR TARRRYTRRK NRICYLQEIF | |
| | 301 SNEMAKVDDS FFHRLEESFL VEEDKKHERH PIFGNIVDEV AYHEKYPTIY | |
| | 351 HLRKKLVDST DKADLRLIYL ALAHMIKFRG HFLIEGDLNP DNSDVDKLFI | |
| | 401 QLVQTYNQLF EENPINASGV DAKAILSARL SKSRRLENLI AQLPGEKKNG | |
| | 451 LFGNLIALSL GLTPNFKSNF DLAEDAKLQL SKDTYDDDLD NLLAQIGDQY | |
| | 501 ADLFLAAKNL SDAILLSDIL RVNTEITKAP LSASMIKRYD EHHQDLTLLK | |
| | 551 ALVRQQLPEK YKEIFFDQSK NGYAGYIDGG ASQEEFYKFI KPILEKMDGT | |
| | 601 EELLVKLNRE DLLRKQRTFD NGSIPHQIHL GELHAILRRQ EDFYPFLKDN | |
| | 651 REKIEKILTF RIPYYVGPLA RGNSRFAWMT RKSEETITPW NFEEVVDKGA | |
| | 701 SAQSFIERMT NFDKNLPNEK VLPKHSLLYE YFTVYNELTK VKYVTEGMRK | |
| | 751 PAFLSGEQKK AIVDLLFKTN RKVTVKQLKE DYFKKIECFD SVEISGVEDR | |
| | 801 FNASLGTYHD LLKIIKDKDF LDNEENEDIL EDIVLTLTLF EDREMIEERL | |
| | 851 KTYAHLFDDK VMKQLKRRRY TGWGRLSRKP INGIRDKQSG KTILDFLKSD | |
| | 901 GFANRNFMQL IHDDSLTFKE DIQKAQVSGQ GDSLHEHIAN LAGSPAIKKG | |
| | 951 ILQTVKVVDE LVKVMGRHKP ENIVIEMARE NQTTQKGQKN SRERMKRIEE | |
| | 1001 GIKELGSQIL KEHPVENTQL QNEKLYLYYL QNGRDMYVDQ ELDINRLSDY | |
| | 1051 DVDHIVPQSF LKDDSIDNKV LTRSDKNRGK SDNVPSEEVV KKMKNYWRQL | |
| | 1101 LNAKLITQRK FDNLTKAERG GLSELDKAGF IKRQLVETRQ ITKHVAQILD | |
| | 1151 SRMNTKYDEN DKLIREVKVI TLKSKLVSDF RKDFQFYKVR EINNYHHAHD | |
| | 1201 AYLNAVVGTA LIKKYPKLES EFVYGDYKVY DVRKMIAKSE QEIGKATAKY | |
| | 1251 FFYSNIMNFF KTEITLANGE IRKRPLIETN GETGEIVWDK GRDFATVRKV | |
| | 1301 LSMPQVNIVK KTEVQTGGFS KESILPKRNS DKLIARKKDW DPKKYGGFDS | |
| | 1351 PTVAYSVLVV AKVEKGKSKK LKSVKELLGI TIMERSSFEK NPIDFLEAKG | |
| | 1401 YKEVKKDLII KLPKYSLFEL ENGRKRMLAS AGELQKGNEL ALPSKYVNFL | |
| | 1451 YLASHYELKL GSPEDNEQKQ LFVEQHKHYL DEIIEQISEF SKRVILADAN | |
| | 1501 LDKVLSAYNK HRDKPIREQA ENIIHLFTLT NLGAPAAFKY FDTTIDRKRY | |
| | 1551 TSTKEVLDAT LIHQSITGLY ETRIDLSQLG GDSGGSTNLS DIIEKETGKQ | |
| | 1601 LVIQESILML PEEVEEVIGN KPESDILVHT AYDESTDENV MLLTSDAPEY | |
| | 1651 KPWALVIQDS NGENKIKMLS GGSPKKKRKV | |
| DNA construct | 1 Atatgccaag tacgccccct attgacgtca atgacggtaa atggcccgcc | 21 |
| | 51 tggcattatg cccagtacat gaccttatgg gactttccta cttggcagta | |
| | 101 catctacgta ttagtcatcg ctattaccat ggtgatgcgg ttttggcagt | |
| | 151 acatcaatgg gcgtggatag cggtttgact cacggggatt tccaagtctc | |
| | 201 caccccattg acgtcaatgg gagtttgttt tggcaccaaa atcaacggga | |
| | 251 ctttccaaaa tgtcgtaaca actccgcccc attgacgcaa atgggcggta | |
| | 301 ggcgtgtacg gtgggaggtc tatataagca gagctggttt agtgaaccgt | |
| | 351 cagatccgct agagatccgc ggccgctaat acgactcact atagggagag | |
| | 401 ccgccaccat ggaagccagc ccagcatccg ggcccagaca cttgatggat | |
| | 451 ccacacatat tcacttccaa ctttaacaat ggcattggaa ggcataagac | |
| | 501 ctacctgtgc tacgaagtgg agcgcctgga caatggcacc tcggtcaaga | |
| | 551 tggaccagca caggggcttt ctacacaacc aggctaagaa tcttctctgt | |
| | 601 ggcttttacg gccgccatgc ggagctgcgc ttcttggacc tggttccttc | |
| | 651 tttgcagttg gacccggccc agatctacag ggtcacttgg ttcatctcct | |
| | 701 ggagcccctg cttctcctgg ggctgtgccg gggaagtgcg tgcgttcctt | |
| | 751 caggagaaca cacacgtgag actgcgtatc ttcgctgccc gcatctatga | |
| | 801 ttacgacccc ctatataagg aggcactgca aatgctgcgg gatgctgggg | |
| | 851 cccaagtctc catcatgacc tacgatgaat ttaagcactg ctgggacacc | |
| | 901 tttgtggacc accagggatg tcccttccag ccctgggatg gactagatga | |
| | 951 gcacagccaa gccctgagtg ggaggctgcg ggccattctc cagaatcagg | |
| | 1001 gaaacagcgg cagcgagact cccgggacct cagagtccgc cacacccgaa | |
| | 1051 agtgataaaa agtattctat tggtttagcc atcggcacta attccgttgg | |
| | 1101 atgggctgtc ataaccgatg aatacaaagt accttcaaag aaatttaagg | |
| | 1151 tgttggggaa cacagaccgt cattcgatta aaaagaatct tatcggtgcc | |
| | 1201 ctcctattcg atagtggcga aacggcagag gcgactcgcc tgaaacgaac | |
| | 1251 cgctcggaga aggtatacac gtcgcaagaa ccgaatatgt tacttacaag | |
| | 1301 aaatttttag caatgagatg gccaaagttg acgattcttt ctttcaccgt | |
| | 1351 ttggaagagt ccttccttgt cgaagaggac aagaaacatg aacggcaccc | |
| | 1401 catctttgga aacatagtag atgaggtggc atatcatgaa aagtacccaa | |
| | 1451 cgatttatca cctcagaaaa aagctagttg actcaactga taaagcggac | |
| | 1501 ctgaggttaa tctacttggc tcttgcccat atgataaagt tccgtgggca | |
| | 1551 ctttctcatt gagggtgatc taaatccgga caactcggat gtcgacaaac | |
| | 1601 tgttcatcca gttagtacaa acctataatc agttgtttga agagaaccct | |
| | 1651 ataaatgcaa gtggcgtgga tgcgaaggct attcttagcg cccgcctctc | |
| | 1701 taaatcccga cggctagaaa acctgatcgc acaattaccc ggagagaaga | |
| | 1751 aaaatgggtt gttcggtaac cttatagcgc tctcactagg cctgacacca | |
| | 1801 aattttaagt cgaacttcga cttagctgaa gatgccaaat tgcagcttag | |
| | 1851 taaggacacg tacgatgacg atctcgacaa tctactggca caaattggag | |
| | 1901 atcagtatgc ggacttattt ttggctgcca aaaaccttag cgatgcaatc | |
| | 1951 ctcctatctg acatactgag agttaatact gagattacca aggcgccgtt | |
| | 2001 atccgcttca atgatcaaaa ggtacgatga acatcaccaa gacttgacac | |
| | 2051 ttctcaaggc cctagtccgt cagcaactgc ctgagaaata taaggaaata | |
| | 2101 ttctttgatc agtcgaaaaa cgggtacgca ggttatattg acggcggagc | |
| | 2151 gagtcaagag gaattctaca agtttatcaa acccatatta gagaagatgg | |
| | 2201 atgggacgga agagttgctt gtaaaactca atcgcgaaga tctactgcga | |
| | 2251 aagcagcgga ctttcgacaa cggtagcatt ccacatcaaa tccacttagg | |
| | 2301 cgaattgcat gctatactta gaaggcagga ggatttttat ccgttcctca | |
| | 2351 aagacaatcg tgaaaagatt gagaaaatcc taacctttcg cataccttac | |
| | 2401 tatgtgggac ccctggcccg agggaactct cggttcgcat ggatgacaag | |
| | 2451 aaagtccgaa gaaacgatta ctccatggaa ttttgaggaa gttgtcgata | |
| | 2501 aaggtgcgtc agctcaatcg ttcatcgaga ggatgaccaa ctttgaccag | |
| | 2551 aatttaccga acgaaaaagt attgcctaag cacagtttac tttacgagta | |
| | 2601 tttcacagtg tacaatgaac tcacgaaagt taagtatgtc actgagggca | |
| | 2651 tgcgtaaacc cgcctttcta agcggagaac agaagaaagc aatagtagat | |
| | 2701 ctgttattca agaccaaccg caaagtgaca gttaagcaat tgaaagagga | |
| | 2751 ctactttaag aaaattgaat gcttcgattc tgtcgagatc tccggggtag | |
| | 2801 aagatcgatt taatgcgtca cttggtacgt atcatgacct cctaaagata | |
| | 2851 attaaagata aggacttcct ggataacgaa gagaatgaag atatcttaga | |
| | 2901 agatatagtg ttgactctta ccctctttga agatcgggaa atgattgagg | |
| | 2951 aaagactaaa aacatacgct cacctgttcg acgataaggt tatgaaacag | |
| | 3001 ttaaagaggc gtcgctatac gggctgggga cgattgtcgc ggaaacttat | |
| | 3051 caacgggata agagacaagc aaagtggtaa aactattctc gattttctaa | |
| | 3101 agagcgacgg cttcgccaat aggaacttta tgcagctgat ccatgatgac | |
| | 3151 tctttaacct tcaaagagga tatacaaaag gcacaggttt ccggacaagg | |
| | 3201 ggactcattg cacgaacata ttgcgaatct tgctggttcg ccagccatca | |
| | 3251 aaaagggcat actccagaca gtcaaagtag tggatgagct agttaaggtc | |
| | 3301 atgggacgtc acaaaccgga aaacattgta atcgagatgg cacgcgaaaa | |
| | 3351 tcaaacgact cagaaggggc aaaaaaacag tcgagagcgg atgaagagaa | |
| | 3401 tagaagaggg tattaaagaa ctgggcagcc agatcttaaa ggagcatcct | |
| | 3451 gtggaaaata cccaattgca gaacgagaaa ctttacctct attacctaca | |
| | 3501 aaatggaagg gacatgtatg ttgatcagga actggacata aaccgtttat | |
| | 3551 ctgattacga cgtcgatcac attgtacccc aatccttttt gaaggacgat | |
| | 3601 tcaatcgaca ataaagtgct tacacgctcg gataagaacc gagggaaaag | |
| | 3651 tgacaatgtt ccaagcgagg aagtcgtaaa gaaaatgaag aactattggc | |
| | 3701 ggcagctcct aaatgcgaaa ctgataacgc aaagaaagtt cgataactta | |
| | 3751 actaaagctg agaggggtgg cttgtctgaa cttgacaagg ccggatttat | |
| | 3801 taaacgtcag ctcgtggaaa cccgccaaat cacaaagcat gttgcacaga | |
| | 3851 tactagattc ccgaatgaat acgaaatacg acgagaacga taagctgatt | |
| | 3901 cgggaagtca aagtaatcac tttaaagtca aaattggtgt cggacttcag | |
| | 3951 aaaggatttt caattctata aagttaggga gataaataac taccaccatg | |
| | 4001 cgcacgacgc ttatcttaat gccgtcgtag ggaccgcact cattaagaaa | |
| | 4051 tacccgaagc tagaaagtga gtttgtgtat ggtgattaca aagtttatga | |
| | 4101 cgtccgtaag atgatcgcga aaagcgaaca ggagataggc aaggctacag | |
| | 4151 ccaaatactt cttttattct aacattatga atttctttaa gacggaaatc | |
| | 4201 actctggcaa acggagagat acgcaaacga cctttaattg aaaccaatgg | |
| | 4251 ggagacaggt gaaatcgtat gggataaggg ccgggacttc gcgacggtga | |
| | 4301 gaaaagtttt gtccatgccc caagtcaaca tagtaaagaa aactgaggtg | |
| | 4351 cagaccggag ggttttcaaa ggaatcgatt cttccaaaaa ggaatagtga | |
| | 4401 taagctcatc gctcgtaaaa aggactggga cccgaaaaag tacggtggct | |
| | 4451 tcgatagccc tacagttgcc tattctgtcc tagtagtggc aaaagttgag | |
| | 4501 aagggaaaat ccaagaaact gaagtcagtc aaagaattat tggggataac | |
| | 4551 gattatggag cgctcgtctt ttgaaaagaa ccccatcgac ttccttgagg | |
| | 4601 cgaaaggtta caaggaagta aaaaaggatc tcataattaa actaccaaag | |
| | 4651 tatagtctgt ttgagttaga aaatggccga aaacggatgt tggctagcgc | |
| | 4701 cggagagctt caaaagggga acgaactcgc actaccgtct aaatacgtga | |
| | 4751 atttcctgta tttagcgtcc cattacgaga agttgaaagg ttcacctgaa | |
| | 4801 gataacgaac agaagcaact ttttgttgag cagcacaaac attatctcga | |
| | 4851 cgaaatcata gagcaaattt cggaattcag taagagagtc atcctagctg | |
| | 4901 atgccaatct ggacaaagta ttaagcgcat acaacaagca cagggataaa | |
| | 4951 cccatacgtg agcaggcgga aaatattatc catttgttta ctcttaccaa | |
| | 5001 cctcggcgct ccagccgcat tcaagtattt tgacacaacg gattcaccaa | |
| | 5051 aacgatacac ttctaccaag gaggtgctag acgcgacact gattcaccaa | |
| | 5101 tccatcacgg gattatatga aactcggata gatttgtcac agcttggggg | |
| | 5151 tgactctggt ggttctacta atctgtcaga tattattgaa aaggagaccg | |
| | 5201 gtaagcaact ggttatccag gaatccatcc tcatgctccc agaggaggtg | |
| | 5251 gaagaagtca ttgggaacaa gccggaaagc gatatactcg tgcacaccgc | |
| | 5301 ctacgacgag agcaccgacg agaatgtcat gcttctgact agcgacgccc | |
| | 5351 ctgaatacaa gccttgggct ctggtcatac aggatagcaa cggtgagaac | |
| | 5401 aagattaaga tgctctctgg tggttctccc aagaagaaga ggaaagtcta | |
| | 5451 accggtcatc atcaccatca ccattgagtt taaacccgct gatcagcctc | |
| | 5501 gactgtgcct tctagttgcc agccatctgt tgtttgcccc tcccccgtgc | |
| | 5551 cttccttgac cctggaaggt gccactccca ctgtcctttc ctaataaaat | |
| | 5601 gaggaaattg catcgcattg tctgagtagg tgtcattcta ttctgggggg | |
| | 5651 tggggtgggg caggacagca agggggagga ttgggaagac aatagcaggc | |
| | 5701 atgctgggga tgcggtgggc tctatggctt ctgaggcgga aagaaccagc | |
| | 5751 tggggctcga taccgtcgac ctctagctag agcttggcgt aatcatggtc | |
| | 5801 atagctgttt cctgtgtgaa attgttatcc gctcacaatt ccacacaaca | |
| | 5851 tacgagccgg aagcataaag tgtaaagcct agggtgccta atgagtgagc | |
| | 5901 taactcacat taattgcgtt gcgctcactg cccgctttcc agtcgggaaa | |
| | 5951 cctgtcgtgc cagctgcatt aatgaatcgg ccaacgcgcg gggagaggcg | |
| | 6001 gtttgcgtat tgggcgctct tccgcttcct cgctcactga ctcgctgcgc | |
| | 6051 tcggtcgttc ggctgcggcg agcggtatca gctcactcaa aggcggtaat | |
| | 6101 acggttatcc acagaatcag gggataacgc aggaaagaac atgtgagcaa | |
| | 6151 aaggccagca aaaggccagg aaccgtaaaa aggccgcgtt gctggcgttt | |
| | 6201 ttccataggc tccgcccccc tgacgagcat cacaaaaatc gacgctcaag | |
| | 6251 tcagaggtgg cgaaacccga caggactata aagataccag gcgtttcccc | |
| | 6301 ctggaagctc cctcgtgcgc tctcctgttc cgaccctgcc gcttaccgga | |
| | 6351 tacctgtccg cctttctccc ttcgggaagc gtggcgcttt ctcatagctc | |
| | 6401 acgctgtagg tatctcagtt cggtgtaggt cgttcgctcc aagctgggct | |
| | 6451 gtgtgcacga accccccgtt cagcccgacc gctgcgcctt atccggtaac | |
| | 6501 tatcgtcttg agtccaaccc ggtaagacac gacttatcgc cactggcagc | |
| | 6551 agccactggt aacaggatta gcagagcgag gtatgtaggc ggtgctacag | |
| | 6601 agttcttgaa gtggtggcct aactacggct acactagaag aacagtattt | |
| | 6651 ggtatctgcg ctctgctgaa gccagttacc ttcggaaaaa gagttggtag | |
| | 6701 ctcttgatcc ggcaaacaaa ccaccgctgg tagcggtggt ttttttgttt | |
| | 6751 gcaagcagca gattacgcgc agaaaaaaag gatctcaaga agatcctttg | |
| | 6801 atcttttcta cggggtctga cgctcagtgg aacgaaaact cacgttaagg | |
| | 6851 gattttggtc atgagattat caaaaaggat cttcacctag atccttttaa | |
| | 6901 attaaaaatg aagttttaaa tcaatctaaa gtatatatga gtaaacttgg | |
| | 6951 tctgacagtt accaatgctt aatcagtgag gcacctatct cagcgatctg | |
| | 7001 tctatttcgt tcatccatag ttgcctgact ccccgtcgtg tagataacta | |
| | 7051 cgatacggga gggcttacca tctggcccca gtgctgcaat gataccgcga | |
| | 7101 gacccacgct caccggctcc agatttatca gcaataaacc agccagccgg | |
| | 7151 aagggccgag cgcagaagtg gtcctgcaac tttatccgcc tccatccagt | |
| | 7201 ctattaattg ttgccgggaa gctagagtaa gtagttcgcc agttaatagt | |
| | 7251 ttgcgcaacg ttgttgccat tgctacaggc atcgtggtgt cacgctcgtc | |
| | 7301 gtttggtatg gcttcattca gctccggttc ccaacgatca aggcgagtta | |
| | 7351 catgatcccc catgttgtgc aaaaaagcgg ttagctcctt cggtcctccg | |
| | 7401 atcgttgtca gaagtaagtt ggccgcagtg ttatcactca tggttatggc | |
| | 7451 agcactgcat aattctctta ctgtcatgcc atccgtaaga tgcttttctg | |
| | 7501 tgactggtga gtactcaacc aagtcattct gagaatagtg tatgcggcga | |
| | 7551 ccgagttgct cttgcccggc gtcaatacgg gataataccg cgccacatag | |
| | 7601 cagaacttta aaagtgctca tcattggaaa acgttcttcg gggcgaaaac | |
| | 7651 tctcaaggat cttaccgctg ttgagatcca gttcgatgta acccactcgt | |
| | 7701 gcacccaact gatcttcagc atcttttact ttcaccagcg tttctgggtg | |
| | 7751 agcaaaaaca ggaaggcaaa atgccgcaaa aaagggaata agggcgacac | |
| | 7801 ggaaatgttg aatactcata ctcttccttt ttcaatatta ttgaagcatt | |
| | 7851 tatcagggtt attgtctcat gagcggatac atatttgaat gtatttagaa | |
| | 7901 aaataaacaa ataggggttc cgcgcacatt tccccgaaaa gtgccacctg | |
| | 7951 acgtcgacgg atcgggagat cgatctcccg atcccctagg gtcgactctc | |
| | 8001 agtacaatct gctctgatgc cgcatagtta agccagtatc tgctccctgc | |
| | 8051 ttgtgtgttg gaggtcgctg agtagtgcgc gagcaaaatt taagctacaa | |
| | 8101 caaggcaagg cttgaccgac aattgcatga agaatctgct tagggttagg | |
| | 8151 cgttttgcgc tgcttcgcga tgtacgggcc agatatacgc gttgacattg | |
| | 8201 attattgact agttattaat agtaatcaat tacggggtca ttagttcata | |
| | 8251 gcccatatat ggagttccgc gttacataac ttacggtaaa tggcccgcct | |
| | 8301 ggctgaccgc ccaacgaccc ccgcccattg acgtcaataa tgacgtatgt | |
| | 8351 tcccatagta acgccaatag ggactttcca ttgacgtcaa tgggtggagt | |
| | 8401 atttacggta aactgcccac ttggcagtac atcaagtgta tc | |
| Lb-dCas12a | 1 MSKLEKFTNC YSLSKTLRFK AIPVGKTQEN IDNKRLLVED EKRAEDYKGV | 37 |
| | 51 KKLLDRYYLS FINDVLHSIK LKNLNNYISL FRKKTRTEKE NKELENLEIN | |
| | 101 LRKEIAKAFK GNEGYKSLFK KDIIETILPE FLDDKDEIAL VNSFNGFTTA | |
| | 151 FTGFFDNREN MFSEEAKSTS IAFRCINENL TRYISNMDIF EKVDAIFDKH | |
| | 201 EVQEIKEKIL NSDYDVEDFF EGEFFNFVLT QEGIDVYNAI IGGFVTESGE | |
| | 251 KIKGLNEYIN LYNQKTKQKL PKFKPLYKQV LSDRESLSFY GEGYTSDEEV | |
| | 301 LEVFRNTLNK NSEIFSSIKK LEKLFKNFDE YSSAGIFVKN GPAISTISKD | |
| | 351 IFGEWNVIRD KWNAEYDDIH LKKKAVVTEK YEDDRRKSFK KIGSFSLEQL | |
| | 401 QEYADADLSV VEKLKEIIIQ KVDEIYKVYG SSEKLFDADF VLEKSLKKND | |
| | 451 AVVAIMKDLL DSVKSFENYI KAFFGEGKET NRDESFYGDF VLAYDILLKV | |
| | 501 DHIYDAIRNY VTQKPYSKDK FKLYFQNPQF MGGWDKDKET DYRATILRYG | |
| | 551 SKYYLAIMDK KYAKCLQKID KDDVNGNYEK INYKLLPGPN KMLPKVFFSK | |
| | 601 KWMAYYNPSE DIQKIYKNGT FKKGDMFNLN DCHKLIDFFK DSISRYPKWS | |
| | 651 NAYDFNFSET EKYKDIAGFY REVEEQGYKV SFESASKKEV DKLVEEGKLY | |
| | 701 MFQIYNKDFS DKSHGTPNLH TMYFKLLFDE NNHGQIRLSG GAELFMRRAS | |
| | 751 LKKEELVVHP ANSPIANKNP DNPKKTTTLS YDVYKDKRFS EDQYELHIPI | |
| | 801 AINKCPKNIF KINTEVRVLL KHDDNPYVIG IARGERNLLY IVVVDGKGNI | |
| | 851 VEQYSLNEII NNFNGIRIKT DYHSLLDKKE KERFEARQNW TSIENIKELK | |
| | 901 AGYISQVVHK ICELVEKYDA VIALADLNSG FKNSRVKVEK QVYQKFEKML | |
| | 951 IDKLNYMVDK KSNPCATGGA LKGYQITNKF ESFKSMSTQN GFIFYIPAWL | |
| | 1001 TSKIDPSTGF VNLLKTKYTS IADSKKFISS FDRIMYVPEE DLFEFALDYK | |
| | 1051 NFSRTDADYI KKWKLYSYGN RIRIFRNPKK NNVFDWEEVC LTSAYKELFN | |
| | 1101 KYGINYQQGD IRALLCEQSD KAFYSSFMAL MSLMLQMRNS ITGRTDVAFL | |
| | 1151 ISPVKNSDGI FYDSRNYEAQ ENAILPKNAD ANGAYNIARK VLWAIGQFKK | |
| | 1201 AEDEKLDKVK IAISNKEWLE YAQTSVKHGS | |
| AsCas12a | 1 MTQFEGFTNL YQVSKTLRFE LIPQGKTLKH IQEQGFIEED KARNDHYKEL | 38 |
| | 51 KPIIDRIYKT YADQCLQLVQ LDWENLSAAI DSYRKEKTEE TRNALIEEQA | |
| | 101 TYRNAIHDYF IGRTDNLTDA INKRHAEIYK GLFKAELFNG KVLKQLGTVT | |
| | 151 TTEHENALLR SFDKFTTYFS GFYENRKNVF SAEDISTAIP HRIVQDNFPK | |
| | 201 FKENCHIFTR LITAVPSLRE HFENVKKAIG IFVSTSIEEV FSFPFYNQLL | |
| | 251 TQTQIDLYNQ LLGGISREAG TEKIKGLNEV LNLAIQKNDE TAHIIASLPH | |
| | 301 RFIPLFKQIL SDRNTLSFIL EEFKSDEEVI QSFCKYKTLL RNENVLETAE | |
| | 351 ALFNELNSID LTHIFISHKK LETISSALCD HWDTLRNALY ERRISELTGK | |
| | 401 ITKSAKEKVQ RSLKHEDINL QEIISAAGKE LSEAFKQKTS EILSHAHAAL | |
| | 451 DQPLPTTLKK QEEKEILKSQ LDSLLGLYHL LDWFAVDESN EVDPEFSARL | |
| | 501 TGIKLEMEPS LSFYNKARNY ATKKPYSVEK FKLNFQMPTL ASGWDVNKEK | |
| | 551 NNGAILFVKN GLYYLGIMPK QKGRYKALSF EPTEKTSEGF DKMYYDYFPD | |
| | 601 AAKMIPKCST QLKAVTAHFQ THTTPILLSN NFIEPLEITK EIYDLNNPEK | |
| | 651 EPKKFQTAYA KKTGDQKGYR EALCKWIDFT RDFLSKYTKT TSIDLSSLRP | |
| | 701 SSQYKDLGEY YAELNPLLYH ISFQRIAEKE IMDAVETGKL YLFQIYNKDF | |
| | 751 AKGHHGKPNL HTLYWTGLFS PENLAKTSIK LNGQAELFYR PKSRMKRMAH | |
| | 801 RLGEKMLNKK LKDQKTPIPD TLYQELYDYV NHRLSHDLSD EARALLPNVI | |
| | 851 TKEVSHEIIK DRRFTSDKFF FHVPITLNYQ AANSPSKFNQ RVNAYLKEHP | |
| | 901 ETPIIGIDRG ERNLIYITVI DSTGKILEQR SLNTIQQFDY QKKLDNREKE | |
| | 951 RVAARQAWSV VGTIKDLKQG YLSQVIHEIV DLMIHYQAVV VLENLNFGFK | |
| | 1001 SKRTGIAEKA VYQQFEKMLI DKLNCLVLKD YPAEKVGGVL NPYQLTDQFT | |
| | 1051 SFAKMGTQSG FLFYVPAPYT SKIDPLTGFV DPFVWKTIKN HESRKHFLEG | |
| | 1101 FDFLHYDVKT GDFILHFKMN RNLSFQRGLP GFMPAWDIVF EKNETQFDAK | |
| | 1151 GTPFIAGKRI VPVIENHRFT GRYRDLYPAN ELIALLEEKG IVFRDGSNIL | |
| | 1201 PKLLENDDSH AIDTMVALIR SVLQMRNSNA ATGEDYINSP VRDLNGVCFD | |
| | 1251 SRFQNPEWPM DADANGAYHI ALKGQLLLNH LKESKDLKLQ NGISNQDWLA | |
| | 1301 YIQELRN | |
| FnCas12a | 1 MSIYQEFVNK YSLSKTLRFE LIPQGKTLEN IKARGLILDD EKRAKDYKKA | 39 |
| | 51 KQIIDKYHQF FIEEILSSVC ISEDLLQNYS DVYFKLKKSD DDNLQKDFKS | |
| | 101 AKDTIKKQIS EYIKDSEKFK NLFNQNLIDA KKGQESDLIL WLKQSKDNGI | |
| | 151 ELFKANSDIT DIDEALEIIK SFKGWTTYFK GFHENRKNVY SSNDIPTSII | |
| | 201 YRIVDDNLPK FLENKAKYES LKDKAPEAIN YEQIKKDLAE ELTFDIDYKT | |
| | 251 SEVNQRVFSL DEVFEIANFN NYLNQSGITK FNTIIGGKFV NGENTKRKGI | |
| | 301 NEYINLYSQQ INDKTLKKYK MSVLFKQILS DTESKSFVID KLEDDSDVVT | |
| | 351 TMQSFYEQIA AFKTVEEKSI KETLSLLFDD LKAQKLDLSK IYFKNDKSLT | |
| | 401 DLSQQVFDDY SVIGTAVLEY ITQQIAPKNL DNPSKKEQEL IAKKTEKAKY | |
| | 451 LSLETIKLAL EEFNKHRDID KQCRFEEILA NFAAIPMIFD EIAQNKDNLA | |
| | 501 QISIKYQNQG KKDLLQASAE DDVKAIKDLL DQTNNLLHKL KIFHISQSED | |
| | 551 KANILDKDEH FYLVFEECYF ELANIVPLYN KIRNYITQKP YSDEKFKLNF | |
| | 601 ENSTLANGWD KNKEPDNTAI LFIKDDKYYL GVMNKKNNKI FDDKAIKENK | |
| | 651 GEGYKKIVYK LLPGANKMLP KVFFSAKSIK FYNPSEDILR IRNHSTHTKN | |
| | 701 GSPQKGYEKF EFNIEDCRKF IDFYKQSISK HPEWKDFGFR FSDTQRYNSI | |
| | 751 DEFYREVENQ GYKLTFENIS ESYIDSVVNQ GKLYLFQIYN KDFSAYSKGR | |
| | 801 PNLHTLYWKA LFDERNLQDV VYKLNGEAEL FYRKQSIPKK ITHPAKEAIA | |
| | 851 NKNKDNPKKE SVFEYDLIKD KRFTEDKFFF HCPITINFKS SGANKFNDEI | |
| | 901 NLLLKEKAND VHILSIDRGE RHLAYYTLVD GKGNIIKQDT FNIIGNDRMK | |
| | 951 TNYHDKLAAI EKDRDSARKD WKKINNIKEM KEGYLSQVVH EIAKLVIEYN | |
| | 1001 AIVVFEDLNF GFKRGRFKVE KQVYQKLEKM LIEKLNYLVF KDNEFDKTGG | |
| | 1051 VLRAYQLTAP FETFKKMGKQ TGIIYYVPAG FTSKICPVTG FVNQLYPKYE | |
| | 1101 SVSKSQEFFS KFDKICYNLD KGYFEFSFDY KNFGDKAAKG KWTIASFGSR | |
| | 1151 LINFRNSDKN HNWDTREVYP TKELEKLLKD YSIEYGHGEC IKAAICGESD | |
| | 1201 KKFFAKLTSV LNTILQMRNS KTGTELDYLI SPVADVNGNF FDSRQAPKNM | |
| | 1251 PQDADANGAY HIGLKGLMLL GRIKNNQEGK KLNLVIKNEE YFEFVQNRNN | |
| dCas12a-hA3A-BE | 1 MPKKKRKVME ASPASGPRHL MDPHIFTSNF NNGIGRHKTY LCYEVERLDN | 40 |
| | 51 GTSVKMDQHR GFLHNQAKNL LCGFYGRHAE LRFLDLVPSL QLDPAQIYRV | |
| | 101 TWFISWSPCF SWGCAGEVRA FLQENTHVRL RIFAARIYDY DPLYKEALQM | |
| | 151 LRDAGAQVSI MTYDEFKHCW DTFVDHQGCP FQPWDGLDEH SQALSGRLRA | |
| | 201 ILQNQGNSGS ETPGTSESAT PESMSKLEKF TNCYSLSKTL RFKAIPVGKT | |
| | 251 QENIDNKRLL VEDEKRAEDY KGVKKLLDRY YLSFINDVLH SIKLKNLNNY | |
| | 301 ISLFRKKTRT EKENKELENL EINLRKEIAK AFKGNEGYKS LFKKDIIETI | |
| | 351 LPEFLDDKDE IALVNSFNGF TTAFTGFFDN RENMFSEEAK STSIAFRCIN | |
| | 401 ENLTRYISNM DIFEKVDAIF DKHEVQEIKE KILNSDYDVE DFFEGEFFNF | |
| | 451 VLTQEGIDVY NAIIGGFVTE SGEKIKGLNE YINLYNQKTK QKLPKFKPLY | |
| | 501 KQVLSDRESL SFYGEGYTSD EEVLEVFRNT LNKNSEIFSS IKKLEKLFKN | |
| | 551 FDEYSSAGIF VKNGPAISTI SKDIFGEWNV IRDKWNAEYD DIHLKKKAVV | |
| | 601 TEKYEDDRRK SFKKIGSFSL EQLQEYADAD LSVVEKLKEI IIQKVDEIYK | |
| | 651 VYGSSEKLFD ADFVLEKSLK KNDAVVAIMK DLLDSVKSFE NYIKAFFGEG | |
| | 701 KETNRDESFY GDFVLAYDIL LKVDHIYDAI RNYVTQKPYS KDKFKLYFQN | |
| | 751 PQFMGGWDKD KETDYRATIL RYGSKYYLAI MDKKYAKCLQ KIDKDDVNGN | |
| | 801 YEKINYKLLP GPNKMLPKVF FSKKWMAYYN PSEDIQKIYK NGTFKKGDMF | |
| | 851 NLNDCHKLID FFKDSISRYP KWSNAYDFNF SETEKYKDIA GFYREVEEQG | |
| | 901 YKVSFESASK KEVDKLVEEG KLYMFQIYNK DFSDKSHGTP NLHTMYFKLL | |
| | 951 FDENNHGQIR LSGGAELFMR RASLKKEELV VHPANSPIAN KNPDNPKKTT | |
| | 1001 TLSYDVYKDK RFSEDQYELH IPIAINKCPK NIFKINTEVR VLLKHDDNPY | |
| | 1051 VIGIARGERN LLYIVVVDGK GNIVEQYSLN EIINNFNGIR IKTDYHSLLD | |
| | 1101 KKEKERFEAR QNWTSIENIK ELKAGYISQV VHKICELVEK YDAVIALADL | |
| | 1151 NSGFKNSRVK VEKQVYQKFE KMLIDKLNYM VDKKSNPCAT GGALKGYQIT | |
| | 1201 NKFESFKSMS TQNGFIFYIP AWLTSKIDPS TGFVNLLKTK YTSIADSKKF | |
| | 1251 ISSFDRIMYV PEEDLFEFAL DYKNFSRTDA DYIKKWKLYS YGNRIRIFRN | |
| | 1301 PKKNNVFDWE EVCLTSAYKE LFNKYGINYQ QGDIRALLCE QSDKAFYSSF | |
| | 1351 MALMSLMLQM RNSITGRTDV AFLISPVKNS DGIFYDSRNY EAQENAILPK | |
| | 1401 NADANGAYNI ARKVLWAIGQ FKKAEDEKLD KVKIAISNKE WLEYAQTSVK | |
| | 1451 HGSPKKKRKV SGGSTNLSDI IEKETGKQLV IQESILMLPE EVEEVIGNKP | |
| | 1501 ESDILVHTAY DESTDENVML LTSDAPEYKP WALVIQDSNG ENKIKMLSGG | |
| | 1551 SPKKKRKV | |
| dCas12a-hA3A- | 1 MPKKKRKVME ASPASGPRHL MDPHIFTSNF NNGIGRHKTY LCYEVERLDN | 41 |
| BE-W98Y | 51 GTSVKMDQHR GFLHNQAKNL LCGFYGRHAE LRFLDLVPSL QLDPAQIYRV | |
| | 101 TWFISYSPCF SWGCAGEVRA FLQENTHVRL RIFAARIYDY DPLYKRALQM | |
| | 151 LRDAGAQVSI MTYDEFKHCW DTFVDHQGCP FQPWDGLDEH SQALSGRLRA | |
| | 201 ILQNQGNSGS ETPGTSESAT PESMSKLEKF TNCYSLSKTL RFKAIPVGKT | |
| | 251 QENIDNKRLL VEDEKRAEDY KGVKKLLDRY YLSFINDVLH SIKLKNLNNY | |
| | 301 ISLFRKKTRT EKENKELENL EINLRKEIAK AFKGNEGYKS LFKKDIIETI | |
| | 351 LPEFLDDKDE IALVNSFNGF TTAFTGFFDN RENMFSEEAK STSIAFRCIN | |
| | 401 ENLTRYISNM DIFEKVDAIF DKHEVQEIKE KILNSDYDVE DFFEGEFFNF | |
| | 451 VLTQEGIDVY NAIIGGFVTE SGEKIKGLNE YINLYNQKTK QKLPKFKPLY | |
| | 501 KQVLSDRESL SFYGEGYTSD EEVLEVFRNT LNKNSEIFSS IKKLEKLFKN | |
| | 551 FDEYSSAGIF VKNGPAISTI SKDIFGEWNV IRDKWNAEYD DIHLKKKAVV | |
| | 601 TEKYEDDRRK SFKKIGSFSL EQLQEYADAD LSVVEKLKEI IIQKVDEIYK | |
| | 651 VYGSSEKLFD ADFVLEKSLK KNDAVVAIMK DLLDSVKSFE NYIKAFFGEG | |
| | 701 KETNRDESFY GDFVLAYDIL LKVDHIYDAI RNYVTQKPYS KDKFKLYFQN | |
| | 751 PQFMGGWDKD KETDYRATIL RYGSKYYLAI MDKKYAKCLQ KIDKDDVNGN | |
| | 801 YEKINYKLLP GPNKMLPKVF FSKKWMAYYN PSEDIQKIYK NGTFKKGDMF | |
| | 851 NLNDCHKLID FFKDSISRYP KWSNAYDFNF SETEKYKDIA GFYREVEEQG | |
| | 901 YKVSFESASK KEVDKLVEEG KLYMFQIYNK DFSDKSHGTP NLHTMYFKLL | |
| | 951 FDENNHGQIR LSGGAELFMR RASLKKEELV VHPANSPIAN KNPDNPKKTT | |
| | 1001 TLSYDVYKDK RFSEDQYELH IPIAINKCPK NIFKINTEVR VLLKHDDNPY | |
| | 1051 VIGIARGERN LLYIVVVDGK GNIVEQYSLN EIINNFNGIR IKTDYHSLLD | |
| | 1101 KKEKERFEAR QNWTSIENIK ELKAGYISQV VHKICELVEK YDAVIALADL | |
| | 1151 NSGFKNSRVK VEKQVYQKFE KMLIDKLNYM VDKKSNPCAT GGALKGYQIT | |
| | 1201 NKFESFKSMS TQNGFIFYIP AWLTSKIDPS TGFVNLLKTK YTSIADSKKF | |
| | 1251 ISSFDRIMYV PEEDLFEFAL DYKNFSRTDA DYIKKWKLYS YGNRIRIFRN | |
| | 1301 PKKNNVFDWE EVCLTSAYKE LFNKYGINYQ QGDIRALLCE QSDKAFYSSF | |
| | 1351 MALMSLMLQM RNSITGRTDV AFLISPVKNS DGIFYDSRNY EAQENAILPK | |
| | 1401 NADANGAYNI ARKVLWAIGQ FKKAEDEKLD KVKIAISNKE WLEYAQTSVK | |
| | 1451 HGSPKKKRKV SGGSTNLSDI IEKETGKQLV IQESILMLPE EVEEVIGNKP | |
| | 1501 ESDILVHTAY DESTDENVML LTSDAPEYKP WALVIQDSNG ENKIKMLSGG | |
| | 1551 SPKKKRKV | |
| dCas12a-hA3A- | 1 MPKKKRKVME ASPASGPRHL MDPHIFTSNF NNGIGRHKTY LCYEVERLDN | 42 |
| BE-W104A | 51 GTSVKMDQHR GFLHNQAKNL LCGFYGRHAE LRFLDLVPSL QLDPAQIYRV | |
| | 101 TWFISWSPCF SAGCAGEVRA FLQENTHVRL RIFAARIYDY DPLYKRALQM | |
| | 151 LRDAGAQVSI MTYDEFKHCW DTFVDHQGCP FQPWDGLDEH SQALSGRLRA | |
| | 201 ILQNQGNSGS ETPGTSESAT PESMSKLEKF TNCYSLSKTL RFKAIPVGKT | |
| | 251 QENIDNKRLL VEDEKRAEDY KGVKKLLDRY YLSFINDVLH SIKLKNLNNY | |
| | 301 ISLFRKKTRT EKENKELENL EINLRKEIAK AFKGNEGYKS LFKKDIIETI | |
| | 351 LPEFLDDKDE IALVNSFNGF TTAFTGFFDN RENMFSEEAK STSIAFRCIN | |
| | 401 ENLTRYISNM DIFEKVDAIF DKHEVQEIKE KILNSDYDVE DFFEGEFFNF | |
| | 451 VLTQEGIDVY NAIIGGFVTE SGEKIKGLNE YINLYNQKTK QKLPKFKPLY | |
| | 501 KQVLSDRESL SFYGEGYTSD EEVLEVFRNT LNKNSEIFSS IKKLEKLFKN | |
| | 551 FDEYSSAGIF VKNGPAISTI SKDIFGEWNV IRDKWNAEYD DIHLKKKAVV | |
| | 601 TEKYEDDRRK SFKKIGSFSL EQLQEYADAD LSVVEKLKEI IIQKVDEIYK | |
| | 651 VYGSSEKLFD ADFVLEKSLK KNDAVVAIMK DLLDSVKSFE NYIKAFFGEG | |
| | 701 KETNRDESFY GDFVLAYDIL LKVDHIYDAI RNYVTQKPYS KDKFKLYFQN | |
| | 751 PQFMGGWDKD KETDYRATIL RYGSKYYLAI MDKKYAKCLQ KIDKDDVNGN | |
| | 801 YEKINYKLLP GPNKMLPKVF FSKKWMAYYN PSEDIQKIYK NGTFKKGDMF | |
| | 851 NLNDCHKLID FFKDSISRYP KWSNAYDFNF SETEKYKDIA GFYREVEEQG | |
| | 901 YKVSFESASK KEVDKLVEEG KLYMFQIYNK DFSDKSHGTP NLHTMYFKLL | |
| | 951 FDENNHGQIR LSGGAELFMR RASLKKEELV VHPANSPIAN KNPDNPKKTT | |
| | 1001 TLSYDVYKDK RFSEDQYELH IPIAINKCPK NIFKINTEVR VLLKHDDNPY | |
| | 1051 VIGIARGERN LLYIVVVDGK GNIVEQYSLN EIINNFNGIR IKTDYHSLLD | |
| | 1101 KKEKERFEAR QNWTSIENIK ELKAGYISQV VHKICELVEK YDAVIALADL | |
| | 1151 NSGFKNSRVK VEKQVYQKFE KMLIDKLNYM VDKKSNPCAT GGALKGYQIT | |
| | 1201 NKFESFKSMS TQNGFIFYIP AWLTSKIDPS TGFVNLLKTK YTSIADSKKF | |
| | 1251 ISSFDRIMYV PEEDLFEFAL DYKNFSRTDA DYIKKWKLYS YGNRIRIFRN | |
| | 1301 PKKNNVFDWE EVCLTSAYKE LFNKYGINYQ QGDIRALLCE QSDKAFYSSF | |
| | 1351 MALMSLMLQM RNSITGRTDV AFLISPVKNS DGIFYDSRNY EAQENAILPK | |
| | 1401 NADANGAYNI ARKVLWAIGQ FKKAEDEKLD KVKIAISNKE WLEYAQTSVK | |
| | 1451 HGSPKKKRKV SGGSTNLSDI IEKETGKQLV IQESILMLPE EVEEVIGNKP | |
| | 1501 ESDILVHTAY DESTDENVML LTSDAPEYKP WALVIQDSNG ENKIKMLSGG | |
| | 1551 SPKKKRKV | |
| dCas12a-hA3A- | 1 MPKKKRKVME ASPASGPRHL MDPHIFTSNF NNGIGRHKTY LCYEVERLDN | 43 |
| BE-P134Y | 51 GTSVKMDQHR GFLHNQAKNL LCGFYGRHAE LRFLDLVPSL QLDPAQIYRV | |
| | 101 TWFISWSPCF SWGCAGEVRA FLQENTHVRL RIFAARIYDY DYLYKRALQM | |
| | 151 LRDAGAQVSI MTYDEFKHCW DTFVDHQGCP FQPWDGLDEH SQALSGRLRA | |
| | 201 ILQNQGNSGS ETPGTSESAT PESMSKLEKF TNCYSLSKTL RFKAIPVGKT | |
| | 251 QENIDNKRLL VEDEKRAEDY KGVKKLLDRY YLSFINDVLH SIKLKNLNNY | |
| | 301 ISLFRKKTRT EKENKELENL EINLRKEIAK AFKGNEGYKS LFKKDIIETI | |
| | 351 LPEFLDDKDE IALVNSFNGF TTAFTGFFDN RENMFSEEAK STSIAFRCIN | |
| | 401 ENLTRYISNM DIFEKVDAIF DKHEVQEIKE KILNSDYDVE DFFEGEFFNF | |
| | 451 VLTQEGIDVY NAIIGGFVTE SGEKIKGLNE YINLYNQKTK QKLPKFKPLY | |
| | 501 KQVLSDRESL SFYGEGYTSD EEVLEVFRNT LNKNSEIFSS IKKLEKLFKN | |
| | 551 FDEYSSAGIF VKNGPAISTI SKDIFGEWNV IRDKWNAEYD DIHLKKKAVV | |
| | 601 TEKYEDDRRK SFKKIGSFSL EQLQEYADAD LSVVEKLKEI IIQKVDEIYK | |
| | 651 VYGSSEKLFD ADFVLEKSLK KNDAVVAIMK DLLDSVKSFE NYIKAFFGEG | |
| | 701 KETNRDESFY GDFVLAYDIL LKVDHIYDAI RNYVTQKPYS KDKFKLYFQN | |
| | 751 PQFMGGWDKD KETDYRATIL RYGSKYYLAI MDKKYAKCLQ KIDKDDVNGN | |
| | 801 YEKINYKLLP GPNKMLPKVF FSKKWMAYYN PSEDIQKIYK NGTFKKGDMF | |
| | 851 NLNDCHKLID FFKDSISRYP KWSNAYDFNF SETEKYKDIA GFYREVEEQG | |
| | 901 YKVSFESASK KEVDKLVEEG KLYMFQIYNK DFSDKSHGTP NLHTMYFKLL | |
| | 951 FDENNHGQIR LSGGAELFMR RASLKKEELV VHPANSPIAN KNPDNPKKTT | |
| | 1001 TLSYDVYKDK RFSEDQYELH IPIAINKCPK NIFKINTEVR VLLKHDDNPY | |
| | 1051 VIGIARGERN LLYIVVVDGK GNIVEQYSLN EIINNFNGIR IKTDYHSLLD | |
| | 1101 KKEKERFEAR QNWTSIENIK ELKAGYISQV VHKICELVEK YDAVIALADL | |
| | 1151 NSGFKNSRVK VEKQVYQKFE KMLIDKLNYM VDKKSNPCAT GGALKGYQIT | |
| | 1201 NKFESFKSMS TQNGFIFYIP AWLTSKIDPS TGFVNLLKTK YTSIADSKKF | |
| | 1251 ISSFDRIMYV PEEDLFEFAL DYKNFSRTDA DYIKKWKLYS YGNRIRIFRN | |
| | 1301 PKKNNVFDWE EVCLTSAYKE LFNKYGINYQ QGDIRALLCE QSDKAFYSSF | |
| | 1351 MALMSLMLQM RNSITGRTDV AFLISPVKNS DGIFYDSRNY EAQENAILPK | |
| | 1401 NADANGAYNI ARKVLWAIGQ FKKAEDEKLD KVKIAISNKE WLEYAQTSVK | |
| | 1451 HGSPKKKRKV SGGSTNLSDI IEKETGKQLV IQESILMLPE EVEEVIGNKP | |
| | 1501 ESDILVHTAY DESTDENVML LTSDAPEYKP WALVIQDSNG ENKIKMLSGG | |
| | 1551 SPKKKRKV | |
| dCas12a-hA3A- | 1 MPKKKRKVME ASPASGPRHL MDPHIFTSNF NNGIGRHKTY LCYEVERLDN | 44 |
| BE-W98Y-W104A | 51 GTSVKMDQHR GFLHNQAKNL LCGFYGRHAE LRFLDLVPSL QLDPAQIYRV | |
| | 101 TWFISYSPCF SAGCAGEVRA FLQENTHVRL RIFAARIYDY DPLYKRALQM | |
| | 151 LRDAGAQVSI MTYDEFKHCW DTFVDHQGCP FQPWDGLDEH SQALSGRLRA | |
| | 201 ILQNQGNSGS ETPGTSESAT PESMSKLEKF TNCYSLSKTL RFKAIPVGKT | |
| | 251 QENIDNKRLL VEDEKRAEDY KGVKKLLDRY YLSFINDVLH SIKLKNLNNY | |
| | 301 ISLFRKKTRT EKENKELENL EINLRKEIAK AFKGNEGYKS LFKKDIIETI | |
| | 351 LPEFLDDKDE IALVNSFNGF TTAFTGFFDN RENMFSEEAK STSIAFRCIN | |
| | 401 ENLTRYISNM DIFEKVDAIF DKHEVQEIKE KILNSDYDVE DFFEGEFFNF | |
| | 451 VLTQEGIDVY NAIIGGFVTE SGEKIKGLNE YINLYNQKTK QKLPKFKPLY | |
| | 501 KQVLSDRESL SFYGEGYTSD EEVLEVFRNT LNKNSEIFSS IKKLEKLFKN | |
| | 551 FDEYSSAGIF VKNGPAISTI SKDIFGEWNV IRDKWNAEYD DIHLKKKAVV | |
| | 601 TEKYEDDRRK SFKKIGSFSL EQLQEYADAD LSVVEKLKEI IIQKVDEIYK | |
| | 651 VYGSSEKLFD ADFVLEKSLK KNDAVVAIMK DLLDSVKSFE NYIKAFFGEG | |
| | 701 KETNRDESFY GDFVLAYDIL LKVDHIYDAI RNYVTQKPYS KDKFKLYFQN | |
| | 751 PQFMGGWDKD KETDYRATIL RYGSKYYLAI MDKKYAKCLQ KIDKDDVNGN | |
| | 801 YEKINYKLLP GPNKMLPKVF FSKKWMAYYN PSEDIQKIYK NGTFKKGDMF | |
| | 851 NLNDCHKLID FFKDSISRYP KWSNAYDFNF SETEKYKDIA GFYREVEEQG | |
| | 901 YKVSFESASK KEVDKLVEEG KLYMFQIYNK DFSDKSHGTP NLHTMYFKLL | |
| | 951 FDENNHGQIR LSGGAELFMR RASLKKEELV VHPANSPIAN KNPDNPKKTT | |
| | 1001 TLSYDVYKDK RFSEDQYELH IPIAINKCPK NIFKINTEVR VLLKHDDNPY | |
| | 1051 VIGIARGERN LLYIVVVDGK GNIVEQYSLN EIINNFNGIR IKTDYHSLLD | |
| | 1101 KKEKERFEAR QNWTSIENIK ELKAGYISQV VHKICELVEK YDAVIALADL | |
| | 1151 NSGFKNSRVK VEKQVYQKFE KMLIDKLNYM VDKKSNPCAT GGALKGYQIT | |
| | 1201 NKFESFKSMS TQNGFIFYIP AWLTSKIDPS TGFVNLLKTK YTSIADSKKF | |
| | 1251 ISSFDRIMYV PEEDLFEFAL DYKNFSRTDA DYIKKWKLYS YGNRIRIFRN | |
| | 1301 PKKNNVFDWE EVCLTSAYKE LFNKYGINYQ QGDIRALLCE QSDKAFYSSF | |
| | 1351 MALMSLMLQM RNSITGRTDV AFLISPVKNS DGIFYDSRNY EAQENAILPK | |
| | 1401 NADANGAYNI ARKVLWAIGQ FKKAEDEKLD KVKIAISNKE WLEYAQTSVK | |
| | 1451 HGSPKKKRKV SGGSTNLSDI IEKETGKQLV IQESILMLPE EVEEVIGNKP | |
| | 1501 ESDILVHTAY DESTDENVML LTSDAPEYKP WALVIQDSNG ENKIKMLSGG | |
| | 1551 SPKKKRKV | |
| dCas12a-hA3A- | 1 MPKKKRKVME ASPASGPRHL MDPHIFTSNF NNGIGRHKTY LCYEVERLDN | 45 |
| BE-W98Y-P134Y | 51 GTSVKMDQHR GFLHNQAKNL LCGFYGRHAE LRFLDLVPSL QLDPAQIYRV | |
| | 101 TWFISYSPCF SWGCAGEVRA FLQENTHVRL RIFAARIYDY DYLYKRALQM | |
| | 151 LRDAGAQVSI MTYDEFKHCW DTFVDHQGCP FQPWDGLDEH SQALSGRLRA | |
| | 201 ILQNQGNSGS ETPGTSESAT PESMSKLEKF TNCYSLSKTL RFKAIPVGKT | |
| | 251 QENIDNKRLL VEDEKRAEDY KGVKKLLDRY YLSFINDVLH SIKLKNLNNY | |
| | 301 ISLFRKKTRT EKENKELENL EINLRKEIAK AFKGNEGYKS LFKKDIIETI | |
| | 351 LPEFLDDKDE IALVNSFNGF TTAFTGFFDN RENMFSEEAK STSIAFRCIN | |
| | 401 ENLTRYISNM DIFEKVDAIF DKHEVQEIKE KILNSDYDVE DFFEGEFFNF | |
| | 451 VLTQEGIDVY NAIIGGFVTE SGEKIKGLNE YINLYNQKTK QKLPKFKPLY | |
| | 501 KQVLSDRESL SFYGEGYTSD EEVLEVFRNT LNKNSEIFSS IKKLEKLFKN | |
| | 551 FDEYSSAGIF VKNGPAISTI SKDIFGEWNV IRDKWNAEYD DIHLKKKAVV | |
| | 601 TEKYEDDRRK SFKKIGSFSL EQLQEYADAD LSVVEKLKEI IIQKVDEIYK | |
| | 651 VYGSSEKLFD ADFVLEKSLK KNDAVVAIMK DLLDSVKSFE NYIKAFFGEG | |
| | 701 KETNRDESFY GDFVLAYDIL LKVDHIYDAI RNYVTQKPYS KDKFKLYFQN | |
| | 751 PQFMGGWDKD KETDYRATIL RYGSKYYLAI MDKKYAKCLQ KIDKDDVNGN | |
| | 801 YEKINYKLLP GPNKMLPKVF FSKKWMAYYN PSEDIQKIYK NGTFKKGDMF | |
| | 851 NLNDCHKLID FFKDSISRYP KWSNAYDFNF SETEKYKDIA GFYREVEEQG | |
| | 901 YKVSFESASK KEVDKLVEEG KLYMFQIYNK DFSDKSHGTP NLHTMYFKLL | |
| | 951 FDENNHGQIR LSGGAELFMR RASLKKEELV VHPANSPIAN KNPDNPKKTT | |
| | 1001 TLSYDVYKDK RFSEDQYELH IPIAINKCPK NIFKINTEVR VLLKHDDNPY | |
| | 1051 VIGIARGERN LLYIVVVDGK GNIVEQYSLN EIINNFNGIR IKTDYHSLLD | |
| | 1101 KKEKERFEAR QNWTSIENIK ELKAGYISQV VHKICELVEK YDAVIALADL | |
| | 1151 NSGFKNSRVK VEKQVYQKFE KMLIDKLNYM VDKKSNPCAT GGALKGYQIT | |
| | 1201 NKFESFKSMS TQNGFIFYIP AWLTSKIDPS TGFVNLLKTK YTSIADSKKF | |
| | 1251 ISSFDRIMYV PEEDLFEFAL DYKNFSRTDA DYIKKWKLYS YGNRIRIFRN | |
| | 1301 PKKNNVFDWE EVCLTSAYKE LFNKYGINYQ QGDIRALLCE QSDKAFYSSF | |
| | 1351 MALMSLMLQM RNSITGRTDV AFLISPVKNS DGIFYDSRNY EAQENAILPK | |
| | 1401 NADANGAYNI ARKVLWAIGQ FKKAEDEKLD KVKIAISNKE WLEYAQTSVK | |
| | 1451 HGSPKKKRKV SGGSTNLSDI IEKETGKQLV IQESILMLPE EVEEVIGNKP | |
| | 1501 ESDILVHTAY DESTDENVML LTSDAPEYKP WALVIQDSNG ENKIKMLSGG | |
| | 1551 SPKKKRKV | |
| dCas12a-hA3A- | 1 MPKKKRKVME ASPASGPRHL MDPHIFTSNF NNGIGRHKTY LCYEVERLDN | 46 |
| BE-W104A-P134Y | 51 GTSVKMDQHR GFLHNQAKNL LCGFYGRHAE LRFLDLVPSL QLDPAQIYRV | |
| | 101 TWFISWSPCF SAGCAGEVRA FLQENTHVRL RIFAARIYDY DYLYKRALQM | |
| | 151 LRDAGAQVSI MTYDEFKHCW DTFVDHQGCP FQPWDGLDEH SQALSGRLRA | |
| | 201 ILQNQGNSGS ETPGTSESAT PESMSKLEKF TNCYSLSKTL RFKAIPVGKT | |
| | 251 QENIDNKRLL VEDEKRAEDY KGVKKLLDRY YLSFINDVLH SIKLKNLNNY | |
| | 301 ISLFRKKTRT EKENKELENL EINLRKEIAK AFKGNEGYKS LFKKDIIETI | |
| | 351 LPEFLDDKDE IALVNSFNGF TTAFTGFFDN RENMFSEEAK STSIAFRCIN | |
| | 401 ENLTRYISNM DIFEKVDAIF DKHEVQEIKE KILNSDYDVE DFFEGEFFNF | |
| | 451 VLTQEGIDVY NAIIGGFVTE SGEKIKGLNE YINLYNQKTK QKLPKFKPLY | |
| | 501 KQVLSDRESL SFYGEGYTSD EEVLEVFRNT LNKNSEIFSS IKKLEKLFKN | |
| | 551 FDEYSSAGIF VKNGPAISTI SKDIFGEWNV IRDKWNAEYD DIHLKKKAVV | |
| | 601 TEKYEDDRRK SFKKIGSFSL EQLQEYADAD LSVVEKLKEI IIQKVDEIYK | |
| | 651 VYGSSEKLFD ADFVLEKSLK KNDAVVAIMK DLLDSVKSFE NYIKAFFGEG | |
| | 701 KETNRDESFY GDFVLAYDIL LKVDHIYDAI RNYVTQKPYS KDKFKLYFQN | |
| | 751 PQFMGGWDKD KETDYRATIL RYGSKYYLAI MDKKYAKCLQ KIDKDDVNGN | |
| | 801 YEKINYKLLP GPNKMLPKVF FSKKWMAYYN PSEDIQKIYK NGTFKKGDMF | |
| | 851 NLNDCHKLID FFKDSISRYP KWSNAYDFNF SETEKYKDIA GFYREVEEQG | |
| | 901 YKVSFESASK KEVDKLVEEG KLYMFQIYNK DFSDKSHGTP NLHTMYFKLL | |
| | 951 FDENNHGQIR LSGGAELFMR RASLKKEELV VHPANSPIAN KNPDNPKKTT | |
| | 1001 TLSYDVYKDK RFSEDQYELH IPIAINKCPK NIFKINTEVR VLLKHDDNPY | |
| | 1051 VIGIARGERN LLYIVVVDGK GNIVEQYSLN EIINNFNGIR IKTDYHSLLD | |
| | 1101 KKEKERFEAR QNWTSIENIK ELKAGYISQV VHKICELVEK YDAVIALADL | |
| | 1151 NSGFKNSRVK VEKQVYQKFE KMLIDKLNYM VDKKSNPCAT GGALKGYQIT | |
| | 1201 NKFESFKSMS TQNGFIFYIP AWLTSKIDPS TGFVNLLKTK YTSIADSKKF | |
| | 1251 ISSFDRIMYV PEEDLFEFAL DYKNFSRTDA DYIKKWKLYS YGNRIRIFRN | |
| | 1301 PKKNNVFDWE EVCLTSAYKE LFNKYGINYQ QGDIRALLCE QSDKAFYSSF | |
| | 1351 MALMSLMLQM RNSITGRTDV AFLISPVKNS DGIFYDSRNY EAQENAILPK | |
| | 1401 NADANGAYNI ARKVLWAIGQ FKKAEDEKLD KVKIAISNKE WLEYAQTSVK | |
| | 1451 HGSPKKKRKV SGGSTNLSDI IEKETGKQLV IQESILMLPE EVEEVIGNKP | |
| | 1501 ESDILVHTAY DESTDENVML LTSDAPEYKP WALVIQDSNG ENKIKMLSGG | |
| | 1551 SPKKKRKV | |
| dCas12a-hA3A- | 1 MPKKKRKVME ASPASGPRHL MDPHIFTSNF NNGIGRHKTY LCYEVERLDN | 47 |
| BE-W98Y-W104A- | 51 GTSVKMDQHR GFLHNQAKNL LCGFYGRHAE LRFLDLVPSL QLDPAQIYRV | |
| Y130F | 101 TWFISYSPCF SAGCAGEVRA FLQENTHVRL RIFAARIFDY DPLYKRALQM | |
| | 151 LRDAGAQVSI MTYDEFKHCW DTFVDHQGCP FQPWDGLDEH SQALSGRLRA | |
| | 201 ILQNQGNSGS ETPGTSESAT PESMSKLEKF TNCYSLSKTL RFKAIPVGKT | |
| | 251 QENIDNKRLL VEDEKRAEDY KGVKKLLDRY YLSFINDVLH SIKLKNLNNY | |
| | 301 ISLFRKKTRT EKENKELENL EINLRKEIAK AFKGNEGYKS LFKKDIIETI | |
| | 351 LPEFLDDKDE IALVNSFNGF TTAFTGFFDN RENMFSEEAK STSIAFRCIN | |
| | 401 ENLTRYISNM DIFEKVDAIF DKHEVQEIKE KILNSDYDVE DFFEGEFFNF | |
| | 451 VLTQEGIDVY NAIIGGFVTE SGEKIKGLNE YINLYNQKTK QKLPKFKPLY | |
| | 501 KQVLSDRESL SFYGEGYTSD EEVLEVFRNT LNKNSEIFSS IKKLEKLFKN | |
| | 551 FDEYSSAGIF VKNGPAISTI SKDIFGEWNV IRDKWNAEYD DIHLKKKAVV | |
| | 601 TEKYEDDRRK SFKKIGSFSL EQLQEYADAD LSVVEKLKEI IIQKVDEIYK | |
| | 651 VYGSSEKLFD ADFVLEKSLK KNDAVVAIMK DLLDSVKSFE NYIKAFFGEG | |
| | 701 KETNRDESFY GDFVLAYDIL LKVDHIYDAI RNYVTQKPYS KDKFKLYFQN | |
| | 751 PQFMGGWDKD KETDYRATIL RYGSKYYLAI MDKKYAKCLQ KIDKDDVNGN | |
| | 801 YEKINYKLLP GPNKMLPKVF FSKKWMAYYN PSEDIQKIYK NGTFKKGDMF | |
| | 851 NLNDCHKLID FFKDSISRYP KWSNAYDFNF SETEKYKDIA GFYREVEEQG | |
| | 901 YKVSFESASK KEVDKLVEEG KLYMFQIYNK DFSDKSHGTP NLHTMYFKLL | |
| | 951 FDENNHGQIR LSGGAELFMR RASLKKEELV VHPANSPIAN KNPDNPKKTT | |
| | 1001 TLSYDVYKDK RFSEDQYELH IPIAINKCPK NIFKINTEVR VLLKHDDNPY | |
| | 1051 VIGIARGERN LLYIVVVDGK GNIVEQYSLN EIINNFNGIR IKTDYHSLLD | |
| | 1101 KKEKERFEAR QNWTSIENIK ELKAGYISQV VHKICELVEK YDAVIALADL | |
| | 1151 NSGFKNSRVK VEKQVYQKFE KMLIDKLNYM VDKKSNPCAT GGALKGYQIT | |
| | 1201 NKFESFKSMS TQNGFIFYIP AWLTSKIDPS TGFVNLLKTK YTSIADSKKF | |
| | 1251 ISSFDRIMYV PEEDLFEFAL DYKNFSRTDA DYIKKWKLYS YGNRIRIFRN | |
| | 1301 PKKNNVFDWE EVCLTSAYKE LFNKYGINYQ QGDIRALLCE QSDKAFYSSF | |
| | 1351 MALMSLMLQM RNSITGRTDV AFLISPVKNS DGIFYDSRNY EAQENAILPK | |
| | 1401 NADANGAYNI ARKVLWAIGQ FKKAEDEKLD KVKIAISNKE WLEYAQTSVK | |
| | 1451 HGSPKKKRKV SGGSTNLSDI IEKETGKQLV IQESILMLPE EVEEVIGNKP | |
| | 1501 ESDILVHTAY DESTDENVML LTSDAPEYKP WALVIQDSNG ENKIKMLSGG | |
| | 1551 SPKKKRKV | |
| dCas12a-hA3A-BE- | 1 MPKKKRKVME ASPASGPRHL MDPHIFTSNF NNGIGRHKTY LCYEVERLDN | 48 |
| W98Y-W104A-Y132D | 51 GTSVKMDQHR GFLHNQAKNL LCGFYGRHAE LRFLDLVPSL QLDPAQIYRV | |
| | 101 TWFISYSPCF SAGCAGEVRA FLQENTHVRL RIFAARIYDD DPLYKRALQM | |
| | 151 LRDAGAQVSI MTYDEFKHCW DTFVDHQGCP FQPWDGLDEH SQALSGRLRA | |
| | 201 ILQNQGNSGS ETPGTSESAT PESMSKLEKF TNCYSLSKTL RFKAIPVGKT | |
| | 251 QENIDNKRLL VEDEKRAEDY KGVKKLLDRY YLSFINDVLH SIKLKNLNNY | |
| | 301 ISLFRKKTRT EKENKELENL EINLRKEIAK AFKGNEGYKS LFKKDIIETI | |
| | 351 LPEFLDDKDE IALVNSFNGF TTAFTGFFDN RENMFSEEAK STSIAFRCIN | |
| | 401 ENLTRYISNM DIFEKVDAIF DKHEVQEIKE KILNSDYDVE DFFEGEFFNF | |
| | 451 VLTQEGIDVY NAIIGGFVTE SGEKIKGLNE YINLYNQKTK QKLPKFKPLY | |
| | 501 KQVLSDRESL SFYGEGYTSD EEVLEVFRNT LNKNSEIFSS IKKLEKLFKN | |
| | 551 FDEYSSAGIF VKNGPAISTI SKDIFGEWNV IRDKWNAEYD DIHLKKKAVV | |
| | 601 TEKYEDDRRK SFKKIGSFSL EQLQEYADAD LSVVEKLKEI IIQKVDEIYK | |
| | 651 VYGSSEKLFD ADFVLEKSLK KNDAVVAIMK DLLDSVKSFE NYIKAFFGEG | |
| | 701 KETNRDESFY GDFVLAYDIL LKVDHIYDAI RNYVTQKPYS KDKFKLYFQN | |
| | 751 PQFMGGWDKD KETDYRATIL RYGSKYYLAI MDKKYAKCLQ KIDKDDVNGN | |
| | 801 YEKINYKLLP GPNKMLPKVF FSKKWMAYYN PSEDIQKIYK NGTFKKGDMF | |
| | 851 NLNDCHKLID FFKDSISRYP KWSNAYDFNF SETEKYKDIA GFYREVEEQG | |
| | 901 YKVSFESASK KEVDKLVEEG KLYMFQIYNK DFSDKSHGTP NLHTMYFKLL | |
| | 951 FDENNHGQIR LSGGAELFMR RASLKKEELV VHPANSPIAN KNPDNPKKTT | |
| | 1001 TLSYDVYKDK RFSEDQYELH IPIAINKCPK NIFKINTEVR VLLKHDDNPY | |
| | 1051 VIGIARGERN LLYIVVVDGK GNIVEQYSLN EIINNFNGIR IKTDYHSLLD | |
| | 1101 KKEKERFEAR QNWTSIENIK ELKAGYISQV VHKICELVEK YDAVIALADL | |
| | 1151 NSGFKNSRVK VEKQVYQKFE KMLIDKLNYM VDKKSNPCAT GGALKGYQIT | |
| | 1201 NKFESFKSMS TQNGFIFYIP AWLTSKIDPS TGFVNLLKTK YTSIADSKKF | |
| | 1251 ISSFDRIMYV PEEDLFEFAL DYKNFSRTDA DYIKKWKLYS YGNRIRIFRN | |
| | 1301 PKKNNVFDWE EVCLTSAYKE LFNKYGINYQ QGDIRALLCE QSDKAFYSSF | |
| | 1351 MALMSLMLQM RNSITGRTDV AFLISPVKNS DGIFYDSRNY EAQENAILPK | |
| | 1401 NADANGAYNI ARKVLWAIGQ FKKAEDEKLD KVKIAISNKE WLEYAQTSVK | |
| | 1451 HGSPKKKRKV SGGSTNLSDI IEKETGKQLV IQESILMLPE EVEEVIGNKP | |
| | 1501 ESDILVHTAY DESTDENVML LTSDAPEYKP WALVIQDSNG ENKIKMLSGG | |
| | 1551 SPKKKRKV | |
| dCas12a-hA3A-BE- | 1 MPKKKRKVME ASPASGPRHL MDPHIFTSNF NNGIGRHKTY LCYEVERLDN | 49 |
| W104A-Y130F-P134Y | 51 GTSVKMDQHR GFLHNQAKNL LCGFYGRHAE LRFLDLVPSL QLDPAQIYRV | |
| | 101 TWFISWSPCF SAGCAGEVRA FLQENTHVRL RIFAARIFDY DYLYKRALQM | |
| | 151 LRDAGAQVSI MTYDEFKHCW DTFVDHQGCP FQPWDGLDEH SQALSGRLRA | |
| | 201 ILQNQGNSGS ETPGTSESAT PESMSKLEKF TNCYSLSKTL RFKAIPVGKT | |
| | 251 QENIDNKRLL VEDEKRAEDY KGVKKLLDRY YLSFINDVLH SIKLKNLNNY | |
| | 301 ISLFRKKTRT EKENKELENL EINLRKEIAK AFKGNEGYKS LFKKDIIETI | |
| | 351 LPEFLDDKDE IALVNSFNGF TTAFTGFFDN RENMFSEEAK STSIAFRCIN | |
| | 401 ENLTRYISNM DIFEKVDAIF DKHEVQEIKE KILNSDYDVE DFFEGEFFNF | |
| | 451 VLTQEGIDVY NAIIGGFVTE SGEKIKGLNE YINLYNQKTK QKLPKFKPLY | |
| | 501 KQVLSDRESL SFYGEGYTSD EEVLEVFRNT LNKNSEIFSS IKKLEKLFKN | |
| | 551 FDEYSSAGIF VKNGPAISTI SKDIFGEWNV IRDKWNAEYD DIHLKKKAVV | |
| | 601 TEKYEDDRRK SFKKIGSFSL EQLQEYADAD LSVVEKLKEI IIQKVDEIYK | |
| | 651 VYGSSEKLFD ADFVLEKSLK KNDAVVAIMK DLLDSVKSFE NYIKAFFGEG | |
| | 701 KETNRDESFY GDFVLAYDIL LKVDHIYDAI RNYVTQKPYS KDKFKLYFQN | |
| | 751 PQFMGGWDKD KETDYRATIL RYGSKYYLAI MDKKYAKCLQ KIDKDDVNGN | |
| | 801 YEKINYKLLP GPNKMLPKVF FSKKWMAYYN PSEDIQKIYK NGTFKKGDMF | |
| | 851 NLNDCHKLID FFKDSISRYP KWSNAYDFNF SETEKYKDIA GFYREVEEQG | |
| | 901 YKVSFESASK KEVDKLVEEG KLYMFQIYNK DFSDKSHGTP NLHTMYFKLL | |
| | 951 FDENNHGQIR LSGGAELFMR RASLKKEELV VHPANSPIAN KNPDNPKKTT | |
| | 1001 TLSYDVYKDK RFSEDQYELH IPIAINKCPK NIFKINTEVR VLLKHDDNPY | |
| | 1051 VIGIARGERN LLYIVVVDGK GNIVEQYSLN EIINNFNGIR IKTDYHSLLD | |
| | 1101 KKEKERFEAR QNWTSIENIK ELKAGYISQV VHKICELVEK YDAVIALADL | |
| | 1151 NSGFKNSRVK VEKQVYQKFE KMLIDKLNYM VDKKSNPCAT GGALKGYQIT | |
| | 1201 NKFESFKSMS TQNGFIFYIP AWLTSKIDPS TGFVNLLKTK YTSIADSKKF | |
| | 1251 ISSFDRIMYV PEEDLFEFAL DYKNFSRTDA DYIKKWKLYS YGNRIRIFRN | |
| | 1301 PKKNNVFDWE EVCLTSAYKE LFNKYGINYQ QGDIRALLCE QSDKAFYSSF | |
| | 1351 MALMSLMLQM RNSITGRTDV AFLISPVKNS DGIFYDSRNY EAQENAILPK | |
| | 1401 NADANGAYNI ARKVLWAIGQ FKKAEDEKLD KVKIAISNKE WLEYAQTSVK | |
| | 1451 HGSPKKKRKV SGGSTNLSDI IEKETGKQLV IQESILMLPE EVEEVIGNKP | |
| | 1501 ESDILVHTAY DESTDENVML LTSDAPEYKP WALVIQDSNG ENKIKMLSGG | |
| | 1551 SPKKKRKV | |
| dCas12a-hA3A- | 1 MPKKKRKVME ASPASGPRHL MDPHIFTSNF NNGIGRHKTY LCYEVERLDN | 50 |
| BE-W104A-Y132D- | 51 GTSVKMDQHR GFLHNQAKNL LCGFYGRHAE LRFLDLVPSL QLDPAQIYRV | |
| P134Y | 101 TWFISWSPCF SAGCAGEVRA FLQENTHVRL RIFAARIYDD DYLYKRALQM | |
| | 151 LRDAGAQVSI MTYDEFKHCW DTFVDHQGCP FQPWDGLDEH SQALSGRLRA | |
| | 201 ILQNQGNSGS ETPGTSESAT PESMSKLEKF TNCYSLSKTL RFKAIPVGKT | |
| | 251 QENIDNKRLL VEDEKRAEDY KGVKKLLDRY YLSFINDVLH SIKLKNLNNY | |
| | 301 ISLFRKKTRT EKENKELENL EINLRKEIAK AFKGNEGYKS LFKKDIIETI | |
| | 351 LPEFLDDKDE IALVNSFNGF TTAFTGFFDN RENMFSEEAK STSIAFRCIN | |
| | 401 ENLTRYISNM DIFEKVDAIF DKHEVQEIKE KILNSDYDVE DFFEGEFFNF | |
| | 451 VLTQEGIDVY NAIIGGFVTE SGEKIKGLNE YINLYNQKTK QKLPKFKPLY | |
| | 501 KQVLSDRESL SFYGEGYTSD EEVLEVFRNT LNKNSEIFSS IKKLEKLFKN | |
| | 551 FDEYSSAGIF VKNGPAISTI SKDIFGEWNV IRDKWNAEYD DIHLKKKAVV | |
| | 601 TEKYEDDRRK SFKKIGSFSL EQLQEYADAD LSVVEKLKEI IIQKVDEIYK | |
| | 651 VYGSSEKLFD ADFVLEKSLK KNDAVVAIMK DLLDSVKSFE NYIKAFFGEG | |
| | 701 KETNRDESFY GDFVLAYDIL LKVDHIYDAI RNYVTQKPYS KDKFKLYFQN | |
| | 751 PQFMGGWDKD KETDYRATIL RYGSKYYLAI MDKKYAKCLQ KIDKDDVNGN | |
| | 801 YEKINYKLLP GPNKMLPKVF FSKKWMAYYN PSEDIQKIYK NGTFKKGDMF | |
| | 851 NLNDCHKLID FFKDSISRYP KWSNAYDFNF SETEKYKDIA GFYREVEEQG | |
| | 901 YKVSFESASK KEVDKLVEEG KLYMFQIYNK DFSDKSHGTP NLHTMYFKLL | |
| | 951 FDENNHGQIR LSGGAELFMR RASLKKEELV VHPANSPIAN KNPDNPKKTT | |
| | 1001 TLSYDVYKDK RFSEDQYELH IPIAINKCPK NIFKINTEVR VLLKHDDNPY | |
| | 1051 VIGIARGERN LLYIVVVDGK GNIVEQYSLN EIINNFNGIR IKTDYHSLLD | |
| | 1101 KKEKERFEAR QNWTSIENIK ELKAGYISQV VHKICELVEK YDAVIALADL | |
| | 1151 NSGFKNSRVK VEKQVYQKFE KMLIDKLNYM VDKKSNPCAT GGALKGYQIT | |
| | 1201 NKFESFKSMS TQNGFIFYIP AWLTSKIDPS TGFVNLLKTK YTSIADSKKF | |
| | 1251 ISSFDRIMYV PEEDLFEFAL DYKNFSRTDA DYIKKWKLYS YGNRIRIFRN | |
| | 1301 PKKNNVFDWE EVCLTSAYKE LFNKYGINYQ QGDIRALLCE QSDKAFYSSF | |
| | 1351 MALMSLMLQM RNSITGRTDV AFLISPVKNS DGIFYDSRNY EAQENAILPK | |
| | 1401 NADANGAYNI ARKVLWAIGQ FKKAEDEKLD KVKIAISNKE WLEYAQTSVK | |
| | 1451 HGSPKKKRKV SGGSTNLSDI IEKETGKQLV IQESILMLPE EVEEVIGNKP | |
| | 1501 ESDILVHTAY DESTDENVML LTSDAPEYKP WALVIQDSNG ENKIKMLSGG | |
| | 1551 SPKKKRKV | |

The present disclosure also provides isolated polynucleotides or nucleic acid molecules (e.g., SEQ ID NO:21) encoding the fusion proteins, variants or derivatives thereof of the disclosure. Methods of making fusion proteins are well known in the art and described herein.

### Compositions and Methods

The present disclosure also provides compositions and methods. Such compositions comprise an effective amount of a fusion protein, and an acceptable carrier. In some embodiments, the composition further includes a guide RNA that has a desired complementarity to a target DNA. Such a composition can be used for base editing in a sample.

The fusion proteins and the compositions can be used for base editing. In one embodiment, a method for editing a target polynucleotide is provided, comprising contacting to the target polynucleotide a fusion protein of the present disclosure and a guide RNA having at least partial sequence complementarity to the target polynucleotide, wherein the editing comprises deamination of a cytosine (C) in the target polynucleotide.

It is shown that the presently disclosed fusion proteins can edit cytosine at any location and in any context, such as in CpC, ApC, GpC, TpC, CpA, CpG, CpC, CpT. It is surprising and unexpected, however, that these fusion proteins can edit C in a GpC dinucleotide context, and even when the C is methylated.

The contacting between the fusion protein (and the guide RNA) and the target polynucleotide can be in vitro, in particular in a cell culture. When the contacting is ex vivo, or in vivo, the fusion proteins can exhibit clinical/therapeutic significance.

### EXAMPLES

### Example 1: Base Editors

Human apolipoprotein B mRNA editing enzyme catalytic subunit 3A (APOBEC3A, hA3A; SEQ ID NO:1) was included in an expression vector that further included a Cas9 nickase (SEQ ID NO:11) and a uracil-DNA-glycosylase inhibitor [Bacillus phage AR9] (SEQ ID NO:12). The Cas9 nickase contained a Asp10Ala mutation that inactivated its double strand nuclease activity, while allowing it to introduce a nick on one of the strands.

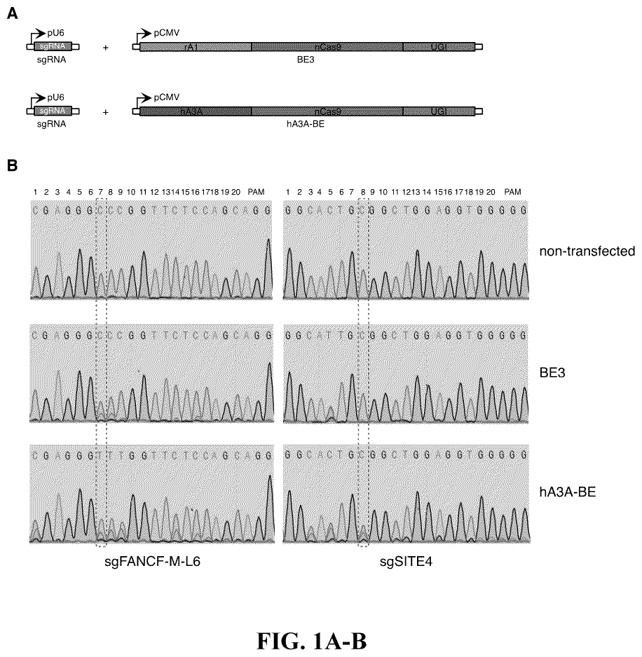

The fusion vector, hA3A-nCas9-UGI (hA3A-BE, SEQ ID NO:21), and a sgRNA expression vector were co-transfected into eukaryotic cells (FIG. 1A) to perform C-to-T base editing at sgRNA target site in the genome. After PCR amplification of the target genomic DNA, the C-to-T base editing efficiency at targeted site in genome were determined through Sanger DNA sequencing. As illustrated in two sgRNA target sites (sgFANCF-M-L6 and sgSITE4), efficient C-to-T base editing was executed on C of GpC through co-expressing hA3A-BE and sgRNA, as compared to co-expressing BE3 (APOBEC1-nCas9-UGI) and sgRNA (FIG. 1B, dashed box).

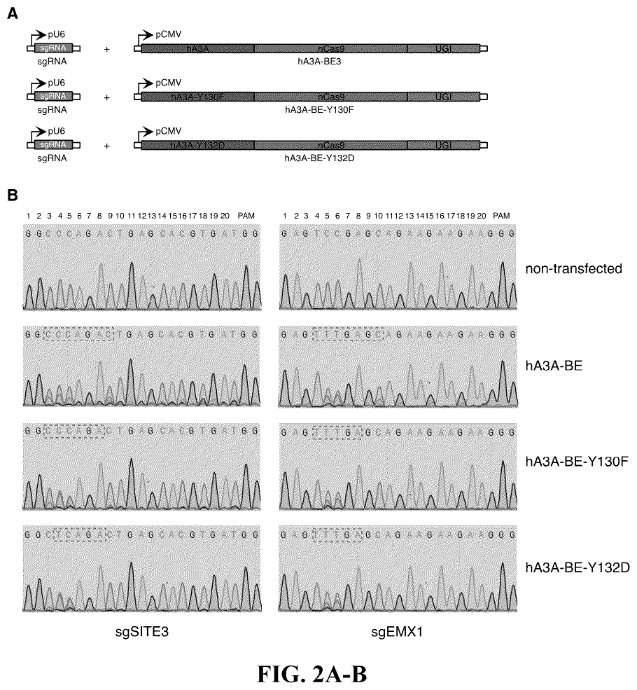

Next, mutations Y130F (SEQ ID NO:2) and Y132D (SEQ ID NO:3) were individually introduced into the hA3A gene in the construct, thereby generating the base editor hA3A-BE-Y130F or hA3A-BE-Y132D (FIG. 2A). The Y130F and Y132D mutations in hA3A-BE narrowed the window of base editing, and further improved the editing precision of hA3A-BE (FIG. 2B).

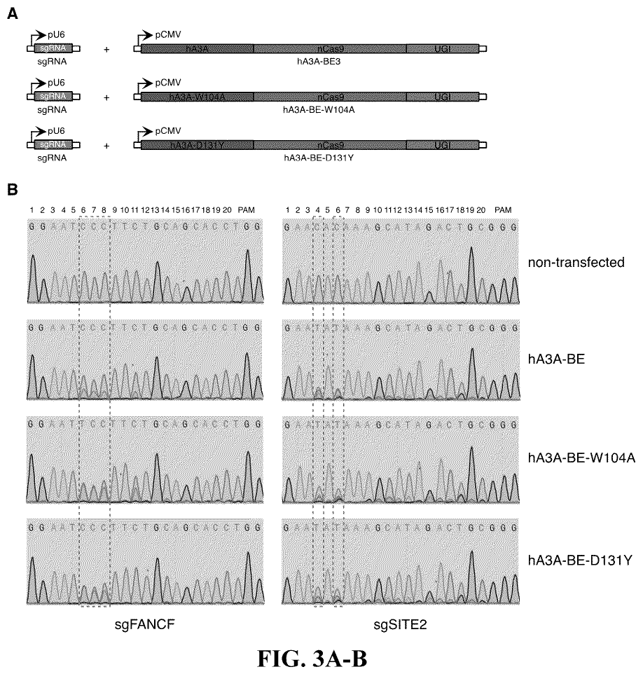

Furthermore, the mutations W104A (SEQ ID NO:4) and D131Y (SEQ ID NO:5) were individually introduced into the hA3A gene of hA3A-BE, thereby generating the base editor hA3A-BE-W104A or hA3A-BE-D131Y (FIG. 3A). Both hA3A-BE-W104A and hA3A-BE-D131Y increased the efficiency of desired C to T base substitutions (FIG. 3B), achieving even higher efficiency of base editing as compared to hA3A-BE.

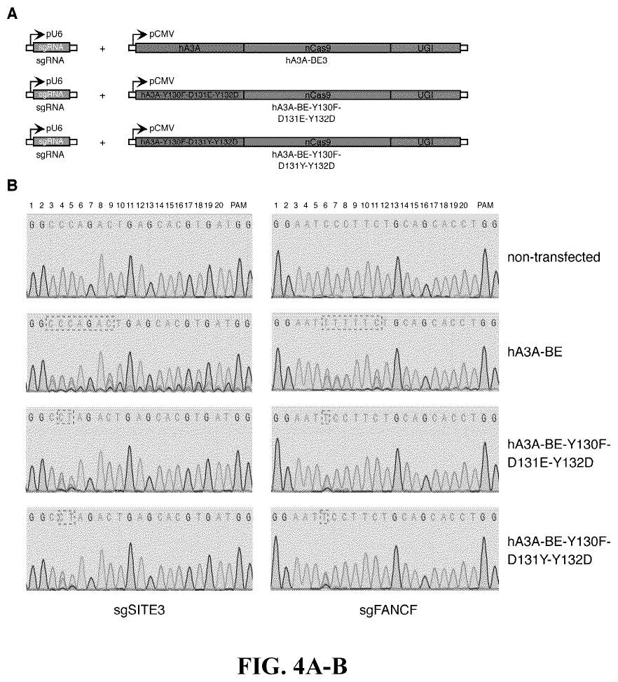

In a further experiment, three amino acid changes (Y130E-D131E-Y132D, SEQ ID NO:22 or Y130E-D131Y-Y132D, SEQ ID NO:23) of human APOBEC3A (hA3A) in hA3A-BE3 (FIG. 4A) were tested and it was found that these two base editors (hA3A-BE-Y130E-D131E-Y132D and hA3A-BE-Y130E-D131Y-Y132D) have more narrowed editing windows (position 4-6 in target region) and therefore higher editing precision (FIG. 4B).

### Example 2: Efficient Base Editing in Methylated Regions with a Human APOBEC3A-Cas9 Fusion

Base editors (BEs) enable the generation of targeted single-nucleotide mutations, but currently used rat APOBEC1-based BEs are relatively inefficient in editing cytosines in highly-methylated regions or in GpC contexts. By screening a variety of APOBEC/AID deaminases, this example shows that human APOBEC3A-conjugated BEs and versions engineered to have narrower editing windows can mediate efficient C-to-T base editing in regions with high methylation levels and GpC dinucleotide content.

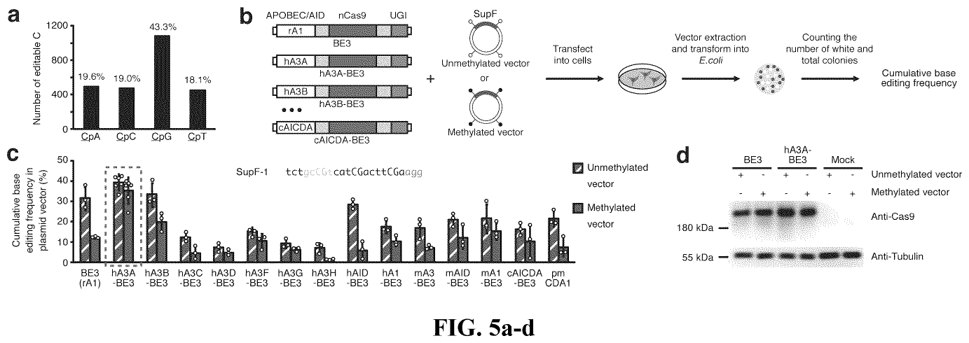

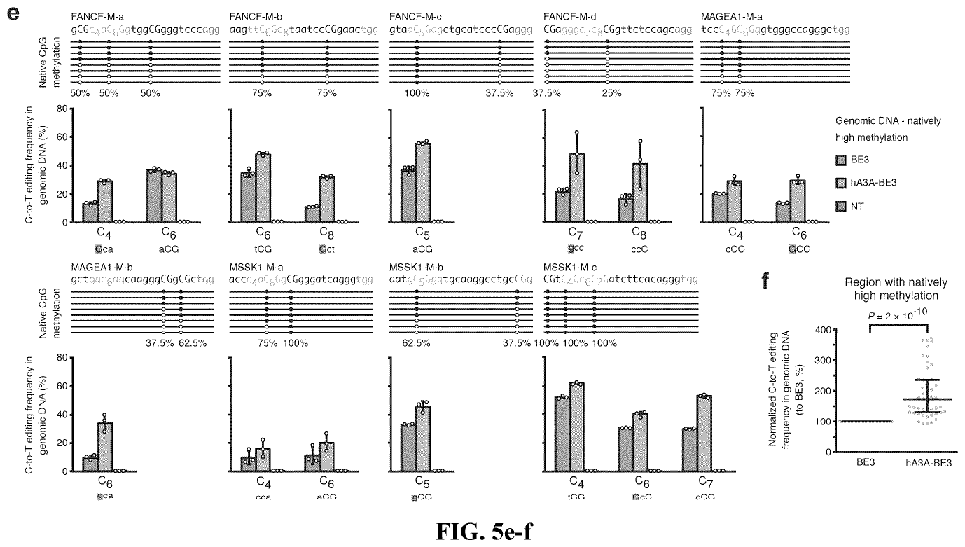

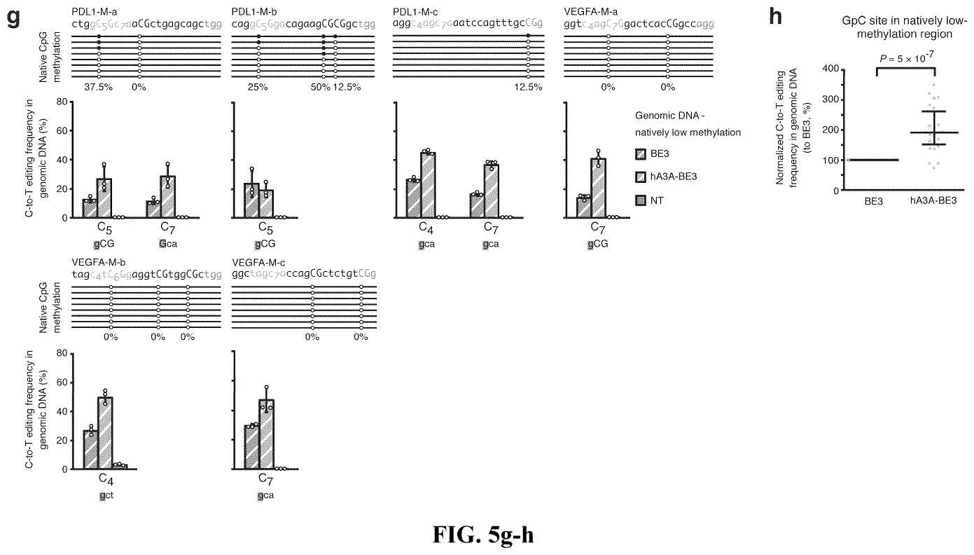

Base editors (BEs), which combine a cytidine deaminase with Cas9 or Cpf1, have been successfully applied to perform targeted base editing, including C-to-T. Numerous human diseases have been reported to be driven by point mutations in genomic DNAs. With recently developed BEs, these disease-related point mutations can be potentially corrected, providing new therapeutic options. By analyzing disease-related T-to-C mutations that can be theoretically reverted to thymines by BEs, the example found that ˜43% of them are on cytosines in the context of CpG dinucleotides (FIG. 5a). It is well known that C of CpG is usually methylated in mammalian cells, and methylation of C strongly suppresses cytidine deamination catalyzed by some APOBEC/AID deaminases. This example shows that CpG dinucleotide methylation hinders the C-to-T base editing by current BEs and has successfully developed BEs for efficient C-to-T base editing in highly methylated regions.

### Methods and Materials

### Plasmid Construction

Primer sets (hA3A_PCR_F/hA3A_PCR_R) were used to amplify the fragment Human_APOBEC3A with template pUC57-Human_APOBEC3A (synthesized by Genscript). Then the fragment Human APOBEC3A was cloned into the SacI and SmaI linearized pCMV-BE3 (addgene, 73021) with plasmid recombination kit Clone Express® (Vazyme, C112-02) to generate the hA3A-BE3 expression vector pCMV-hAPOBEC3A-XTEN-D10A-SGGS-UGI-SGGS-NLS. hA3B-BE3, hA3C-BE3, hA3D-BE3, hA3F-BE3, hA3G-BE3, hA3H-BE3, hAID-BE3, hA1-BE3, mA3-BE3, mAID-BE3, mA1-BE3, cAICDA-BE3, expression vectors were constructed with the same strategy. The pmCDA1 expression vector pcDNA3.1_pCMV-nCas-PmCDA1-ugi pH1-gRNA (HPRT) was purchased from Addgene (79620).

Primer sets (SupF_PCR_F/SupF_PCR_R) were used to amplify the fragment SupF with template shuttle vector pSP189. Then the fragment SupF was cloned into pEASY-ZERO-BLUNT (TransGen Biotech, CB501) to generate the vector pEASY-SupF-ZERO-BLUNT.

Oligonucleotides SupF_sg1_FOR/SupF_sg1_REV and SupF_sg2_FOR/SupF_sg2_REV were annealed and ligated into BsaI linearized pGL3-U6-sgRNA-PGK-puromycin (addgene, 51133) to generate the sgRNA expression vectors psgSupF-1 and psgSupF-2 that target the SupF gene in pEASY-SupF-ZERO-BLUNT.

Two primer sets (hA3A_PCR_F/hA3A_Y130F_PCR_R) (hA3A_Y130F_PCR_F/hA3A_PCR_R) were used to amplify the Y130E-containing fragment hA3A-Y130F. Then the fragment was cloned into the ApaI and SmaI linearized hA3A-BE3 expression vector to generate the hA3A-BE3-Y130F expression vector pCMV-hAPOBEC3A_Y130E-XTEN-D10A-SGGS-UGI-SGGS-NLS. hA3A-BE3-D131Y, hA3A-BE3-Y132D, hA3A-BE3-C101S and hA3A-BE3-C106S expression vectors were constructed with the same strategy.

Primer sets (hA3A_PCR_F/hA3A_PCR_R) were used to amplify the fragment Human_APOBEC3A_Y130F with template hA3A-BE3-Y130F. Then the fragment Human_APOBEC3A_Y130F was cloned into the SacI and SmaI linearized pCMV-eBE-S3¹⁹ to generate the hA3A-eBE-Y130F expression vector pCMV-hAPOBEC3A_Y130F-XTEN-D10A-SGGS-UGI-SGGS-NLS-T2A-UGI-NLS-P2A-UGI-NLS-T2A-UGI-NLS. hA3A-eBE-Y132D expression vector was constructed by the similar way.

Oligonucleotides hEMX1_FOR/hEMX1_REV were annealed and ligated into BsaI linearized pGL3-U6-sgRNA-PGK-puromycin to generate sgEMX1 expression vector psgEMX1. Other sgRNA expression vectors were constructed with the same strategy.

### Antibodies

Antibodies were purchased from the following sources: against alpha-tubulin (T6199)—Sigma; against Cas9 (ab204448)—Abcam.

### Immunoblotting Analysis

Protein samples were incubated at 95° C. for 20 min, separated by SDS-PAGE in sample loading buffer and proteins were transferred to nitrocellulose membranes (Thermo Fisher Scientific). After blocking with TBST (25 mM Tris pH 8.0, 150 mM NaCl, and 0.1% Tween 20) containing 5% (w/v) nonfat dry milk for 2 h, the membrane was reacted overnight with indicated primary antibody. After extensive washing, the membranes were reacted with HRP-conjugated secondary antibodies for 1h. Reactive bands were developed in ECL (Thermo Fisher Scientific) and detected with Amersham Imager 600.

### Cell Culture and Transfection

HEK293T cells from ATCC were maintained in DMEM (10566, Gibco/Thermo Fisher Scientific)+10% FBS (16000-044, Gibco/Thermo Fisher Scientific) and regularly tested to exclude mycoplasma contamination.

The dCas9-Suntag-TetCD system was used to induce targeted demethylation of the genomic regions with natively high levels of methylation, e.g., FANCF, MAGEA1 and MSSK1 regions. The dCas9-DNMT3a-DNMT31 system was used to induce targeted methylation of the genomic regions with natively low levels of methylation, e.g., VEGFA and PDL1 regions. HEK293T cells were transfected by using LIPOFECTAMINE 2000 (Life, Invitrogen) with 3 μg pCAG-scFvGCN4sfGFPTET1CD (synthesized by Genscript) and 1 μg sgRNA expression vector or with 3 μg dCas9-DNMT3a-DNMT31 (synthesized by Genscript) and 1 μg sgRNA expression vector. Blasticidin (10 μg/ml, Sigma, 15205) and puromycin (1 μg/ml, Merck, 540411) were added 24 h after transfection. One week later, a portion of cells were collected to determine DNA methylation level and others were stored in liquid nitrogen for base editing. The sgRNAs used to induce genomic DNA methylation/demethylation are the ones used to induce base editing.

For base editing in genomic DNA, HEK293T cells were seeded in a 24-well plate at a density of 1.6×10⁵ per well and transfected with 200 μl serum-free Opti-MEM that contained 5.04 μl LIPOFECTAMINE LTX (Life, Invitrogen), 1.68 μl LIPOFECTAMINE plus (Life, Invitrogen), 1 μg BE3 expression vector (or hA3A-BE3, hA3A-BE3-Y130F, hA3A-BE3-D131Y, hA3A-BE3-Y132D, hA3A-BE3-C101S, hA3A-BE3-C106S, hA3A-eBE-Y130F, hA3A-eBE-Y132D expression vector) and 0.68 μg sgRNA expression vector. After 72 hr, the genomic DNA was extracted from the cells with QuickExtract™ DNA Extraction Solution (QE09050, Epicentre) or the cells were lysed in 2×SDS loading buffer for western blot.

For base editing in plasmid vector, 293T cells were seeded in a 6-well plate at a density of 3×10⁵ per well and transfected with 500 μl serum-free Opti-MEM that contained 4 μl LIPOFECTAMINE LTX (Life, Invitrogen), 2 μl LIPOFECTAMINE plus (Life, Invitrogen), 1 μg BE3 expression vector (or hA3A-BE3, hA3B-BE3, hA3C-BE3, hA3D-BE3, hA3F-BE3, hA3G-BE3, hA3H-BE3, hAID-BE3, hA1-BE3, mA3-BE3, mAID-BE3, mA1-BE3, cAICDA-BE3 or pmCDA1 expression vector) and 0.5 μg sgRNA expression vector. After 24 hr, these cells were transfected with 500 μl serum-free Opti-MEM that contained 4 μl LIPOFECTAMINE LTX, 2 μl LIPOFECTAMINE plus and 1.5 μg un-methylated (or methylated) pEASY-SupF-ZERO-BLUNT. After 48 hr, the plasmids were extracted from the cells with TIANprep Mini Plasmid Kit (DP103-A, TIANGEN) or the cells were lysed in 2×SDS loading buffer for western blot.

### Bisulfite Sequencing Analysis

Genomic DNA was isolated and treated with bisulfite according to the instruction of EZ DNA methylation-direct Kit (Zymo Research, D5021). The bisulfite-treated DNA was PCR-amplified with Taq™ Hot Start Version (Takara, R007B). The PCR products were ligated into T-Vector pMDTM19 (Takara, 3271). Eight clones were picked out and sequenced by Sanger sequencing (Genewiz). The primers used for bisulfite PCR were listed in Supplementary Table 2.

### Plasmid DNA Methylation

For in vitro methylation, 1 μl CpG methyltransferase (M.SssI, Life, EM0821) was used to methylate 2 μl plasmid DNA in a 20-μl reaction. After in vitro methylation, pEASY-SupF-ZERO-BLUNT was restricted with BstUI (NEB, R0518S) to determine the methylation level.

### Blue/White Colony Screening

The plasmids extracted from transfected cells were transformed into E. coli strain MBM7070 (lacZ^{uag_amber}), which were grown on LB plates containing 50 μg/ml kanamycin, 1 mM IPTG and 0.03% Bluo-gal (Life, Invitrogen) at 37° C. overnight and then at room temperature for another day (for maximal color development). The cumulative base editing frequency is calculated by dividing the number of white colonies with the number of total colonies.

### DNA Library Preparation and Sequencing

Target genomic sites were PCR amplified by high-fidelity DNA polymerase PrimeSTAR HS (Clonetech) with primers flanking each examined sgRNA target site. The PCR primers used to amplify target genomic sequences were listed in Supplementary Table 2. Indexed DNA libraries were prepared by using the TruSeq ChIP Sample Preparation Kit (Illumina) with some minor modifications. Briefly, the PCR products were fragmented by Covaris 5220 and then amplified by using the TruSeq ChIP Sample Preparation Kit (Illumina). After being quantitated with Qubit High-Sensitivity DNA kit (Life, Invitrogen), PCR products with different tags were pooled together for deep sequencing by using the Illumina NextSeq 500 (2×150) or Hiseq X Ten (2×150) at CAS-MPG Partner Institute for Computational Biology Omics Core, Shanghai, China. Raw read qualities were evaluated by FastQC. For paired-end sequencing, only R1 reads were used. Adaptor sequences and read sequences on both ends with Phred quality score lower than 28 were trimmed. Trimmed reads were then mapped with the BWA-MEM algorithm (BWA v0.7.9a) to target sequences. After being piled up with samtools (v0.1.18), indels and base substitutions were further calculated.

### Indel Frequency Calculation

Indels were estimated in the aligned regions spanning from upstream eight nucleotides of the target site to downstream 19 nucleotides of PAM sites (50 bp). Indel frequencies were subsequently calculated by dividing reads containing at least one inserted and/or deleted nucleotide by all the mapped reads at the same region.

### Base Substitution Calculation

Base substitutions were selected at each position of the examined sgRNA target sites that mapped with at least 1,000 independent reads, and obvious base substitutions were only observed at the targeted base editing sites. Base substitution frequencies were calculated by dividing base substitution reads by total reads.

### Calculation of BE-Targetable Genetic Variants

The single nucleotide variants (SNVs) from NCBI ClinVar database were overlapped with the pathogenic human allele sequence from NCBI dbSNP database to calculate the pathogenic T-to-C and A-to-G mutations. In 3,089 pathogenic T-to-C or A-to-G mutations, 2,499 are potentially editable by SpCas9-BE3, SaCas9-BE3, dLbCpf1-BE or xCas9-BE3 with nearby PAM sequences. These 2,499 BE-targetable SNVs are further sub-classified according to their 3′ adjacent base preferences, i.e., CpA, CpC, CpG and CpT (FIG. 5a).

### Statistical Analysis

P values were calculated from one-tailed Student's t test in this study.

### Data Availability

The deep-sequencing data from this study are deposited in the NCBI Gene Expression Omnibus (accession no. GSE114999) and the National Omics Data Encyclopedia (accession no. OEP000030).

### Results

This example first examined the base editing efficiency of a commonly used BE, the rat APOBEC1 (rA1)-based BE3, in human cells having either increased or decreased levels of methylation. When DNA methylation was promoted by DNMT3 in regions with native low methylation levels, editing frequencies by BE3 decreased. In addition, when DNA methylation was reduced by TET1 in regions with native high methylation levels, BE3-induced editing frequencies increased accordingly. These results suggest that the canonical rA1-based BE3 is less efficient in editing cytosines embedded in highly methylated genomic regions. Notably, C-to-T editing was suppressed by DNA methylation at both CpG and flanking non-CpG sites (median decrement ˜28%, P=2×10⁻⁸ for CpG sites and ˜51%, P=7×10⁻¹⁰ for flanking non-CpG sites). APOBECs deaminate cytidines on single-stranded DNA in a processive manner. CpG methylation may affect the sliding of APOBEC and therefore impairs its binding on the flanking non-CpG sites for deamination.

To screen for efficient base editing in high-methylation background, a series of BEs was obtained by fusing Cas9 nickase with fifteen different APOBEC/AID deaminases (FIG. 5b). This example tested these BEs then in an E. coif-derived vector system (FIG. 5b), which has been previously used to probe mutations. In unmethylated vectors, these BEs showed varied levels of base editing. The BEs containing human APOBEC3A (hA3A-BE3, mean editing frequency ˜39%), human APOBEC3B (hA3B-BE3, mean editing frequency ˜33%) or human AID (hAID-BE3, mean editing frequency ˜28%) mediated base editing at levels that are comparable to BE3 (mean editing frequency ˜31%) (FIG. 5c). Whereas in methylated vectors, only hA3A-BE3 induced efficient base editing (mean editing frequency ˜35%), compared to relatively low editing efficiencies induced by BE3 (mean editing frequency ˜12%) or other examined BEs (mean editing frequencies ˜1%-20%) (FIG. 5c). Of note, protein products of hA3A-BE3, BE3 and other examined BEs are comparable (FIG. 5d).

Similar to the observation in E. coif-derived vectors, hA3A-BE3 exhibited significantly higher base editing frequencies than rA1-based BE3 in all tested genomic regions, either those with a native high-methylation background (median ˜1.7-fold, P=2×10⁻¹⁰, FIG. 5e,f) or those with an induced high-methylation condition (median ˜1.8-fold, P=5×10⁻⁴). Thus, using hA3A as the deaminase module in BE could generally achieve high base editing efficiency in genomic regions with high methylation levels.

The base editing on cytosines in a GpC context was observed to be generally inefficient by rA1-based BEs. While, this example found that hA3A-BE3 could induce efficient base editing on most of cytosines at GpC sites in both endogenously and induced high-methylation backgrounds (FIG. 5e). This example further compared their editing efficiencies under both endogenously and induced low-methylation backgrounds and observed a similar superiority of hA3A-BE3 over BE3 on editing cytosines in the GpC context (FIG. 5g,h). Statistical analysis confirmed that the base editing efficiency induced by hA3A-BE3 was significantly higher than that induced by BE3 on cytosines in the GpC context in either high- (median ˜2.3-fold, P=1×10⁻⁵) or low- (median ˜1.8-fold, P=6×10⁻⁹) methylation conditions. Notably, hA3A-BE3-mediated base editing was as efficient as BE3 at cytosines in non-GpC contexts in all tested low-methylation regions (median ˜1.1-fold, P=0.045). This example also found that hA3A-BE3 yielded less non-C-to-T conversion than BE3 in both high- (median ˜97% by hA3A-BE3 comparing to ˜94% by BE3, P=3×10⁻⁴) and low-methylation regions (median ˜92% by hA3A-BE3 comparing to ˜90% by BE3, P=4×10⁻⁶). Both BE3 and hA3A-BE3 induced less non-C-to-T conversion at CpG sites with high methylation status than at CpG sites with low methylation status (median ˜95% vs ˜90%, P=3×10⁻⁵ for BE3 and median ˜95% vs ˜92%, P=5×10⁻⁴ for hA3A-BE3). This example also found that hA3A-BE3 induced higher indel frequencies than BE3 (median ˜2 in both high- and low-methylation regions). Such an increase may be caused by the high deaminase activity of hA3A, which can trigger downstream DNA repair pathways to generate DNA double strand breaks.

The results suggest that hA3A-BE3 can efficiently induce base editing in a broader scope (FIG. 5). However, the editing window of hA3A-BE3 is wider (˜12 nt, position 2-13 in the sgRNA target site) than that of BE3 (˜5 nt, position 4-8). As the wide editing window of hA3A-BE3 may result from the high deaminase activity of hA3A, mutations in hA3A that can reduce deaminase activity might correspondingly narrow the editing window of hA3A-BE3. Designated mutations (Y130F, D131Y or Y132D) successfully narrowed the editing window with little effect on the base editing efficiency, whereas mutations in the zinc-coordination motif almost completely eliminated the deaminase activity (C101S and C106S).

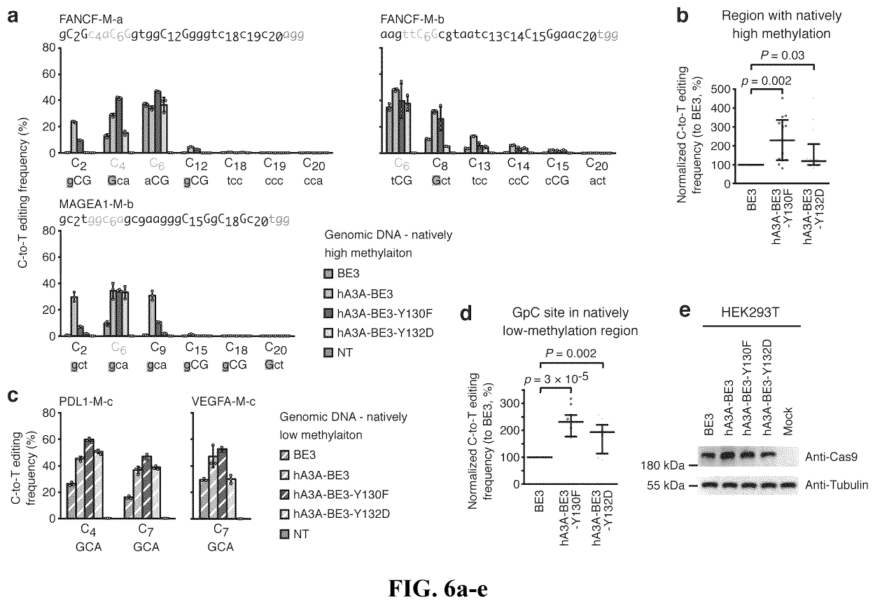

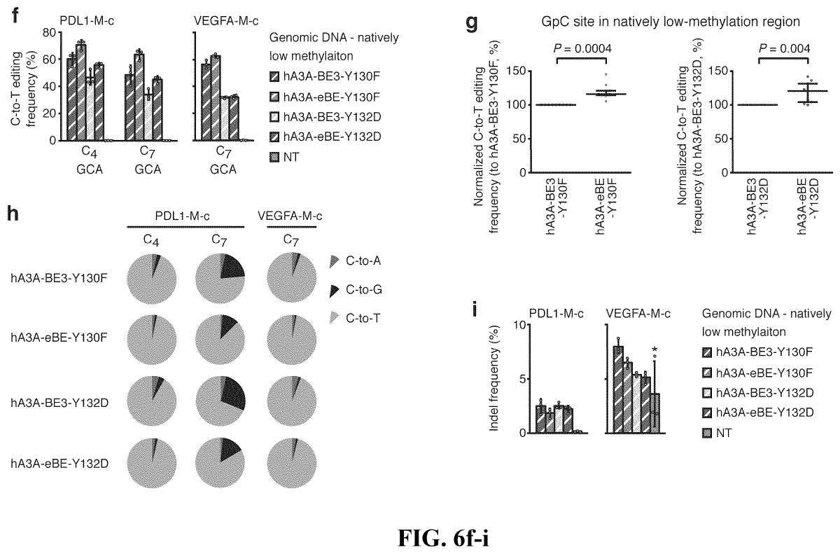

This example then focused on two engineered hA3A-BE3s (hA3A-BE3-Y130F and hA3A-BE3-Y132D), which have similar editing windows (position 3-8 for hA3A-BE3-Y130F and position 3-7 for hA3A-BE3-Y132D) as BE3 (position 4-8). In highly-methylated regions, hA3A-BE3-Y130F and hA3A-BE3-Y132D induced higher editing efficiencies than BE3 at all editable sites in overlapping editing windows (position 4-7) (FIG. 6a, cytosines in pink and FIG. 6b, median ˜2.3 fold, P=0.002 for hA3A-BE3-Y130F and median ˜1.2 fold, P=0.03 for hA3A-BE3-Y132D). For cytosines outside of overlapping editing windows, hA3A-BE3-Y132D induced C-to-T editing frequencies similar to BE3 while hA3A-BE3-Y130F induced higher editing frequencies (FIG. 6a, cytosines in black). Similar to the original hA3A-BE3, both engineered hA3A-BE3-Y130F and hA3A-BE3-Y132D edited cytosines in GpC contexts more efficiently than BE3 in overlapping editing windows (FIG. 6c,d, median ˜2.3 fold, P=3×10⁻⁵ for hA3A-BE3-Y130F and median ˜1.9 fold, P=0.002 for hA3A-BE3-Y132D). Protein expression levels of hA3A-BE3-Y130F and hA3A-BE3-Y132D were very similar to that of BE3 (FIG. 6e), though the two engineered hA3A-BEs induced higher C-to-T editing efficiencies (FIG. 6b,d). In terms of product purity, we found that hA3A-BE3-Y130F yielded less non-C-to-T conversion (median ˜96.3% by hA3A-BE3-Y130F comparing to ˜95.6% by BE3, P=0.03 in high-methylation regions, median ˜92% by hA3A-BE3-Y130F comparing to ˜90% by BE3, P=0.002 in low-methylation regions) but more indels (median ˜2.1 fold, P=0.0002 in high-methylation regions, median ˜1.3 fold in low-methylation regions, P=0.12) than BE3. The product purity induced by hA3A-BE3-Y132D was higher than BE3 in native low-methylation regions (median ˜93% by hA3A-BE3-Y132D comparing to ˜90% by BE3, P=0.001), but lower in native high-methylation regions (median ˜94.9% by hA3A-BE3-Y132D comparing to ˜95.6% by BE3, P=0.03). Nevertheless, indel frequencies induced by hA3A-BE3-Y132D were comparable to those induced by BE3 at all tested sites (median ˜1.2 fold in both high- and low-methylation regions).

To further enhance C-to-T base editing system, three copies of the 2A-uracil DNA glycosylase inhibitor (UGI) sequence were fused to the C-terminus of hA3A-BE3-Y130F and hA3A-BE3-Y132D to develop hA3A-eBE-Y130F and hA3A-eBE-Y132D. In low-methylation regions, hA3A-eBE-Y130F and hA3A-eBE-Y132D induced significantly higher editing efficiencies (FIG. 6f,g, median ˜1.2 fold, P=0.0004 for hA3A-eBE-Y130F and median ˜1.2 fold, P=0.004 for hA3A-eBE-Y132D), higher product purity (FIG. 6h, median ˜96% by hA3A-eBE-Y130F comparing to ˜94% by hA3A-BE3-Y130F, P=0.006 and median ˜96% by hA3A-eBE-Y132D comparing to ˜92% by hA3A-BE3-Y132D, P=0.004) and lower indel frequencies (FIG. 6i, median decrement ˜21%, P=4×10⁻⁵ for hA3A-eBE-Y130F and median decrement ˜9%, P=0.03 for hA3A-eBE-Y132D) than hA3A-BE3-Y130F and hA3A-BE3-Y132D, respectively. In high-methylation regions, hA3A-eBE-Y130F and hA3A-eBE-Y132D induced significantly higher product purity (median ˜97% by hA3A-eBE-Y130F comparing to ˜95% by hA3A-BE3-Y130F, P=0.003 and median ˜97% by hA3A-eBE-Y132D comparing to ˜95% by hA3A-BE3-Y132D, P=0.003) and lower indel frequencies (median decrement ˜23%, P=2×10⁻⁷ for hA3A-eBE-Y130F and median decrement ˜21%, P=4×10⁻⁵ for hA3A-eBE-Y132D) than hA3A-BE3-Y130F and hA3A-BE3-Y132D, respectively, though editing efficiencies remained the same (median ˜1 fold for hA3A-eBE-Y130F and hA3A-eBE-Y132D). Together, these results indicated that hA3A-BE3-Y130F, hA3A-BE3-Y132D, hA3A-eBE-Y130F and hA3A-eBE-Y132D can mediate highly efficient base editing in narrowed editing windows compared to the original hA3A-BE3 in all examined contexts.

Here, this example demonstrates that hA3A-BE3 and its engineered forms, can comprehensively induce efficient base editing in all examined contexts, including both methylated DNA regions and GpC dinucleotides. It is contemplated that hA3A can also be conjugated with other Cas proteins to further expand the scope of base editing.

### Example 3. Gene Editing of Human DYRK1A with dCas12a-hA3A Base Editors

This example tested base editors that combined a Cas12a (Cpf1) and various mutant human A3A proteins.

### Methods

Construction of dCas12a-hA3A-BE Expression Vector

pUC57-hA3A (synthesized by Genscript Biotechnology Co., Ltd.) was used as a template, using suitable primers. PCR was carried out to obtain the coding sequence of hA3A, and a fragment homologous to the linearized vector at both ends was subjected to gel electrophoresis purification. After purification by gel electrophoresis, the fragment was recombined into the linearized dCas12a-BE vector produced by SacI and SmaI by plasmid recombinant kit Clone Express® to obtain expression vector dCas12a-hA3A-BE.

Construction of dCas12a-hA3A-BE-W98Y Expression Vector

Using dCas12a-hA3A-BE as a template, two PCR products with a W98Y mutation and a homology arm, and a homologous segment with a linearized vector. After purification by gel electrophoresis, the two fragments were simultaneously recombined into the linearized dCas12a-hA3A-BE vector generated by ApaI and SmaI using plasmid recombinant kit Clone Express® to obtain expression vector dCas12a-hA3A-BE-W98Y.

Likewise, expression vectors dCas12a-hA3A-BE-W104A, dCas12a-hA3A-BE-P134Y, dCas12a-hA3A-BE-W98Y-W104A, dCas12a-hA3A-BE-W98Y-P134Y, dCas12a-hA3A-BE-W104A-P134Y, dCas12a-hA3A-BE-W98Y-W104A-Y130F, dCas12a-hA3A-BE-W98Y-W104A-Y132D, dCas12a-hA3A-BE-W104A-Y130E-P134Y, and dCas12a-hA3A-BE-W104A-Y132D-P134Y. Relevant sequences are shown in Tables 1 and 2.

Construction of gRNA Expression Plasmid

The nucleotide sequence was annealed to primers and the annealed product was ligated into the gRNA expression vector pLb-Cas12a-pGL3-U6-sgRNA digested with restriction endonuclease BsaI using T4 DNA ligase. gRNA expression plasmid sgDYRK1A targeting human DYRK1A site was obtained.

### Eukaryotic Cell Transfection

The sgDYRK1A and each of dCas12a-hA3A-BE, dCas12a-hA3A-BE-W98Y, dCas12a-hA3A-BE-W104A, dCas12a-hA3A-BE-P134Y, dCas12a-hA3A-BE-W98Y-W104A, dCas12a-hA3A-BE-W98Y-P134Y, dCas12a-hA3A-BE-W104A-P134Y, dCas12a-hA3A-BE-W98Y-W104A-Y130F, dCas12a-hA3A-BE-W98Y-W104A-Y132D, dCas12a-hA3A-BE-W104A-Y130E-P134Y, dCas12a-hA3A-BE-W104A-Y132D-P134Y were mixed into 200 μl Opti-MEM at a ratio of 0.68 ug:1 μg, added with 1.68 μl of LIPOFECTAMINE plus, and 5.04 μl of LIPOFECTAMINE LTX was added, and allowed to stand at room temperature for 5 minutes. 500 μl DMEM (+10% FBS) medium was add for 24-well plates and transfected HEK293T cells 160,000. After 12 h, replaced with fresh medium containing 1% double antibody (cyanin). The cells were harvested after 60 hours of incubation.

### EditR Analysis of Sanger Sequencing Results

DNA sanger sequencing results were analyzed using EditR software (moriaritylab.shinyapps.io/editr_v10/). EditR is a web version of the sanger sequencing result analysis software developed in 2018 (Kluesner M G, Nedveck D A, Lahr W S, et al. EditR: A Method to Quantify Base Editing from Sanger Sequencing [J]. The CRISPR Journal, 2018, 1 (3): 239-250.). EditR is a simple, accurate and efficient analytical tool for processing the sequencing results of DNA samples based on the sgRNA sequence by using the sanger sequencing signal, and finally outputting the base editing efficiency at the sgRNA target site.

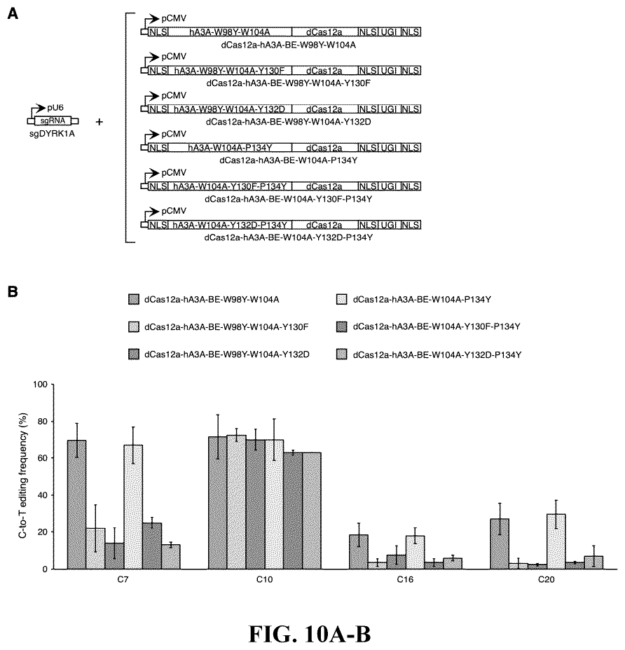

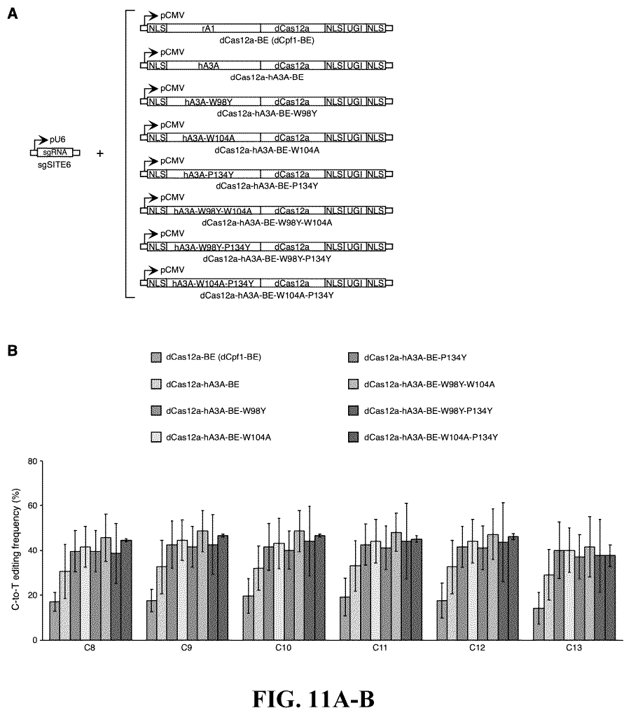

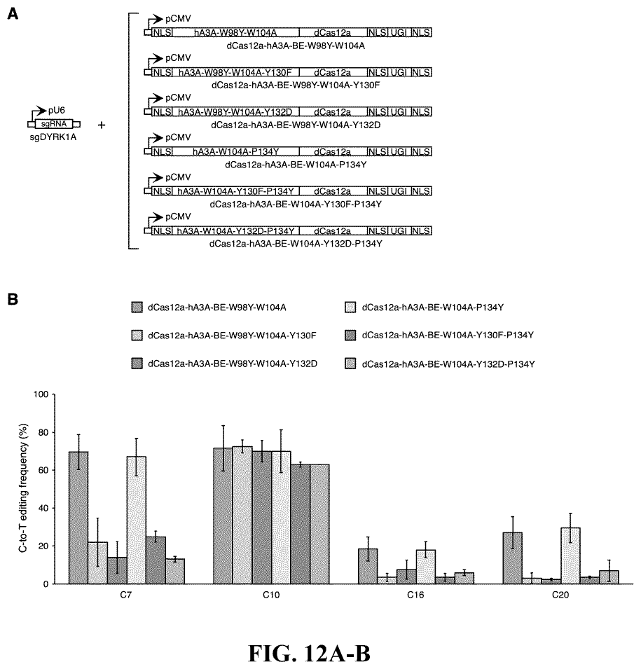

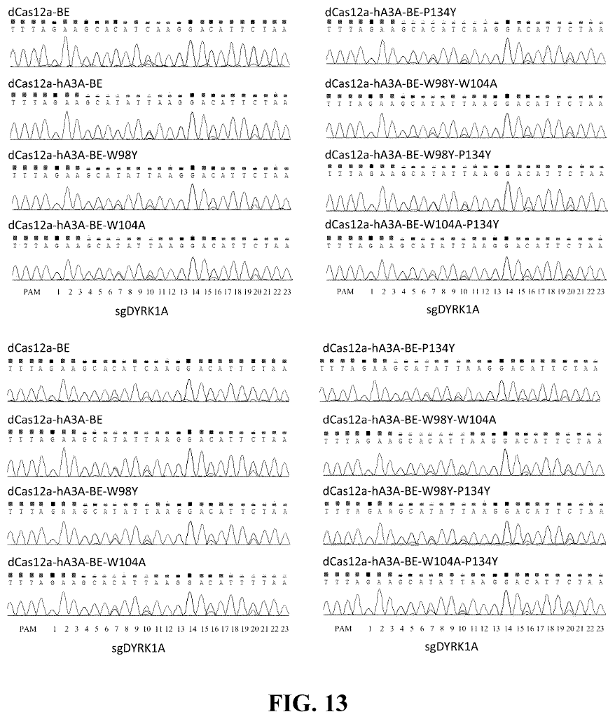

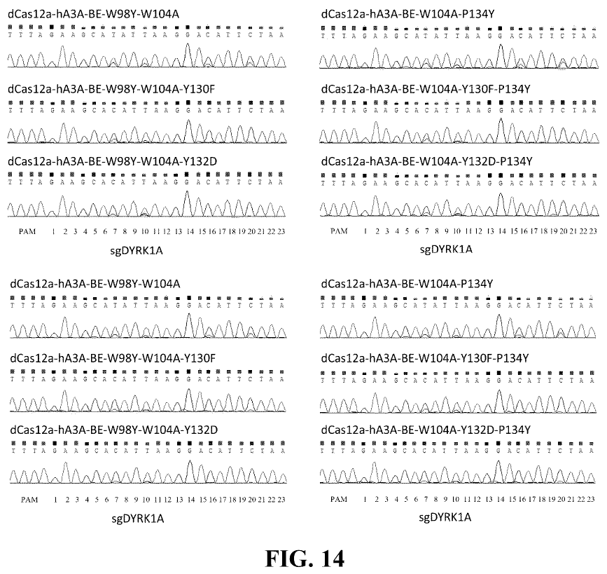

The sequencing results are shown FIGS. 11 and 12. The EditR analysis results are presented in FIGS. 7 and 8. When fused to the conventional cytosine deaminase, A1 (APOBEC1), Cas12a (cpf1) exhibited poor efficiency (see, e.g., FIG. 7B, the first column in each group). The combination with the hA3A wild-type protein greatly increased the editing efficiency (see, e.g., the second column). Interestingly, the A3A mutation W98Y, W104A, P134Y or the combination of each two further increased the editing efficiency (FIG. 7). Also, the editing window such a Cas12a-A3A can be narrowed to achieve more precise editing when the mutation Y130F or Y132D is further included in A3A (FIG. 8).

### Example 4. Gene Editing of Human SITE6 with dCas12a-hA3A Base Editors

This example tested various indicated base editors with the human gene SITE6.

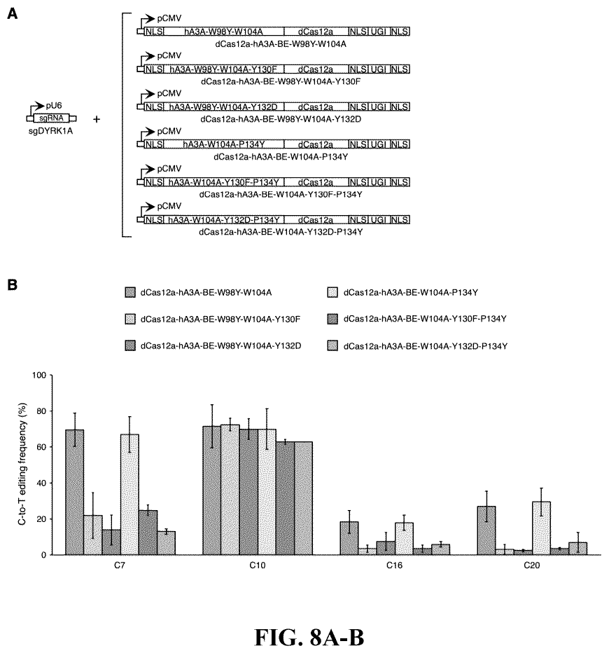

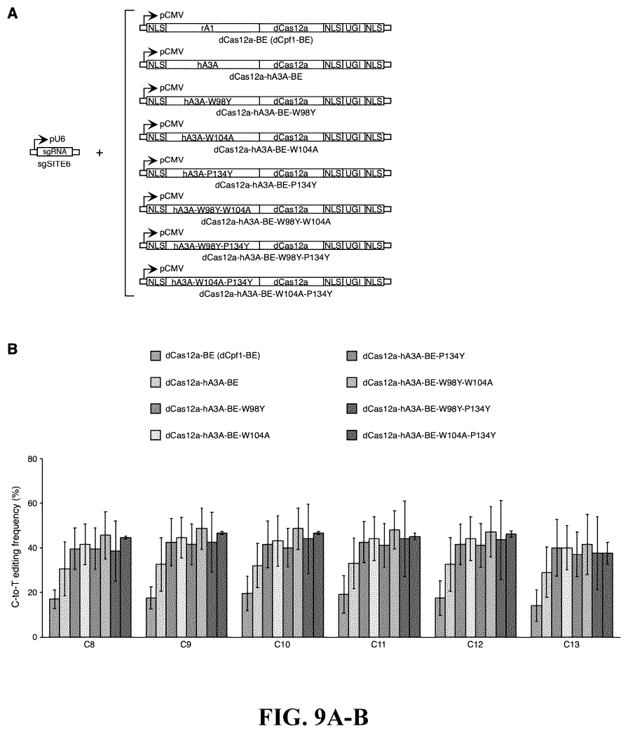

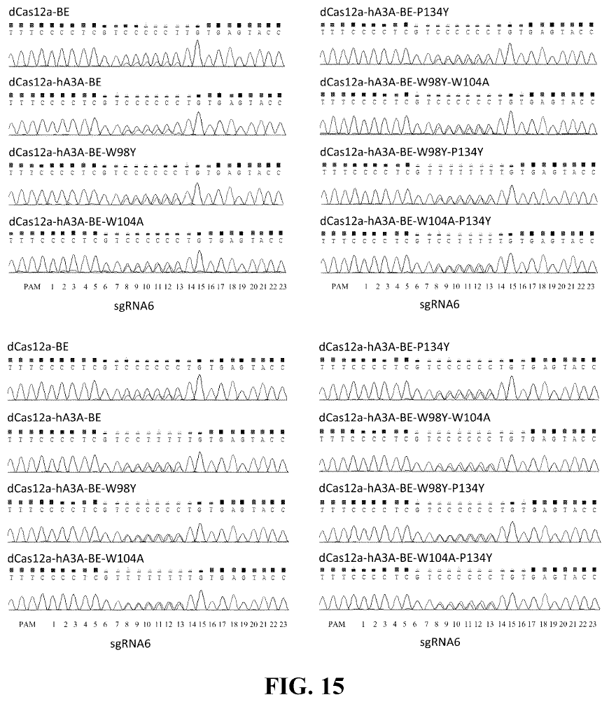

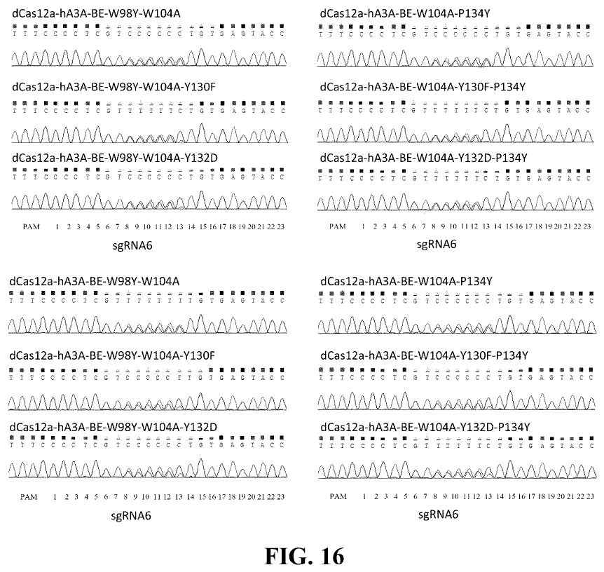

The experimental procedure is similar to Example 3. The sequencing results are shown in detail in FIGS. 15 and 16 (two replicates of experimental data). The EditR analysis results are shown in FIGS. 9 and 10. Like in Example 3, the Cas12a-A3A editor had greater editing efficiency than the Cas12a-A1 and the A3A mutation W98Y, W104A, P134Y or the combination of each two further increased the editing efficiency (FIG. 9). Also, the editing window such a Cas12a-A3A can be narrowed to achieve more precise editing when the mutation Y130F or Y132D is further included in A3A (FIG. 10).

### Example 5. Gene Editing of Human RUNX1 with dCas12a-hA3A Base Editors

This example tested various indicated base editors with the human gene RUNX1.

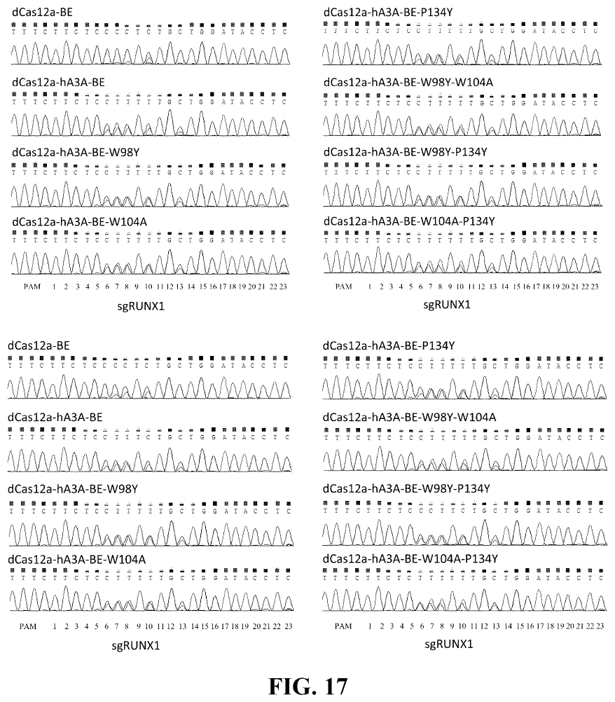

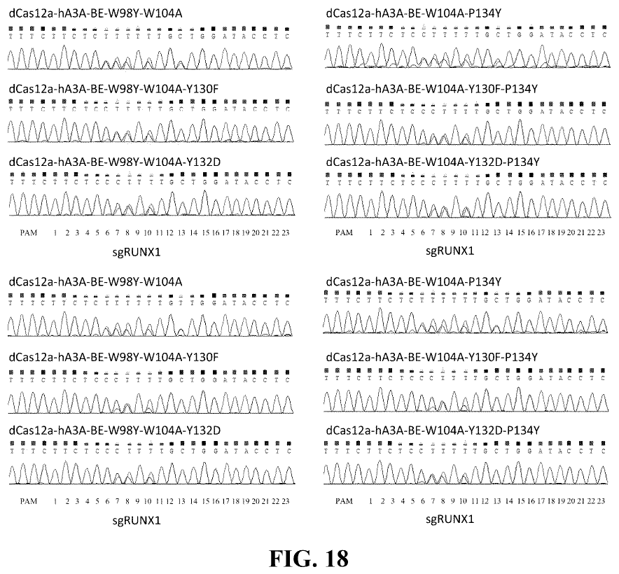

The experimental procedure is similar to Example 3. The sequencing results are shown in detail in FIGS. 17 and 18 (two replicates of experimental data). The EditR analysis results are shown in FIGS. 11 and 12. Like in Example 3, the Cas12a-A3A editor had greater editing efficiency than the Cas12a-rA1, and the A3A mutation W98Y, W104A, P134Y or the combination of each two further increased the editing efficiency (FIG. 11). Also, the editing window such a Cas12a-A3A can be narrowed to achieve more precise editing when the mutation Y130F or Y132D is further included in A3A (FIG. 12).

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

What is claimed is:

1. A method for deaminating a cytosine (C) in a GpC context in a target polynucleotide, comprising contacting the target polynucleotide with a fusion protein and a guide RNA having at least partial sequence complementarity to the target polynucleotide, wherein the fusion protein comprises a first fragment comprising an apolipoprotein B mRNA editing enzyme catalytic subunit 3A (APOBEC3A) and a second fragment comprising a clustered regularly interspaced short palindromic repeats (CRISPR)-associated (Cas) protein.

2. The method of claim 1, wherein the C is methylated.

3. The method of claim 2, wherein the target polynucleotide is in a cell.

4. The method of claim 3, wherein the contacting is in vivo.

5. The method of claim 1, wherein the APOBEC3A is a mutant of human APOBEC3A having a mutation selected from the group consisting of D131Y, Y132D, W104A, P134Y and combinations thereof, according to residue numbering in SEQ ID NO:1, wherein the amino acid sequence of the fusion protein has at least 85% sequence identity to SEQ ID NO:1, and wherein the mutant retains cytidine deaminase activity.

6. The method of claim 5, wherein the mutant human APOBEC3A has mutations selected from the group consisting of Y130F+D131E+Y132D, Y130F+D131Y+Y132D, W98Y+W104A, W98Y+P134Y, W104A+P134Y, W104A+Y130F, W104A+Y132D, W98Y+W104A+Y130F, W98Y+W104A+Y132D, W104A+Y130F+P134Y, and W104A+Y132D+P134Y, according to residue numbering in SEQ ID NO:1.

7. The method of claim 1, wherein the human APOBEC3A is human APOBEC3A isoform a or isoform b.

8. The method of claim 1, wherein the APOBEC3A comprises an amino acid sequence selected from the group consisting of SEQ ID NO:3-5, 22-23, 25-34.

9. The method of claim 1, wherein the Cas protein is selected from the group consisting of Streptococcus pyogenes CRISPR-associated protein (SpCas9), Francisella novicida Cas9 (FnCas9), Streptococcus thermophilus CRISPR-1 Cas9 (St1Cas9), Streptococcus thermophilus CRISPR-3 Cas9 (St3Cas9), NmCas9, SaCas9, AsCpf1, LbCpf1, FnCpf1, D1135V/R1335Q/T1337R (VQR) SpCas9, D1135E/R1335Q/T1337R (EQR) SpCas9, D1135V/G1218R/R1335E/T1337R (VRER) SpCas9, E1369R/E1449H/R1556A (RHA) FnCas9, E782K/N968K/R1015H (KKH) Staphylococcus aureus Cas9 (SaCas9), Neisseria meningitidis Cas9 (NmeCas9), Streptococcus thermophilus Cas9 (StCas9), Campylobacter jejuni (CjCas9), Acidaminococcus sp. Cpf1 (AsCpf1), Franscisella novicida Cpf1 (FnCpf1), Smithella sp. Cpf1 (SsCpf1), Porphyromonas crevioricanis Cpf1 (PcCpf1), Butyrivibrio proteoclasticus Cpf1 (BpCpf1), Candidatus Methanoplasma termitum (CmtCpf1), Leptospira inadai Cpf1 (LiCpf1), Porphyromonas macacae Cpf1 (PmCpf1), Parcubacteria bacterium 3310 Cpf1 (Pb3310Cpf1), Parcubacteria bacterium 4417 Cpf1 (Pb4417Cpf1), Butyrivibrio sp. NC3005 Cpf1 (BsCpf1), Eubacterium eligens Cpf1 (EeCpf1), Bacillus hisashii Cas12b (BhCas12b), Alicyclobacillus kakegawensis Cas12b (AkCas12b), Elusimicrobia bacterium Cas12b (EbCas12b), Laceyella sediminis Cas12b (LsCas12b), Ruminococcus flavefaciens Cas13d (RfCas13d), Leptotrichia wadei Cas13a (LwaCas13a), Prevotella sp. Cas13b (PspCas13b), Porphyromonas gulae Cas13b (PguCas13b), Porphyromonas gulae Cas13b (RanCas13b), CasX, and CasY.

10. The method of claim 1, wherein the Cas protein is a mutant of protein selected from the group consisting of SpCas9, FnCas9, St1Cas9, St3Cas9, NmCas9, SaCas9, AsCpf1, LbCpf1, FnCpf1, VQR SpCas9, EQR SpCas9, VRER SpCas9, RHA FnCas9, KKH SaCas9, NmeCas9, StCas9, CjCas9, AsCpf1, FnCpf1, SsCpf1, PcCpf1, BpCpf1, CmtCpf1, LiCpf1, PmCpf1, Pb3310Cpf1, Pb4417Cpf1, BsCpf1, EeCpf1, BhCas12b, AkCas12b, EbCas12b, LsCas12b, RfCas13d, LwaCas13a, PspCas13b, PguCas13b, RanCas13b, CasX, and CasY, wherein the mutant retains the DNA-binding capability but does not introduce double strand DNA breaks.

11. The method of claim 10, wherein the mutant Cas protein is capable of introducing a nick to one of the strands of a double stranded DNA bound by the mutant.

12. The method of claim 10, wherein the mutant Cas protein comprises an amino acid sequence selected from the group consisting of SEQ ID NO:11, and 37-39.

13. The method of claim 1, wherein the first fragment is at the N-terminal side of the second fragment.

14. The method of claim 1, further comprising contacting the target polynucleotide with a uracil glycosylase inhibitor (UGI) not fused to a Cas protein.
